(19) United States
(12) Patent Application Publication
(10) Pub. No.: US 2003/0004310 A1
(43) Pub. Date: Jan. 2, 2003

(54) Novel amino acid sequences for human fetal brain-like polypeptides

(75) Inventors: Richard A. Shimkets, West Haven, CT (US); Elma Fernandes, Branford, CT (US)

Correspondence Address: MINTZ, LEVIN, COHN, FERRIS,, GLOVSKY AND POPEO, P.C., One Financial Center, Boston, MA, 02111, US

(73) Assignee: CuraGen Corporation, New Haven, CT

(21) Appl. No.: 10/004,551

(22) Filed: Dec. 5, 2001

Related U.S. Application Data
(63) Continuation of application No. 09/635,949, filed on Aug. 10, 2000.
(60) Provisional application No. 60/148,433, filed on Aug. 11, 1999.

(51) Int. Cl. A61K 38/17; C07H 21/04; C12P 21/02; C12N 5/06; C07K 14/435

(52) U.S. Cl. 530/350; 435/69.1; 435/320.1; 514/12; 435/325; 536/23.2

(57) ABSTRACT

This application is drawn to novel amino acid sequences for mammalian polypeptides that have sequence similarity to fetal brain tissue protein. The polypeptides are novel secreted proteins 649 amino acids in length.

### RELATED APPLICATIONS

This application claims priority to U.S. Ser. No. 09/635,949 filed Aug. 10, 2000, pending, which claims the benefit of U.S. Ser. No. 60/148,433 filed Aug. 11, 1999, abandoned.

### FIELD OF THE INVENTION

The invention relates to polynucleotides and polypeptides.

### BACKGROUND OF THE INVENTION

Eukaryotic cells are subdivided by membranes into multiple functionally distinct compartments called organelles. Each organelle includes proteins essential for its proper function. These proteins can include sequence motifs often referred to as sorting signals. The sorting signals can aid in targeting the proteins to their appropriate cellular organelle. In addition, sorting signals can direct some proteins to be exported, or secreted, from the cell.

One type of sorting signal is a signal sequence, which is also referred to as a signal peptide or leader sequence. The signal sequence is present as an amino-terminal extension on a newly synthesized polypeptide chain. A signal sequence can target proteins to an intracellular organelle called the endoplasmic reticulum (ER).

The signal sequence takes part in an array of protein-protein and protein-lipid interactions that result in translocation of a polypeptide containing the signal sequence through a channel in the ER. After translocation, a membrane-bound enzyme, named a signal peptidase, liberates the mature protein from the signal sequence.

The ER functions to separate membrane-bound proteins and secreted proteins from proteins that remain in the cytoplasm. Once targeted to the ER, both secreted and membrane-bound proteins can be further distributed to another cellular organelle called the Golgi apparatus. The Golgi directs the proteins to other cellular organelles such as vesicles, lysosomes, the plasma membrane, mitochondria and microbodies.

Only a limited number of genes encoding human membrane-bound and secreted proteins have been identified. Examples of known secreted proteins include human insulin, interferon, interleukins, transforming growth factor-beta, human growth hormone, erythropoietin, and lymphokines.

### SUMMARY OF THE INVENTION

The invention is based, in part, upon the discovery of novel nucleic acids and secreted polypeptides encoded thereby. The nucleic acids and polypeptides are collectively referred to herein as “PROX” nucleic acids and polypetpides.

Accordingly, in one aspect, the invention includes an isolated nucleic acid that encodes a PROX polypeptide, or a fragment, homolog, analog or derivative thereof. For example, the nucleic acid can encode a polypeptide at least 85% identical to a polypeptide comprising the amino acid sequences of SEQ ID NO:2, 4, 6, 8, 10, 12, 14, 16, 18, 20, 22, 24, 26, 28, 30, 32, and/or 34. The nucleic acid can be, e.g., a genomic DNA fragment, cDNA molecule. In some embodiments, the nucleic acid includes the sequence the invention provides an isolated nucleic acid molecule that includes the nucleic acid sequence of any of SEQ ID NO:1, 3, 5, 7, 9, 11, 13, 15, 17, 19, 21, 23, 25, 27, 29, 31, and/or 33.

Also included within the scope of the invention is a vector containing one or more of the nucleic acids described herein, and a cell containing the vectors or nucleic acids described herein.

The invention is also directed to host cells transformed with a vector comprising any of the nucleic acid molecules described above.

In another aspect, the invention includes a pharmaceutical composition that includes a PROX nucleic acid and a pharmaceutically acceptable carrier or diluent.

In a further aspect, the invention includes a substantially purified PROX polypeptide, e.g., any of the PROX polypeptides encoded by a PROX nucleic acid, and fragments, homologs, analogs, and derivatives thereof. The invention also includes a pharmaceutical composition that includes a PROX polypeptide and a pharmaceutically acceptable carrier or diluent.

In a still a further aspect, the invention provides an antibody that binds specifically to a PROX polypeptide. The antibody can be, e.g., a monoclonal or polyclonal antibody, and fragments, homologs, analogs, and derivatives thereof. The invention also includes a pharmaceutical composition including PROX antibody and a pharmaceutically acceptable carrier or diluent. The invention is also directed to isolated antibodies that bind to an epitope on a polypeptide encoded by any of the nucleic acid molecules described above.

The invention also includes kits comprising any of the pharmaceutical compositions described above.

The invention further provides a method for producing a PROX polypeptide by providing a cell containing a PROX nucleic acid, e.g., a vector that includes a PROX nucleic acid, and culturing the cell under conditions sufficient to express the PROX polypeptide encoded by the nucleic acid. The expressed PROX polypeptide is then recovered from the cell. Preferably, the cell produces little or no endogenous PROX polypeptide. The cell can be, e.g., a prokaryotic cell or eukaryotic cell.

The invention is also directed to methods of identifying a PROX polypeptide or nucleic acids in a sample by contacting the sample with a compound that specifically binds to the polypeptide or nucleic acid, and detecting complex formation, if present.

The invention further provides methods of identifying a compound that modulates the activity of a PROX polypeptide by contacting PROX polypeptide with a compound and determining whether the PROX polypeptide activity is modified.

The invention is also directed to compounds that modulate PROX polypeptide activity identified by contacting a PROX polypeptide with the compound and determining whether the compound modifies activity of the PROX polypeptide, binds to the PROX polypeptide, or binds to a nucleic acid molecule encoding a PROX polypeptide.

In a another aspect, the invention provides a method of determining the presence of or predisposition of a PROX-associated disorder in a subject. The method includes providing a sample from the subject and measuring the amount of PROX polypeptide in the subject sample. The amount of PROX polypeptide in the subject sample is then compared to the amount of PROX polypeptide in a control sample. An alteration in the amount of PROX polypeptide in the subject protein sample relative to the amount of PROX polypeptide in the control protein sample indicates the subject has a tissue proliferation-associated condition. A control sample is preferably taken from a matched individual, i e., an individual of similar age, sex, or other general condition but who is not suspected of having a tissue proliferation-associated condition. Alternatively, the control sample may be taken from the subject at a time when the subject is not suspected of having a tissue proliferation-associated disorder. In some embodiments, the PROX is detected using a PROX antibody.

In a further aspect, the invention provides a method of determining the presence of or predisposition of a PROX-associated disorder in a subject. The method includes providing a nucleic acid sample (e.g., RNA or DNA, or both) from the subject and measuring the amount of the PROX nucleic acid in the subject nucleic acid sample. The amount of PROX nucleic acid sample in the subject nucleic acid is then compared to the amount of a PROX nucleic acid in a control sample. An alteration in the amount of PROX nucleic acid in the sample relative to the amount of PROX in the control sample indicates the subject has a tissue proliferation-associated disorder.

In a still further aspect, the invention provides method of treating or preventing or delaying a PROX-associated disorder. The method includes administering to a subject in which such treatment or prevention or delay is desired a PROX nucleic acid, a PROX polypeptide, or a PROX antibody in an amount sufficient to treat, prevent, or delay a tissue proliferation-associated disorder in the subject.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting.

Other features and advantages of the invention will be apparent from the following detailed description and claims.

### BRIEF DESCRIPTION OF THE FIGURES

FIG. 1 is an alignment of the proteins encoded by clones 17931354.0.35.1 and 17931354.0.35.2.

FIG. 2 is an alignment of the proteins encoded by Clone 7520500.0.54_{—}1; Clone 7520500.0.54 2; Clone 7520500.0.54_{—}3; Clone 7520500.0.54_{—}4; and Clone7520500.0.21.

FIG. 3 is a gel electrophoretogram showing the expression of 20468752.0.18-U protein in HEK 293 cells.

FIG. 4 is a electrophoretogram showing the expression of 11692010.0.51 protein in HEK 293 cells.

FIG. 5 is an electrophoretogram showing the expression of 27835981.0.1 protein in HEK 293 cells.

FIG. 6 is an electrophoretogram showing the expression of 21399247.0.1 protein in HEK 293 cells.

FIG. 7 is an electrophoretogram showing the expression of 17941787.0.1 protein in HEK 293 cells.

FIG. 8 is a bar graph showing inhibition of trypsin activity by the protein encoded by Clone 11692010.0.51.

FIG. 9 is a graph showing growth of NHost cells induced by the protein encoded by Clone 20468752.0.18-U.

### DETAILED DESCRIPTION OF THE INVENTION

The invention provides novel polynucleotides and the polypeptides encoded thereby. The invention is based in part on the discovery of nucleic acids encoding 17 proteins that contain sequences suggesting they are secreted, localized to a cellular organelle, or membrane associated. The invention includes 18 PROX nucleic acids, PROX polypeptides, PROX antibodies, or compounds or methods based on these nucleic acids. These nucleic acids, and their associated polypeptides, antibodies and other compositions are referred to as PRO1, PRO2, PRO3 . . . through PRO17, respectively. These sequences are collectively referred to as “PROX nucleic acids or “PROX polynucleotides” (where X is an integer between 1 and 17) and the corresponding encoded polypeptide is referred to as a “PROX polypeptide” or “PROX protein”.

Table 1 provides a cross-reference between a PROX nucleic acid or polypeptide of the invention, a table disclosing a nucleic acid and encoded polypeptide that is encompassed by an indicated PROX nucleic acid or polypeptide of the invention, and a corresponding sequence identification number (SEQ ID NO:). Also provided is a Clone Identification Number for the disclosed nucleic acid and encoded polypeptides. Unless indicated otherwise, reference to a “Clone” herein refers to a discrete in silico nucleic acid sequence.

TABLE 1

| | PROX | Table | SEQ ID NO: | SEQ ID NO: |
|---|---|---|---|---|
| Clone | Number | Number | Nucleic Acid | Polypeptide |
| 20488752.0.18 | 1 | 2 | 1 | 2 |
| 20468752.0.18-U | 2 | 3 | 3 | 4 |
| 11692010.0.51 | 3 | 4 | 5 | 6 |
| 27835981.0.1 | 4 | 5 | 7 | 8 |
| 21399247.0.1 | 5 | 6 | 9 | 10 |
| 17132296.0.4 | 6 | 7 | 11 | 12 |
| 17931354.0.35.1 | 7 | 8 | 13 | 14 |
| 17931354.0.35.2 | 8 | 9 | 15 | 16 |
| 7520500.0.54_1 | 9 | 10 | 17 | 18 |
| 7520500.0.54_2 | 10 | 11 | 19 | 20 |
| 7520500.0.54_3 | 11 | 12 | 21 | 22 |
| 7520500.0.54_4 | 12 | 13 | 23 | 23 |
| 7520500.0.21 | 13 | 14 | 25 | 26 |
| 17941787.0.1 | 14 | 15 | 27 | 28 |
| 17941787.0.31 | 15 | 16 | 29 | 30 |
| 16467945.0.85 | 16 | 17 | 31 | 32 |
| 15467945.0.88 | 17 | 19 | 33 | 34 |

PROX nucleic acids, PROX polypeptides, PROX antibodies, and related compounds, are useful in a variety of applications and contexts. For example, various PROX nucleic acids and polypeptides according to the invention are useful, inter alia, as novel members of the protein families according to the presence of domains and sequence relatedness to previously described proteins.

PROX nucleic acids and polypeptides according to the invention can also be used to identify cell types based on the presence or absence of various PROX nucleic acids according to the invention. Additional utilities for PROX nucleic acids and polypeptides are discussed below.

### PRO1 and PRO2 Nucleic Acids and Polypeptides

A PRO1 nucleic acid according to the invention includes the nucleic acid sequence represented in Clone 20468752.0.18. RNA sequences homologous to this clone are found in placenta.

A representation of the nucleotide sequence of Clone 20468752.0.18 is shown in Table 2 and includes a nucleotide sequence (SEQ ID NO:1) of 1867 bp. This nucleotide sequence has an open reading frame (ORF) encoding a polypeptide of 567 amino acid residues (SEQ ID NO:2) with a predicted molecular weight of 63327 Daltons. The start codon is located at nucleotides 128-130 and the stop codon is located at nucleotides 1829-1831. The protein encoded by Clone 20468752.0.18 (SEQ ID NO:2) was predicted by the PSORT program to be extracellularly localized with a certainty of 0.3700. Analysis using the PSORT and SignalP computer programs predicted that there is may be a signal peptide with the most likely cleavage occurring between residues 21 and 22, at the sequence ISS-LP. The nucleic acid (SEQ ID NO:1) and amino acid (SEQ ID NO:2) sequences of Clone 20468752.0.18 is shown below in Table 2.

TABLE 2

b Clone 20468752
Translated Protein—Frame: 2-Nucleotide 128 to 1828

| | | |
|---|---|---|
| 1 | GAGCTGAAACCCGAGCTCCCGCTCAGCTGGGGCTCGGGGAGGTCC | (SEQ ID NO:1) |
| 46 | CTGTAAAACCCGCCTGCCCCCGGCCTCCCTGGGTCCCTCCTCTCC | |
| 91 | CTCCCCAGTAGACGCTCGGACACCAGCCGCGGCAAGGATGGAGCT | |
| | MetGluLe | (SEQ ID NO:2) |
| 136 | GGGTTGCTGGACGCAGTTGGGGCTCACTTTTCTTCAGCTCCTTCT | |
| | uGlyCysTrpThrGlnLeuGlyLeuThrPheLeuGlnLeuLeuLe | |
| 181 | CATCTCGTCCTTGCCAAGAGAGTACACAGTCATTAATGAAGCCTG | |
| | uIleSerSerLeuProArgGluTyrThrValIleAsnGluAlaCy | |
| 226 | CCCTGGAGCAGAGTGGAATATCATGTGTCGGGAGTGCTGTGAATA | |
| | sProGlyAlaGluTrpAsnIleMetCysArgGluCysCysGluTy | |
| 271 | TGATCAGATTGAGTGCGTCTGCCCCGGAAAGAGGGAAGTCGTGGG | |
| | rAspGlnIleGluCysValCysProGlyLysArgGluValValGl | |
| 316 | TTATACCATCCCTTGCTGCAGGAATGAGGAGAATGAGTGTGACTC | |
| | yTyrThrIleProCysCysArgAsnGluGluAsnGluCysAspSe | |
| 361 | CTGCCTGATCCACCCAGGTTGTACCATCTTTGAAAACTGCAAGAG | |
| | rCysLeuIleHisProGlyCysThrIlePheGluAsnCysLysSe | |
| 406 | CTGCCGAAATGGCTCATGGGGGGGTACCTTGGATGACTTCTATGT | |
| | rCysArgAsnGlySerTrpGlyGlyThrLeuAspAspPheTyrVa | |
| 451 | GAAGGGGTTCTACTGTGCAGAGTGCCGAGCAGGCTGGTACGGAGG | |
| | lLysGlyPheTyrCysAlaGluCysArgAlaGlyTrpTyrGlyGl | |
| 496 | AGACTGCATGCGATGTGGCCAGGTTCTGCGAGCCCCAAAGGGTCA | |
| | yAspCysMetArgCysGlyGlnValLeuArgAlaProLysGlyGl | |
| 541 | GATTTTGTTGGAAAGCTATCCCCTAAATGCTCACTGTGAATGGAC | |
| | nIleLeuLeuGluSerTyrProLeuAsnAlaHisCysGluTrpTh | |
| 586 | CATTCATGCTAAACCTGGGTTTGTCATCCAACTAAGATTTGTCAT | |
| | rIleHisAlaLysProGlyPheValIleGlnLeuArgPheValMe | |
| 631 | GTTGAGCCTGGAGTTTGACTACATGTGCCAGTATGACTATGTTGA | |
| | tLeuSerLeuGluPheAspTyrMetCysGlnTyrAspTyrValGl | |
| 676 | GGTTCGTGATGGAGACAACCGCGATGGCCAGATCATCAAGCGTGT | |
| | uValArgAspGlyAspAsnArgAspGlyGlnIleIleLysArgVa | |
| 721 | CTGTGGCAACGAGCGGCCAGCTCCTATCCAGAGCATAGGATCCTC | |
| | lCysGlyAsnGluArgProAlaProIleGlnSerIleGlySerSe | |
| 766 | ACTCCACGTCCTCTTCCACTCCGATGGCTCCAAGAATTTTGACGG | |
| | rLeuHisValLeuPheHisSerAspGlySerLysAsnPheAspGl | |
| 811 | TTTCCATGCCATTTATGAGGAGATCACAGCATGCTCCTCATCCCC | |
| | yPheHisAlaIleTyrGluGluIleThrAlaCysSerSerSerPr | |
| 856 | TTGTTTCCATGACGGCACGTGCGTCCTTGACAAGGCTGGATCTTA | |
| | oCysPheHisAspGlyThrCysValLeuAspLysAlaGlySerTy | |
| 901 | CAAGTGTGCCTGCTTGGCAGGCTATACTGGGCAGCGCTGTGAAAA | |
| | rLysCysAlaCysLeuAlaGlyTyrThrGlyGlnArgCysGluAs | |
| 946 | TCTCCTTGAAGAAAGAAACTGCTCAGACCCTGGGGGCCCAGTCAA | |
| | nLeuLeuGluGluArgAsnCysSerAspProGlyGlyProValAs | |
| 991 | TGGGTACCAGAAAATAACAGGGGGCCCTGGGCTTATCAACGGACG | |
| | nGlyTyrGlnLysIleThrGlyGlyProGlyLeuIleAsnGlyAr | |
| 1036 | CCATGCTAAAATTGGCACCGTGGTGTCTTTCTTTTGTAACAACTC | |
| | gHisAlaLysIleGlyThrValValSerPhePheCysAsnAsnSe | |
| 1081 | CTATGTTCTTAGTGGCAATGAGAAAAGAACTTGCCAGCAGAATGG | |
| | rTyrValLeuSerGlyAsnGluLysArgThrCysGlnGlnAsnGl | |
| 1126 | AGAGTGGTCAGGGAAACAGCCCATCTGCATAAAAGCCTGCCGAGA | |
| | yGluTrpSerGlyLysGlnProIleCysIleLysAlaCysArgGl | |
| 1171 | ACCAAAGATTTCAGACCTGGTGAGAAGGAGAGTTCTTCCGATGCA | |
| | uProLysIleSerAspLeuValArgArgArgValLeuProMetGl | |
| 1216 | GGTTCAGTCAAGGGAGACACCATTACACCAGCTATACTCAGCGGC | |
| | nValGlnSerArgGluThrProLeuHisGlnLeuTyrSerAlaAl | |
| 1261 | CTTCAGCAAGCAGAAACTGCAGAGTGCCCCTACCAAGAAGCCAGC | |
| | aPheSerLysGlnLysLeuGlnSerAlaProThrLysLysProAl | |
| 1306 | CCTTCCCTTTGGAGATCTGCCCATGGGATACCAACATCTGCATAC | |
| | aLeuProPheGlyAspLeuProMetGlyTyrGlnHisLeuHisTh | |
| 1351 | CCAGCTCCAGTATGAGTGCATCTCACCCTTCTACCGCCGCCTGGG | |
| | rGlnLeuGlnTyrGluCysIleSerProPheTyrArgArgLeuGl | |
| 1396 | CAGCAGCAGGAGGACATGTCTGAGGACTGGGAAGTGGAGTGGGCG | |
| | ySerSerArgArgThrCysLeuArgThrGlyLysTrpSerGlYAr | |
| 1441 | GGCACCATCCTGCATCCCTATCTGCGGGAAAATTGAGAACATCAC | |
| | gAlaProSerCysIleProIleCysGlyLysIleGluAsnIleTh | |
| 1486 | TGCTCCAAAGACCCAAGGGTTGCGCTGGCCGTGGCAGGCAGCCAT | |
| | rAlaProLysThrGlnGlyLeuArgTrpProTrpGlnAlaAlaIl | |
| 1531 | CTACAGGAGGACCAGCGGGGTGCATGACGGCAGCCTACACAAGGG | |
| | eTyrArgArgThrSerGlyValHisAspGlySerLeuHisLysGl | |
| 1576 | AGCGTGGTTCCTAGTCTGCAGCGGTGCCCTGGTGAATGAGCGCAC | |
| | yAlaTrpPheLeuValCysSerGlyAlaLeuValAsnGluArgTh | |
| 1621 | TGTGGTGGTGGCTGCCCACTGTGTTACTGACCTGGGGAAGGTCAC | |
| | rValValValAlaAlaHisCysValThrAspLeuGlyLysValTh | |
| 1666 | CATGATCAAGACAGCAGACCTGAAAGTTGTTTTGGGGAAATTCTA | |
| | rMetIleLysThrAlaAspLeuLysValValLeuGlyLysPheTy | |
| 1711 | CCGGGATGATGACCGGGATGAGAAGACCATCCAGAGCCTACAGAT | |
| | rArgAspAspAspArgAspGluLysThrIleGlnSerLeuGlnIl | |
| 1756 | TTCTGCTATCATTCTGCATCCCAACTATGACCCCATCCTTGCTTT | |
| | eSerAlaIleIleLeuHisProAsnTyrAspProIleLeuAlaLe | |
| 1801 | GATGCTTGACATCGCCATCCTGAACTCCTAGACAAGGCCCGTATC | |
| | uMetLeuAspIleAlaIleLeuAsnSer | |
| 1846 | AGCACCCGAGTCCAGCCCATCT | |

The polypeptide encoded by Clone 20468752.0.18 has 562 of 565 residues (99%) identical to, and positive with a 720 residue human protein designate PRO1344 (see, PCT Publication WO 9963088-A2 published Dec. 9, 1999). In addition, it has 51 of 150 residues (34%) identical to, and 71 of 150 residues (47%) positive with the 699 residue human complement-activating component of RA-reactive factor precursor (EC 3.4.21.-) (RA-reactive factor serine protease P100) (RARF) (mannose-binding protein associated serine protease) (MASP) (ACC:P48740).

A PRO2 nucleic acid according to the invention includes the nucleic acid sequence represented in Clone 20468752.0.18-U. Sequences homologous to this clone are found in placental RNA. A representation of the nucleotide sequence of clone 20468752.0.18 is provided in Table 3 and includes a nucleotide sequence (SEQ ID NO:3) of 2306 bp.

The nucleic acid sequence of Clone 20468752.0.18-U has an open reading frame (ORF) encoding a polypeptide of 720 amino acid residues (SEQ ID NO:4) with a predicted molecular weight of 63327 Daltons. The sequence of the amino acid encoded by Clone 20468752.0.18-U is shown in Table 3. The start codon is located at nucleotides 128-130 and the stop condon is located at nucleotides 2287-2289.

The protein (SEQ ID NO:4) encoded by Clone 20468752.0.18-U is predicted by the PSORT program to extracellularly localized with a certainty of 0.3700. Analysis with the PSORT and SignalP computer programs predicted that there is may be a signal peptide, with the most likely cleavage occurring between residues 21 and 22, at the sequence ISS-LP. The nucleic acid (SEQ ID NO:3) and amino acid (SEQ ID NO:4) sequences of Clone 20468752.0.18-U is shown below in Table 3.

TABLE 3

Clone 20468752-0-18_U
Translated Protein—Frame: 2-Nucleotide 128 to 2287

| | | |
|---|---|---|
| 1 | GAGCTGAAACCCGAGCTCCCGCTCAGCTGGGGCTCGGGGAGGTCC | (SEQ ID NO:3) |
| 46 | CTGTAAAACCCGCCTGCCCCCGGCCTCCCTGGGTCCCTCCTCTCC | |
| 91 | CTCCCCAGTAGACGCTCGGACACCAGCCGCGGCAAGGATGGAGCT | |
| | MetGluLe | (SEQ ID NO:4) |
| 136 | GGGTTGCTGGACGCAGTTGGGGCTCACTTTTCTTCAGCTCCTTCT | |
| | uGlyCysTrpThrGlnLeuGlyLeuThrPheLeuGlnLeuLeuLe | |
| 181 | CATCTCGTCCTTGCCAAGAGAGTACACAGTCATTAATGAAGCCTC | |
| | uIleSerSerLeuProArgGluTyrThrValIleAsnGluAlaCy | |
| 226 | CCCTGGAGCAGAGTGGAATATCATGTGTCGGGAGTGCTGTGAATA | |
| | sProGlyAlaGluTrpAsnIleMetCysArgGluCysCysGluTy | |
| 271 | TGATCAGATTGAGTGCGTCTGCCCCGGAAAGAGGGAAGTCGTGGG | |
| | rAspGlnIleGluCysValCysProGlyLysArgGluValValGl | |
| 316 | TTATACCATCCCTTGCTGCAGGAATGAGGAGAATGAGTGTGACTC | |
| | yTyrThrIleProCysCysArgAsnGluGluAsnGluCysAspSe | |
| 361 | CTGCCTGATCCACCCAGGTTGTACCATCTTTGAAAACTGCAAGAG | |
| | rCysLeuIleHisProGlyCysThrIlePheGluAsnCysLysSe | |
| 406 | CTGCCGAAATGGCTCATGGGGGGGTACCTTGGATGACTTCTATGT | |
| | rCysArgAsnGlySerTrpGlyGlyThrLeuAspAspPheTyrVa | |
| 451 | GAAGGGGTTCTACTGTGCAGAGTGCCGAGCAGGCTGGTACGGAGG | |
| | lLysGlyPheTyrCysAlaGluCysArgAlaGlyTrpTyrGlyGl | |
| 496 | AGACTGCATGCGATGTGGCCAGGTTCTGCGAGCCCCAAAGGGTCA | |
| | yAspCysMetArgCysGlyGlnValLeuArgAlaProLysGlyGl | |
| 541 | GATTTTGTTGGAAAGCTATCCCCTAAATGCTCACTGTGAATGGAC | |
| | nIleLeuLeuGluSerTyrProLeuAsnAlaHisCysGluTrpTh | |
| 586 | CATTCATGCTAAACCTGGGTTTGTCATCCAACTAAGATTTGTCAT | |
| | rIleHisAlaLysProGlyPheValIleGlnLeuArgPheValMe | |
| 631 | GTTGAGCCTGGAGTTTGACTACATGTGCCAGTATGACTATGTTGA | |
| | tLeuSerLeuGluPheAspTyrMetCysGlnTyrAspTyrValGl | |
| 676 | GGTTCGTGATGGAGACAACCGCGATGGCCAGATCATCAAGCGTGT | |
| | uValArgAspGlyAspAsnArgAspGlyGlnIleIleLysArgVa | |
| 721 | CTGTGGCAACGAGCGGCCAGCTCCTATCCAGAGCATAGGATCCTC | |
| | lCysGlyAsnGluArgProAlaProIleGlnSerIleGlySerSe | |
| 766 | ACTCCACGTCCTCTTCCACTCCGATGGCTCCAAGAATTTTGACGG | |
| | rLeuHisValLeuPheHisSerAspGlySerLysAsnPheAspGl | |
| 811 | TTTCCATGCCATTTATGAGGAGATCACAGCATGCTCCTCATCCCC | |
| | yPheHisAlaIleTyrGluGluIleThrAlaCysSerSerSerPr | |
| 856 | TTGTTTCCATGACGGCACGTGCGTCCTTGACAAGGCTGGATCTTA | |
| | oCysPheHisAspGlyThrCysValLeuAspLysAlaGlySerTy | |
| 901 | CAAGTGTGCCTGCTTGGCAGGCTATACTGGGCAGCGCTGTGAAAA | |
| | rLysCysAlaCysLeuAlaGlyTyrThrGlyGlnArgCysGluAs | |
| 946 | TCTCCTTGAAGAAAGAAACTGCTCAGACCCTGGGGGCCCAGTCAA | |
| | nLeuLeuGluGluArgAsnCysSerAspProGlyGlyProValAs | |
| 991 | TGGGTACCAGAAAATAACAGGGGGCCCTGGGCTTATCAACGGACG | |
| | nGlyTyrGlnLysIleThrGlyGlyProGlyLeuIleAsnGlyAr | |
| 1036 | CCATGCTAAAATTGGCACCGTGGTGTCTTTCTTTTGTAACAACTC | |
| | gHisAlaLysIleGlyThrValValSerPhePheCysAsnAsnSe | |
| 1081 | CTATGTTCTTAGTGGCAATGAGAAAAGAACTTGCCAGCAGAATGG | |
| | rTyrValLeuSerGlyAsnGluLysArgThrCysGlnGlnAsnGl | |
| 1126 | AGAGTGGTCAGGGAAACAGCCCATCTGCATAAAAGCCTGCCGAGA | |
| | yGluTrpSerGlyLysGlnProIleCysIleLysAlaCysArgGl | |
| 1171 | ACCAAAGATTTCAGACCTGGTGAGAAGGAGAGTTCTTCCGATGCA | |
| | uProLysIleSerAspLeuValArgArgArgValLeuProMetGl | |
| 1216 | GGTTCAGTCAAGGGAGACACCATTACACCAGCTATACTCAGCGGC | |
| | nValGlnSerArgGluThrProLeuHisGlnLeuTyrSerAlaAl | |
| 1261 | CTTCAGCAAGCAGAAACTGCAGAGTGCCCCTACCAAGAAGCCAGC | |
| | aPheSerLysGlnLysLeuGlnSerAlaProThrLysLysProAl | |
| 1306 | CCTTCCCTTTGGAGATCTGCCCATGGGATACCAACATCTGCATAC | |
| | aLeuProPheGlyAspLeuProMetGlyTyrGlnHisLeuHisTh | |
| 1351 | CCAGCTCCAGTATGAGTGCATCTCACCCTTCTACCGCCGCCTGGG | |
| | rGlnLeuGlnTyrGluCysIleSerProPheTyrArgArgLeuGl | |
| 1396 | CAGCAGCAGGAAGACATGTCTGAAGACTGGGAAGTGGAGTGGGCG | |
| | ySerSerArgLysThrCysLeuLysThrGlyLysTrpSerGlyAr | |
| 1441 | GGCACCATCCTGCATCCCTATCTGCGGGAAAATTGAGAACATCAC | |
| | gAlaProSerCysIleProIleCysGlyLysIleGluAsnIleTh | |
| 1486 | TGCTCCAAAGACCCAAGGGTTGCGCTGGCCGTGGCAGGCAGCCAT | |
| | rAlaProLysThrGlnGlyLeuArgTrpProTrpGlnAlaAlaIl | |
| 1531 | CTACAGGAGGACCAGCGGGGTGCATGACGGCAGCCTACACAAGGG | |
| | eTyrArgArgThrSerGlyValHisAspGlySerLeuHisLysGl | |
| 1576 | AGCGTGGTTCCTAGTCTGCAGCGGTGCCCTGGTGAATGAGCGCAC | |
| | yAlaTrpPheLeuValCysSerGlyAlaLeuValAsnGluArgTh | |
| 1621 | TGTGGTGGTGGCTGCCCACTGTGTTACTGACCTGGGGAAGGTCAC | |
| | rValValValAlaAlaHisCysValThrAspLeuGlyLysValTh | |
| 1666 | CATGATCAAGACAGCAGACCTGAAAGTTGTTTTGGGGAAATTCTA | |
| | rMetIleLysThrAlaAspLeuLysValValLeuGlyLysPheTy | |
| 1711 | CCGGGATGATGACCGGGATGAGAAGACCATCCAGAGCCTACAGAT | |
| | rArgAspAspAspArgAspGluLysThrIleGlnSerLeuGlnIl | |
| 1756 | TTCTGCTATCATTCTGCATCCCAACTATGACCCCATCCTGCTTGA | |
| | eSerAlaIleIleLeuHisProAsnTyrAspProIleLeuLeuAs | |
| 1801 | TGCTGACATCGCCATCCTGAAGCTCCTAGACAAGGCCCGTATCAG | |
| | pAlaAspIleAlaIleLeuLysLeuLeuAspLysAlaArgIleSe | |
| 1846 | CACCCGAGTCCAGCCCATCTGCCTCGCTGCCAGTCGGGATCTCAG | |
| | rThrArgValGlnProIleCysLeuAlaAlaSerArgAspLeuSe | |
| 1891 | CACTTCCTTCCAGGAGTCCCACATCACTGTGGCTGGCTGGAATGT | |
| | rThrSerPheGlnGluSerHisIleThrValAlaGlyTrpAsnVa | |
| 1936 | CCTGGCAGACGTGAGGAGCCCTGGCTTCAAGAACGACACACTGCG | |
| | lLeuAlaAspValArgSerProGlyPheLysAsnAspThrLeuAr | |
| 1981 | CTCTGGGGTGGTCAGTGTGGTGGACTCGCTGCTGTGTGAGGAGCA | |
| | gSerGlyValValSerValValAspSerLeuLeuCysGluGluGl | |
| 2026 | GCATGAGGACCATGGCATCCCAGTGAGTGTCACTGATAACATGTT | |
| | nHisGluAspHisGlyIleProValSerValThrAspAsnMetPh | |
| 2071 | CTGTGCCAGCTGGGAACCCACTGCCCCTTCTGATATCTGCACTGC | |
| | eCysAlaSerTrpGluProThrAlaProSerAspIleCysThrAl | |
| 2116 | AGAGACAGGAGGCATCGCGGCTGTGTCCTTCCCGGGACGAGCATC | |
| | aGluThrGlyGlyIleAlaAlaValSerPheProGlyArgAlaSe | |
| 2161 | TCCTGAGCCACGCTGGCATCTGATGGGACTGGTCAGCTGGAGCTA | |
| | rProGluProArgTrpHisLeuMetGlyLeuValSerTrpSerTy | |
| 2206 | TGATAAAACATGCAGCCACAGGCTCTCCACTGCCTTCACCAAGGT | |
| | rAspLysThrCysSerHisArgLeuSerThrAlaPheThrLysVa | |
| 2251 | GCTGCCTTTTAAAGACTGGATTGAAAGAAATATGAAATGAACCAT | |
| | lLeuProPheLysAspTrpIleGluArgAsnMetLys | |
| 2296 | GCTCATGCACT | |

The protein encoded by Clone 20468752.0.18-U has 718 of 720 residues (99%) identical to, and 100% of 720 residues positive with, a 720 residue human protein termed PRO1344 (PCT Publication WO 9963088-A2, published Dec. 9, 1999). In addition, this encoded protein was also found to have 180 of 181 residues (99%) identical to, and 181 of 181 residues (100%) positive with, a 188 residue fragment of a hypothetical human 20.0 Kdal protein (TREMBLNEW-ACC:CAB43317).

The proteins of the invention encoded by clones 20468752.0.18 and 20468752.0.18-U include the protein disclosed as being encoded by the ORFs described herein, as well as any mature protein arising therefrom as a result of post-translational modifications. Thus, the proteins of the invention encompass both a precursor and any active forms of the 20468752.0.18 and 20468752.0.18-U proteins.

Experimental results shown in Example 16 have shown that Clone 20468752 is relatively strongly expressed in certain central nervous system tumors and melanomas; and suppressed in most colon cancer, breast cancer, ovarian cancer, prostate cancer, lung cancer, and liver cancer samples, in comparison to the respective normal cell samples from the same tissues. These results suggest that the nucleic acid or amino acid sequences clone may be useful in the detection, diagnosis, or treatment of these cancers. Furthermore, results shown in Examples 17 indicate that expression of this nucleic acid sequence also induces growth of NHost cells.

### PRO3

A PRO3 nucleic acid according to the invention includes the nucleic acid sequence represented in Clone 11692010.0.51. RNA sequences homologous to this clone are found in fetal brain tissue. A representation of the nucleotide sequence of Clone 11692010.0.51 is provided in Table 4 and includes a nucleotide sequence (SEQ ID NO:5) of 2852 bp. This nucleotide sequence has an open reading frame (ORF) encoding a polypeptide of 649 amino acid residues (SEQ ID NO:6) with a predicted molecular weight of 72993.5 Daltons. The start codon is located at nucleotides 458460 and the stop codon is located at nucleotides 2405-2407. The protein (SEQ ID NO:6) was predicted by the PSORT computer program to be localized to the plasma membrane with a certainty of 0.6976. The SignalP computer program predicted that there is a signal peptide, with the most likely cleavage site occurring between residues 28 and 29, at the sequence VMA-KS. The nucleic acid (SEQ ID NO:5) and amino acid (SEQ ID NO:6) sequences of Clone 11692010.0.51 are shown below in Table 4.

TABLE 4

Clone 11692010-0-51
Translated Protein—Frame: 2-Nucleotide 458 to 2404

| | | |
|---|---|---|
| 1 | GTGTGCAGTAAACTGGAATGCTCTCCCTCGCTCACTCCTCAGTGT | (SEQ ID NO:5) |
| 46 | AGGAGTGATCTGAAGCAGGACAAGCTCAGCCTGCAGCTGCCGTGG | |
| 91 | GCTTTGTGTGGACTGGACGCAGAGGTTGGGAGACGGGGGAGGGCT | |
| 136 | ATTACTCCAATTCACTGTCAATGGAATTACAGCTATAGCGGCAGT | |
| 181 | GTATATAGGATTGCTTTTTCTCGTCTTCCTGGAGATGCTCAGTCC | |
| 226 | CAGTATATTTTAAGGAAGAGAAATATAAAGGAAATTTAGTATGCC | |
| 271 | TCCTTTTCTTTAAATGAAGAATTTAGTTTCCTTTACTTCTTAAAA | |
| 316 | GAGAATACCTGTTCTTGTATAACGTGACTGCACCAGACATTCTGA | |
| 361 | AAAATCAGCAAGAAGCAAAAGCTGGAAATAGCTATTTCACAGCAG | |
| 406 | GGTTCTGAAGTAACGGAAGCTACCTTGTATAAAGACCTCAACACT | |
| 451 | GCTGACCATGATCAGCGCAGCCTGGAGCATCTTCCTCATCGGGAC | |
| | MetIleSerAlaAlaTrpSerIlePheLeuIleGlyTh | (SEQ ID NO:6) |
| 496 | TAAAATTGGGCTGTTCCTTCAAGTAGCACCTCTATCAGTTATGGC | |
| | rLysIleGlyLeuPheLeuGlnValAlaProLeuSerValMetAl | |
| 541 | TAAATCCTGTCCATCTGTGTGTCGCTGCGATGCGGGTTTCATTTA | |
| | aLysSerCysProSerValCysArgCysAspAlaGlyPheIleTy | |
| 586 | CTGTAATGATCGCTTTCTGACATCCATTCCAACAGGAATACCAGA | |
| | rCysAsnAspArgPheLeuThrSerIleProThrGlyIleProGl | |
| 631 | GGATGCTACAACTCTCTACCTTCAGAACAACCAAATAAATAATGC | |
| | uAspAlaThrThrLeuTyrLeuGlnAsnAsnGlnIleAsnAsnAl | |
| 676 | TGGGATTCCTTCAGATTTGAAAAACTTGCTGAAAGTAGAAAGAAT | |
| | aGlyIleProSerAspLeuLysAsnLeuLeuLysValGluArgIl | |
| 721 | ATACCTATACCACAACAGTTTAGATGAATTTCCTACCAACCTCCC | |
| | eTyrLeuTyrHisAsnSerLeuAspGluPheProThrAsnLeuPr | |
| 766 | AAAGTATGTAAAAGAGTTACATTTGCAAGAAAATAACATAAGGAC | |
| | oLysTyrValLysGluLeuHisLeuGlnGluAsnAsnIleArgTh | |
| 811 | TATCACTTATGATTCACTTTCAAAAATTCCCTATCTGGAAGAATT | |
| | rIleThrTyrAspSerLeuSerLysIleProTyrLeuGluGluLe | |
| 856 | ACATTTAGATGACAACTCTGTCTCTGCAGTTAGCATAGAAGAGGG | |
| | uHisLeuAspAspAsnSerValSerAlaValSerIleGluGluGl | |
| 901 | AGCATTCCGAGACAGCAACTATCTCCGACTGCTTTTCCTGTCCCG | |
| | yAlaPheArgAspSerAsnTyrLeuArgLeuLeuPheLeuSerAr | |
| 946 | TAATCACCTTAGCACAATTCCCTGGGGTTTGCCCAGGACTATAGA | |
| | gAsnHisLeuSerThrIleProTrpGlyLeuProArgThrIleGl | |
| 991 | AGAACTACGCTTGGATGATAATCGCATATCCACTATTTCATCACC | |
| | uGluLeuArgLeuAspAspAsnArgIleSerThrIleSerSerPr | |
| 1036 | ATCTCTTCAAGGTCTCACTAGTCTAAAACGCCTGGTTCTAGATGG | |
| | oSerLeuGlnGlyLeuThrSerLeuLysArgLeuValLeuAspGl | |
| 1081 | AAACCTGTTGAACAATCATGGTTTAGGTGACAAAGTTTTCTTCAA | |
| | yAsnLeuLeuAsnAsnHisGlyLeuGlyAspLysValPhePheAs | |
| 1126 | CCTAGTTAATTTGACAGAGCTGTCCCTGGTGCGGAATTCCCTGAC | |
| | nLeuValAsnLeuThrGluLeuSerLeuValArgAsnSerLeuTh | |
| 1171 | TGCTGCACCAGTAAACCTTCCAGGCACAAACCTGAGGAAGCTTTA | |
| | rAlaAlaProValAsnLeuProGlyThrAsnLeuArgLysLeuTy | |
| 1216 | TCTTCAAGATAACCACATCAATCGGGTGCCCCCAAATGCTTTTTC | |
| | rLeuGlnAspAsnHisIleAsnArgValProProAsnAlaPheSe | |
| 1261 | TTATCTAAGGCAGCTCTATCGACTGGATATGTCCAATAATAACCT | |
| | rTyrLeuArgGlnLeuTyrArgLeuAspMetSerAsnAsnAsnLe | |
| 1306 | AAGTAATTTACCTCAGGGTATCTTTGATGATTTGGACAATATAAC | |
| | uSerAsnLeuProGlnGlyIlePheAspAspLeuAspAsnIleTh | |
| 1351 | ACAACTGATTCTTCGCAACAATCCCTGGTATTGCGGGTGCAAGAT | |
| | rGlnLeuIleLeuArgAsnAsnProTrpTyrCysGlyCysLysMe | |
| 1396 | GAAATGGGTACGTGACTGGTTACAATCACTACCTGTGAAGGTCAA | |
| | tLysTrpValArgAspTrpLeuGlnSerLeuProValLysValAs | |
| 1441 | CGTGCGTGGGCTCATGTGCCAAGCCCCAGAAAAGGTTCGTGGGAT | |
| | nValArgGlyLeuMetCysGlnAlaProGluLysValArgGlyMe | |
| 1486 | GGCTATTAAGGATCTCAATGCAGAACTGTTTGATTGTAAGGACAG | |
| | tAlaIleLysAspLeuAsnAlaGluLeuPheAspCysLysAspSe | |
| 1531 | TGGGATTGTAAGCACCATTCAGATAACCACTGCAATACCCAACAC | |
| | rGlyIleValSerThrIleGlnIleThrThrAlaIleProAsnTh | |
| 1576 | AGTGTATCCTGCCCAAGGACAGTGGCCAGCTCCAGTGACCAAACA | |
| | rValTyrProAlaGlnGlyGlnTrpProAlaProValThrLysGl | |
| 1621 | GCCAGATATTAAGAACCCCAAGCTCACTAAGGATCAACAAACCAC | |
| | nProAspIleLysAsnProLysLeuThrLysAspGlnGlnThrTh | |
| 1666 | AGGGAGTCCCTCAAGAAAAACAATTACAATTACTGTGAAGTCTGT | |
| | rGlySerProSerArgLysThrIleThrIleThrValLysSerVa | |
| 1711 | CACCTCTGATACCATTCATATCTCTTGGAAACTTGCTCTACCTAT | |
| | lThrSerAspThrIleHisIleSerTrpLysLeuAlaLeuProMe | |
| 1756 | GACTGCTTTGAGACTCAGCTGGCTTAAACTGGGCCATAGCCCGGC | |
| | tThrAlaLeuArgLeuSerTrpLeuLysLeuGlyHisSerProAl | |
| 1801 | ATTTGGATCTATAACAGAAACAATTGTAACAGGGGAACGCAGTGA | |
| | aPheGlySerIleThrGluThrIleValThrGlyGluArgSerGl | |
| 1846 | GTACTTGGTCACAGCCCTGGAGCCTGATTCACCCTATAAAGTATG | |
| | uTyrLeuValThrAlaLeuGluProAspSerProTyrLysValCy | |
| 1891 | CATGGTTCCCATGGAAACCAGCAACCTCTACCTATTTGATGAAAC | |
| | sMetValProMetGluThrSerAsnLeuTyrLeuPheAspGluTh | |
| 1936 | TCCTGTTTGTATTGAGACTGAAACTGCACCCCTTCGAATGTACAA | |
| | rProValCysIleGluThrGluThrAlaProLeuArgMetTyrAs | |
| 1981 | CCCTACAACCACCCTCAATCGAGAGCAAGAGAAAGAACCTTACAA | |
| | nProThrThrThrLeuAsnArgGluGlnGluLysGluProTyrLy | |
| 2026 | AAACCCCAATTTACCTTTGGCTGCCATCATTGGTGGGGCTGTGGC | |
| | sAsnProAsnLeuProLeuAlaAlaIleIleGlyGlyAlaValAl | |
| 2071 | CCTGGTTACCATTGCCCTTCTTGCTTTAGTGTGTTGGTATGTTCA | |
| | aLeuValThrIIeAlaLeuLeuAlaLeuValCysTrpTyrValHi | |
| 2116 | TAGGAATGGATCGCTCTTCTCAAGGAACTGTGCATATAGCAAAGG | |
| | sArgAsnGlySerLeuPheSerArgAsnCysAlaTyrSerLysGl | |
| 2161 | GAGGAGAAGAAAGGATGACTATGCAGAAGCTGGCACTAAGAAGGA | |
| | yArgArgArgLysAspAspTyrAlaGluAlaGlyThrLysLysAs | |
| 2206 | CAACTCTATCCTGGAAATCAGGGAAACTTCTTTTCAGATGTTACC | |
| | pAsnSerIleLeuGluIleArgGluThrSerPheGlnMetLeuPr | |
| 2251 | AATAAGCAATGAACCCATCTCGAAGGAGGAGTTTGTAATACACAC | |
| | oIleSerAsnGluProIleSerLysGluGluPheValIleHisTh | |
| 2296 | CATATTTCCTCCTAATGGAATGAATCTGTACAAAAACAATCACAG | |
| | rIlePheProProAsnGlyMetAsnLeuTyrLysAsnAsnHisSe | |
| 2341 | TGAAAGCAGTAGTAACCGAAGCTACAGAGACAGTGGTATTCCAGA | |
| | rGluSerSerSerAsnArgSerTyrArgAspSerGlyIleProAs | |
| 2386 | CTCAGATCACTCACACTCATGATGCTGAAGGACTCACAGCAGACT | |
| | pSerAspHisSerHisSer | |
| 2431 | TGTGTTTTGGGTTTTTTAAACCTAAGGGAGGTGATGGTAGGAACC | |
| 2476 | CTGTTCTACTGCAAAACACTGGAAAAAGAGACTGAAAAAAAGCAA | |
| 2521 | TGTACTGTACATTTGCCATATAATTTATATTTAAGAACTTTTTAT | |
| 2566 | TAAAAGTTTCAAATTTCAGGTTACTGCTGCGATTGATGTAGTGGA | |
| 2611 | GATGCCTGAACACAATTCTATATTTTAGTATTTTTTAGTAATTTG | |
| 2656 | TACTGTATTTTCCTTGCAAATATTGGAGTTATAAACCATTTACTT | |
| 2701 | TGTGTTCTACTGAGTAAGATGACTTGTTGACTGTGAAAGTGAATT | |
| 2746 | TTCTTGCTGTGTCGAACAATCAGGACTGCATTCATATGAGATCCT | |
| 2791 | TGTAGTATAAGCACAGGCCATTTTTCACTTTGGTATTAATAAAAT | |
| 2836 | GTAAAAAAAAAATTGGT | |

BLAST P and BLASTX analyses indicate that the protein encoded by Clone 11692010.0.51 has 306 of 637 residues (48%) identical to, and 427 of 637 residues (67%) positive with, a 660 residue human KIAA0405 protein (ACC:043155). In addition, the protein encoded by Clone 11692010.0.51 was also found to have 626 of 649 residues (96%) identical to, and 637 of 649 residues (98%) positive with, the 649 residue mouse skin cell protein designated SEQ ID NO:305 (see, PCT Publication WO 9955865-A1; published Nov. 4, 1999).

The protein encoded by Clone 11692010.0.51 (SEQ ID NO:6) may potentially be used to: (i) stimulate the growth and motility of keratinocytes; (ii) to inhibit the growth of cancer cells, including melanomas; (iii) to modulate angiogenesis and tumor vascularisation,; (iv) to modulate skin inflammation; and (v) to modulate epithelial cell growth.

The proteins of the invention encoded by Clone 11692010.0.51 include the protein disclosed as being encoded by the ORFs described herein, as well as any mature protein arising therefrom as a result of post-translational modifications. Thus, the proteins of the invention encompass both a precursor and any active forms of the 11692010.0.51 protein.

Experimental results presented in Example 16 demonstrate that amino acid sequence encoded by Clone 11692010.0.51 shows high levels of expression (relative to normal cells) in certain ovarian cancer cell lines, in gastric cancer, and in a colon cancer cell line. In addition, the amino acid sequence encoded by Clone 11692010.0.51 is also found to be broadly expressed in various lung cancers and certain CNS cancer cells. These results suggest that this clone may be used as a selective probe for detection or diagnosis of these cancers, and that the clones or their gene products may be useful therapeutics or targets in treatment of such cancers. In addition, this gene product has been shown in Example 17 to inhibit serine protease activity. This property may make it useful in modulating tissue remodeling or in treating certain cancers.

### PRO4

A PRO4 nucleic acid according to the invention includes the nucleic acid sequence represented in Clone 27835981.0.1. RNA sequences homologous to this clone are found in the pancreas.

A representation of the nucleotide sequence of Clone 27835981.0.1 is illustrated in Table 5 and includes a nucleotide sequence (SEQ ID NO:7) of 1653 bp. The nucleotide sequence of Clone 27835981.0.1 has an open reading frame (ORF) encoding a polypeptide of 160 amino acid residues (SEQ ID NO:8) with a predicted molecular weight of 17844.2 Daltons. The start codon is located at nucleotides 964-966 and the stop codon is located at nucleotides 1444-1446. The protein (SEQ ID NO:8) was predicted by the PSORT computer program to be extracellularly localized with a certainty of 0. 6090. The SignalP computer program predicted that there is a signal peptide, with the most likely cleavage site located between residues 24 and 25: at the sequence TMA-EA. The nucleic acid (SEQ ID NO:7) and amino acid (SEQ ID NO:8) sequences of Clone 27835981.0.1 are shown below in Table 5.

TABLE 5

Clone 27835981.0.1
Translated Protein—Frame: 1-Nucleotide 964 to 1443

| | | |
|---|---|---|
| 1 | CCCACGCGTCCGGCCTTCTCTCTGGACTTTGCATTTCCATTCCTT | (SEQ ID NO:7) |
| 46 | TTCATTGACAAACTGACTTTTTTTATTTCTTTTTTTCCATCTCTG | |
| 91 | GGCCAGCTTGGGATCCTAGGCCGCCCTGGGAAGACATTTGTGTTT | |
| 136 | TACACACATAAGGATCTGTGTTTGGGGTTTCTTCTTCCTCCCCTG | |
| 181 | ACATTGGCATTGCTTAGTGGTTGTGTGGGGAGGGAGACCACGTGG | |
| 226 | GCTCAGTGCTTGCTTGCACTTATCTGCCTAGGTACATCGAAGTCT | |
| 271 | TTTGACCTCCATACAGTGATTATGCCTGTCATCGCTGGTGGTATC | |
| 316 | CTGGCGGCCTTGCTCCTGCTGATAGTTGTCGTGCTCTGTCTTTAC | |
| 361 | TTCAAAATACACAACGCGCTAAAAGCTGCAAAGGAACCTGAAGCT | |
| 406 | GTGGCTGTAAAAAATCACAACCCAGACAAGGTGTGGTGGGCCAAG | |
| 451 | AACAGCCAGGCCAAAACCATTGCCACGGAGTCTTGTCCTGCCCTG | |
| 496 | CAGTGCTGTGAAGGATATAGAATGTGTGCCAGTTTTGATTCCCTG | |
| 541 | CCACCTTGCTGTTGCGACATAAATGAGGGCCTCTGAGTTAGGAAA | |
| 586 | GGCTCCCTTCTCAAAGCAGAGCCCTGAAGACTTCAATGATGTCAA | |
| 631 | TGAGGCCACCTGTTTGTGATGTGCAGGCACAGAAGAAAGGCACAG | |
| 676 | CTCCCCATCAGTTTCATGGAAAATAACTCAGTGCCTGCTGGGAAC | |
| 721 | CAGCTGCTGGAGATCCCTACAGAGAGCTTCCACTGGGGGCAACCC | |
| 766 | TTCCAGGAAGGAGTTGGGGAGAGAGAACCCTCACTGTGGGGAATG | |
| 811 | CTGATAAACCAGTCACACAGCTGCTCTATTCTCACACAAATCTAC | |
| 856 | CCCTTGCGTGGCTGGAACTGACGTTTCCCTGGAGGTGTCCAGAAA | |
| 901 | GCTGATGTAACACAGAGCCTATAAAAGCTGTCGGTCCTTAAGGCT | |
| 946 | GCCCAGCGCCTTGCCAAAATGGAGCTTGTAAGAAGGCTCATGCCA | |
| | MetGluLeuValArgArgLeuMetPro | (SEQ ID NO:8) |
| 991 | TTGACCCTCTTAATTCTCTCCTGTTTGGCGGAGCTGACAATGGCG | |
| | LeuThrLeuLeuIleLeuSerCysLeuAlaGluLeuThrMetAla | |
| 1036 | GAGGCTGAAGGCAATGCAAGCTGCACAGTCAGTCTAGGGGGTGCC | |
| | GluAlaGluGlyAsnAlaSerCysThrValSerLeuGlyGlyAla | |
| 1081 | AATATGGCAGAGACCCACAAAGCCATGATCCTGCAACTCAATCCC | |
| | AsnMetAlaGluThrHisLysAlaMetIleLeuGlnLeuAsnPro | |
| 1126 | AGTGAGAACTGCACCTGGACAATAGAAAGACCAGAAAACAAAAGC | |
| | SerGluAsnCysThrTrpThrIleGluArgProGluAsnLysSer | |
| 1171 | ATCAGAATTATCTTTTCCTATGTCCAGCTTGATCCAGATGGAAGC | |
| | IleArgIleIlePheSerTyrValGlnLeuAspProAspGlySer | |
| 1216 | TGTGAAAGTGAAAACATTAAAGTCTTTGACGGAACCTCCAGCAAT | |
| | CysGluSerGluAsnIleLysValPheAspGlyThrSerSerAsn | |
| 1261 | GGGCCTCTGCTAGGGCAAGTCTGCAGTAAAAACGACTATGTTCCT | |
| | GlyProLeuLeuGlyGlnValCysSerLysAsnAspTyrValPro | |
| 1306 | GTATTTGAATCATCATCCAGTACATTGACGTTTCAAATAGTTACT | |
| | ValPheGluSerSerSerSerThrLeuThrPheGlnIleValThr | |
| 1351 | GACTCAGCAAGAATTCAAAGAACTGTCTTTGTCTTCTACTACTTC | |
| | AspSerAlaArgIleGlnArgThrValPheValPheTyrTyrPhe | |
| 1396 | TTCTCTCCTAACATCTGGCTCTGCATTCACAGCACCTACATTCCA | |
| | PheSerProAsnIleTrpLeuCysIleHisSerThrTyrIlePro | |
| 1441 | CTGTGATCCGAAGCAGAATGCCAAGAACATCTGCGAGTGGGTTCA | |
| | Leu | |
| 1486 | TGAGGAGAGCTCCACTGTGGATTTCTTTCCAAGGCCCAGAGCTGA | |
| 1531 | CCATGTCACTCTCCTGCTAAAACCACTGACTTCTTGGTACCAGCA | |
| 1576 | GATCTCCAGAGTGCAGCAGTCAAGGTTTTCCCACGCTGGACCCAG | |
| 1621 | GCCCTGTCCCATCAAAAAAAAAAAAAAAAAAAA | |

Analysis of the sequence databases using the BLAST P and BLASTX computer programs revealed that the protein encoded by Clone 27835981.0.1 has 99 of 146 residues (67%) identical to, and 120 of 146 residues (82%) positive with, a 607 residue rat uterus/ovary-specific putative transmembrane protein (ACC:Q35360). In addition, the encoded protein was also found to have residues 1-149 100% identical to the amino-terminus of a 607 amino acid residue human pancreatic PA153 consensus protein (PCT Publication WO 9931274-A2, published Jun. 24, 1999), as well as having the same 100% identity to a human protein PRO257 comprising 607 amino acid residues (PCT Publication WO 9914328-A2, published Mar. 25, 1999).

The proteins of the invention encoded by clone 27835981.0.1 include the protein disclosed as being encoded by the ORFs described herein, as well as any mature protein arising therefrom as a result of post-translational modifications. Thus, the proteins of the invention encompass both a precursor and any active forms of the 27835981.0.1 protein.

Experimental results presented in Example 16 showed that Clone 27835981.0.1 was over-expressed in virtually all cancer cell lines examined, relative to the respective normal cell lines for the same tissues. These results suggest that this clone may be used as a selective probe for detection or diagnosis of these cancers, and that the clones or their gene products may be useful therapeutics or targets in treatment of such cancers.

### PRO5 Nucleic Acids and Polypeptides

A PRO5 nucleic acid according to the invention includes the nucleic acid sequence represented in Clone 21399247.0.1. RNA sequences homologous to this clone are found in thyroid gland. A representation of the nucleotide sequence of clone 21399247.0.1 is given in Table 6 and includes a nucleotide sequence (SEQ ID NO:9) of 2478 bp. The nucleotide sequence of Clone 21399247.0.1 has an open reading frame (ORF) encoding a polypeptide of 580 amino acid residues (SEQ ID NO:10) with a predicted molecular weight of 66614.6 Daltons. The start codon is located at nucleotides 273-275 and the stop codon is located at nucleotides 2013-2015. The protein (SEQ ID NO:10) encoded by Clone 21399247.0.1 was predicted by the PSORT computer program to be localized in the microsome (lumen) with a certainty of 0.8650. The PSORT and SignalP computer programs also predicted that there is a signal peptide, with the most likely cleavage site located between residues 16 and 17, at the sequence VLA-AV. The nucleic acid (SEQ ID NO:9) and amino acid (SEQ ID NO:10) sequences OF Clone 21399247.0.1 are shown below in Table 6.

TABLE 6

Clone 21399247.0.1
Translated Protein—Frame: 3-Nucleotide 273 to 2012

| | | |
|---|---|---|
| 1 | CCGCGTCGGCAGAGGTGGCTTCGTCCCGCGGAGTCCAGGCTTCAG | (SEQ ID NO:9) |
| 46 | CTCCTGGCTTCTCTTCTTTCCTCCTAGAGATCAGATGTCGGAACT | |
| 91 | CCAGCTGAGGGCATGTCTTACTGGGCACGCAGGTGTCCTCTCTTG | |
| 136 | AGAAGAACTGTCCATACCATGGTGGTGGTAAGGCTTTCACCAGTT | |
| 181 | CTCAGGATGCCCATAGGGATGGGTGAAGCCTGCCTGGCCTGTGGT | |
| 226 | GCTTTCCAGTGGCCGTCATCTCATTAGGGCCCCACAGTGGCATTA | |
| 271 | GGATGCACCTCTCGGCGGTGTTCAACGCCCTCCTGGTGTCGGTGC | |
| | MetHisLeuSerAlaValPheAsnAlaLeuLeuValSerValL | (SEQ ID NO:10) |
| 316 | TGGCAGCGGTCCTGTGGAAGCATGTGCGGCTGCGTGAGCATGCAG | |
| | euAlaAlaValLeuTrpLysHisValArgLeuArgGluHisAlaA | |
| 361 | CCACACTGGAGGAGGAGCTGGCCCTCAGCCGACAGGCCACAGAGC | |
| | laThrLeuGluGluGluLeuAlaLeuSerArgGlnAlaThrGluP | |
| 406 | CAGCCCCAGCACTGAGGATCGACTACCCGAAGGCACTGCAGATCC | |
| | roAlaProAlaLeuArgIleAspTyrProLysAlaLeuGlnIleL | |
| 451 | TGATGGAGGGCGGCACACACATGGTGTGCACGGGCCGCACGCACA | |
| | euMetGluGlyGlyThrHisMetValCysThrGlyArgThrHisT | |
| 496 | CAGACCGCATCTGCCGCTTCAAGTGGCTCTGCTACTCCAACGAGG | |
| | hrAspArgIleCysArgPheLysTrpLeuCysTyrSerAsnGluA | |
| 541 | CTGAGGAGTTCATCTTCTTCCATGGCAACACCTCTGTCATGCTGC | |
| | laGluGluPheIlePhePheHisGlyAsnThrSerValMetLeuP | |
| 586 | CCAACCTGGGCTCCCGGCGCTTCCAGCCAGCCCTGCTCGACCTAT | |
| | roAsnLeuGlySerArgArgPheGlnProAlaLeuLeuAspLeuS | |
| 631 | CCACCGTGGAGGACCACAACACTCAGTACTTCAACTTCGTGGAGC | |
| | erThrValGluAspHisAsnThrGlnTyrPheAsnPheValGluL | |
| 676 | TGCCTGCTGCTGCCCTGCGCTTCATGCCCAAGCCGGTGTTCGTGC | |
| | euProAlaAlaAlaLeuArgPheMetProLysProValPheValP | |
| 721 | CAGACGTGGCCCTCATCGCCAACCGCTTCAACCCCGACAACCTCA | |
| | roAspValAlaLeuIleAlaAsnArgPheAsnProAspAsnLeuM | |
| 766 | TGCACGTCTTTCATGACGACCTGCTGCCACTCTTCTACACCCTGC | |
| | etHisValPheHisAspAspLeuLeuProLeuPheTyrThrLeuA | |
| 811 | GGCAGTTTCCCGGCCTGGCCCACGAGGCACGGCTCTTCTTCATGG | |
| | rgGlnPheProGlyLeuAlaHisGluAlaArgLeuPhePheMetG | |
| 856 | AGGGCTGGGGCGAGGGTGCACACTTCGACCTCTACAAGCTGCTCA | |
| | luGlyTrpGlyGluGlyAlaHisPheAspLeuTyrLysLeuLeuS | |
| 901 | GCCCCAAGCAGCCTCTCCTGCGGGCACAGCTGAAGACCCTGGGCC | |
| | erProLysGlnProLeuLeuArgAlaGlnLeuLysThrLeuGlyA | |
| 946 | GGCTGCTGTGCTTCTCCCATGCTTTTGTGGGCCTCTCCAAGATCA | |
| | rgLeuLeuCysPheSerHisAlaPheValGlyLeuSerLysIleT | |
| 991 | CTACCTGGTACCAGTATGGCTTTGTGCAGCCCCAGGGCCCGAAGG | |
| | hrThrTrpTyrGlnTyrGlyPheValGlnProGlnGlyProLysA | |
| 1036 | CCAACATCCTCGTCTCAGGCAATGAGATCCGGCAGTTTGCACGGT | |
| | laAsnIleLeuValSerGlyAsnGluIleArgGlnPheAlaArgP | |
| 1081 | TCATGACAGAAAAGCTGAACGTGAGCCACACAGGAGTCCCCCTAG | |
| | heMetThrGluLysLeuAsnValSerHisThrGlyValProLeuG | |
| 1126 | GCGAGGAGTACATTCTGGTCTTTAGCCGAACCCAGAACAGACTCA | |
| | lyGluGluTyrIleLeuValPheSerArgThrGlnAsnArgLeuI | |
| 1171 | TTCTGAATGAGGCAGAGCTGCTGCTGGCACTGGCCCAGGAGTTCC | |
| | leLeuAsnGluAlaGluLeuLeuLeuAlaLeuAlaGlnGluPheG | |
| 1216 | AGATGAAGACAGTGACAGTGTCCCTGGAGGACCACACCTTTGCTG | |
| | lnMetLysThrValThrValSerLeuGluAspHisThrPheAlaA | |
| 1261 | ATGTCGTGCGGCTGGTCAGCAATGCCTCCATGCTGGTCAGCATGC | |
| | spValValArgLeuValSerAsnAlaSerMetLeuValSerMetH | |
| 1306 | ATGGGGCCCAGCTGGTCACCACCCTCTTCCTGCCCCGTGGGGCAA | |
| | isGlyAlaGlnLeuValThrThrLeuPheLeuProArgGlyAlaT | |
| 1351 | CTGTGGTAGAGCTCTTCCCATATGCTGTCAATCCCGACCACTACA | |
| | hrValValGluLeuPheProTyrAlaValAsnProAspHisTyrT | |
| 1396 | CTCCCTATAAGACGCTGGCCATGCTGCCTGGCATGGACCTCCAGT | |
| | hrProTyrLysThrLeuAlaMetLeuProGlyMetAspLeuGlnT | |
| 1441 | ATGTAGCCTGGCGGAACATGATGCCAGAGAACACAGTCACACACC | |
| | yrValAlaTrpArgAsnMetMetProGluAsnThrValThrHisP | |
| 1486 | CTGAGCGGCCCTGGGATCAGGGGGGCATCACCCATCTGGACCGGG | |
| | roGluArgProTrpAspGlnGlyGlyIleThrHisLeuAspArgA | |
| 1531 | CTGAGCAAGCCCGTATCCTGCAAAGCCGTGAGGTCCCACGGCATC | |
| | laGluGlnAlaArgIleLeuGlnSerArgGluValProArgHisL | |
| 1576 | TCTGTTGCCGGAACCCCGAGTGGCTCTTCCGAATCTACCAGGACA | |
| | euCysCysArgAsnProGluTrpLeuPheArgIleTyrGlnAspT | |
| 1621 | CCAAGGTGGACATCCCATCCCTCATTCAAACCATACGGCGCGTGG | |
| | hrLysValAspIleProSerLeuIleGlnThrIleArgArgValV | |
| 1666 | TGAAGGGCCGGCCAGGACCACGGAAGCAGAAGTGGACAGTCGGCC | |
| | alLysGlyArgProGlyProArgLysGlnLysTrpThrValGlyL | |
| 1711 | TATATCCAGGCAAGGTGCGGGAGGCACGGTGCCAGGCGTCAGTGC | |
| | euTyrProGlyLysValArgGluAlaArgCysGlnAlaSerValH | |
| 1756 | ATGGCGCCTCCGAGGCCCGCCTCACTGTCTCCTGGCAGATCCCAT | |
| | isGlyAlaSerGluAlaArgLeuThrValSerTrpGlnIleProT | |
| 1801 | GGAACCTTAAATACCTGAAGGTGAGGGAGGTGAAGTACGAGGTGT | |
| | rpAsnLeuLysTyrLeuLysValArgGluValLysTyrGluValT | |
| 1846 | GGCTGCAGGAGCAGGGGGAGAACACCTACGTGCCTTACATCCTGG | |
| | rpLeuGlnGluGlnGlyGluAsnThrTyrValProTyrIleLeuA | |
| 1891 | CTCTGCAGAACCACACCTTCACTGAGAACATCAAGCCCTTCACCA | |
| | laLeuGlnAsnHisThrPheThrGluAsnIleLysProPheThrT | |
| 1936 | CCTACCTGGTGTGGGTCCGCTGCATCTTCAACAAGATCCTCCTGG | |
| | hrTyrLeuValTrpValArgCysIlePheAsnLysIleLeuLeuG | |
| 1981 | GACCCTTTGCAGATGTGCTGGTGTGCAACACGTAGCGAGCAGGCC | |
| | lyProPheAlaAspValLeuValCysAsnThr | |
| 2026 | ACAGCCTGGCCTCGGGAAGGTGGCTCCTGCAGTTCAGCGTCCCTG | |
| 2071 | GGCCCATTAATCCCACTGTGGAGACTTCTGGGAACTATTTATTGA | |
| 2116 | GCAGGCCTGTGCCTCCACATCATCTTGTTGTCTCTGGGGTGTGGT | |
| 2161 | GTCACAGCACTCCTCTTTGCCCTAGAGATAAGGGACCTGACTTCC | |
| 2206 | CCTTCTCCCATCCTGAACATTTGTACCCCTGGAGAAGTTCCTTAG | |
| 2251 | CAGGGAGGAGGAAGAGGAGAGGAGGAAGCAAAGAATCACAAGGAA | |
| 2296 | CCTCTGGCTAGGTGATCCTGATGTTTCCTACTGAGTTTTTCTGGT | |
| 2341 | ATCCAGATTTCTGGAAACCGCGTAATCATGTACTGTTTGATTGGG | |
| 2386 | TGGTTCATCTGCTTCCATCCCAGTGAAATTTACCTGTAGCCCAGT | |
| 2431 | GAAGGGTGTGTTTGGAACATTCATTAAATGATTCTAAGCGAAAAA | |
| 2476 | AAA | |

A search of the sequence databases using BLAST P and BLASTX reveals no statistically significant similarity to any known animal protein.

The proteins of the invention encoded by clone 21399247.0.1 include the protein disclosed as being encoded by the ORFs described herein, as well as any mature protein arising therefrom as a result of post-translational modifications. Thus, the proteins of the invention encompass both a precursor and any active forms of the 21399247.0.1 protein.

Experimental results presented in Example 16 show that clone 21399247.0.1 is broadly expressed in most of the tissues examined. Specifically, it was found to be particularly strongly expressed in certain cancers (eg., melanoma, prostate cancer, lung cancer and colon cancer). These results suggest that this clone may be used as a selective probe for detection or diagnosis of these cancers, and that the clones or their gene products may be useful therapeutics or targets in treatment of such cancers.

### PRO6

A PRO6 nucleic acid according to the invention includes the nucleic acid sequence represented in the nucleic acid sequence represented in Clone 17132296.0.4. RNA sequences homologous to this clone are found in the testis. A representation of the nucleotide sequence of Clone 17132296.0.4 is presented in Table 7 and includes a nucleotide sequence (SEQ ID NO:11) of 523 bp. This nucleotide sequence has an open reading frame (ORF) encoding a polypeptide of 121 amino acid residues (SEQ ID NO:12) with a predicted molecular weight of 13132 Daltons. The start codon is located at nucleotides 141-143 and the stop codon is located at nucleotides 504-506. The protein (SEQ ID NO:12) encoded by Clone 17132296.0.4 was predicted by the PSORT computer program to be localized in the microbody (peroxisome) with a certainty of 0.6400. The PSORT and SignalP computer programs predicted that there is no signal peptide. The nucleic acid (SEQ ID NO:11) and amino acid (SEQ ID NO:12) sequences of Clone 17132296.0.4 are shown below in Table 7.

TABLE 7

Clone 17132296.0.4
Translated Protein—Frame: 3-Nucleotide 141 to 503

| | | |
|---|---|---|
| 1 | AGAGATTCATGGCTGGGGAACCCTTGCTGGTGTTCAGAATCTGGA | (SEQ ID NO:11) |
| 46 | TCTACAGTTTCTCCCTTTACGACCCACAGATTTAGGCCCTGATTC | |
| 91 | TCTTCTTTTTCAGGAATGTGCACCTCACCCTGTTCTCCCAGACCT | |
| 136 | TGGGGATGAAGGAAACAGGAGCCTCACCCAGGAGGCTCAAGGCCA | |
| | MetLysGluThrGlyAlaSerProArgArgLeuLysAlaL | (SEQ ID NO:12) |
| 181 | AAACTCTGACCCAAACTACCTCAGGAGCCCCTGGCCCTGGCTTCC | |
| | ysThrLeuThrGlnThrThrSerGlyAlaProGlyProGlyPheP | |
| 226 | CCCCTGCTCCAGAGTTTCTGCCCTGCCCACACACACACACCCTCT | |
| | roProAlaProGluPheLeuProCysProHisThrHisThrLeuP | |
| 271 | TCCACCCTCAGAGGCCCCGGTGTCCTGCCCCACGCTCTACCCCAG | |
| | heHisProGlnArgProArgCysProAlaProArgSerThrProG | |
| 316 | AGCCCCACGGGTGGCTTTATAAAAGTGCCGGGCCCAGCCCTCTAG | |
| | luProHisGlyTrpLeuTyrLysSerAlaGlyProSerProLeuA | |
| 361 | CAGGAGGGGAATGCTGGGCATCTGGGTGTGGGACCCCCGGGGAAC | |
| | laGlyGlyGluCysTrpAlaSerGlyCysGlyThrProGlyGluG | |
| 406 | AGCCTGTGGTCTGGACTCCTGCATCTATGAGGGGACAGACGTGGC | |
| | lnProValValTrpThrProAlaSerMetArgGlyGlnThrTrpL | |
| 451 | TTCCCTTCCGGATGATGGGGTACCCACAGATGATGGAGGCCAGGG | |
| | euProPheArgMetMetGlyTyrProGlnMetMetGluAlaArgV | |
| 496 | TCCCTCAATAAAAGAAGGGGTGCAAAAA | |
| | alProGln | |

Analysis of the sequence databases using the BLAST P and BLASTX computer programs revealed that the protein encoded by Clone 17132296.0.4 has 38 of 105 residues (36%) identical to, and 44 of 105 residues (41%) positive with, the 995 residue human atrophin-related protein ARP (ACC:AAD27584).

The proteins of the invention encoded by Clone 17132296.0.4 include the protein disclosed as being encoded by the ORFs described herein, as well as any mature protein arising therefrom as a result of post-translational modifications. Thus, the proteins of the invention encompass both a precursor and any active forms of the 17132296.0.4 protein.

Experimental results presented in Example 16 demonstrate that Clone 17132296 is over-expressed, relative to normal tissue cell lines, in ovarian cancer, breast cancer, and colon cancer. These results suggest that the nucleic acid or amino acid sequences clone may be useful in the detection, diagnosis, or treatment of these cancers.

### PRO7 and PRO8 Nucleic Acids and Polypeptides

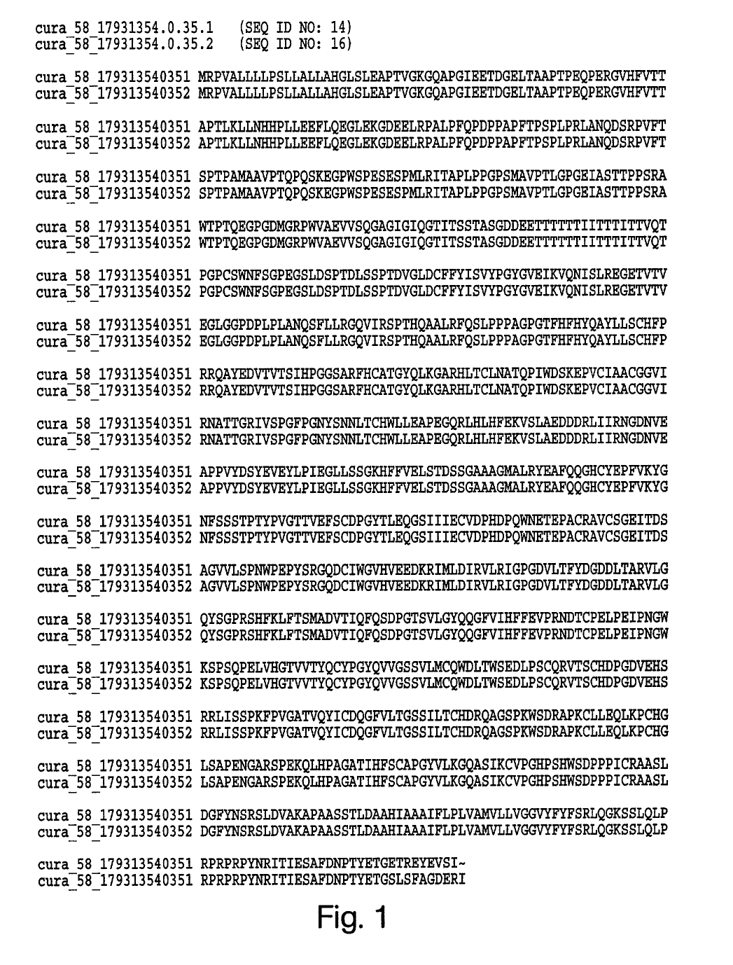

A PRO7 nucleic acid according to the invention includes the nucleic acid sequence represented in Clone 17931354.0.35.1. A PRO8 nucleic acid according to the invention includes the nucleic acid sequence represented in Clone 17931354.0.35.2 (PROX 8). The two clones resemble each other in that they are identical over most of their common sequences (i.e., those nucleic acids encoding amino acid residues 1-984), and differ only at the carboxyl-terminus (see, FIG. 1. In addition, Clone 17931354.0.35.2 extends one amino acid residue further at the carboxyl-terminus than does Clone 17931354.0.35.1.

The nucleic acid sequences represented in Clone 17931354.0.35.1 and Clone 17931354.0.35.2 were observed in the pituitary gland, and were also found to occur in brain, fetal brain, and fetal liver.

A representation of the nucleotide sequence of Clone 17931354.0.35.1 (PROX 7) is represented in Table 8 and includes a nucleotide sequence (SEQ ID NO:13) of 3863 bp. This nucleotide sequence has an open reading frame (ORF) encoding a polypeptide of 993 amino acid residues (SEQ ID NO:14) with a predicted molecular weight of 107523.8 Daltons. The start codon is located at nucleotides178-180 and the stop codon is located at nucleotides 3157-3159. The protein (SEQ ID NO:14) encoded by Clone 17931354.0.35.1 was predicted by the PSORT computer program to be localized to the plasma membrane with a certainty of 0.6760. The PSORT and SignalP computer programs predicted that there is a signal peptide, with the most likely cleavage site located between residues 19 and 20, at the sequence AHG-LS. The nucleic acid (SEQ ID NO:13) and amino acid (SEQ ID NO:14) sequences of Clone 17931354.0.35.1 are shown below in Table 8.

TABLE 8

+HL,1Clone 1793 1354.0.35.1
Translated Protein—Frame: 1-Nucleotide 178 to 3156

| | | |
|---|---|---|
| 1 | CCAGGCGCTGGCCGTGGTGCTGATTCTGTCAGGCGCTGGCGGCGG | (SEQ ID NO:13) |
| 46 | CAGCGGCGGTGACGGCTGCGGCCCCGCTCCCTCTACCCGGCCGGA | |
| 91 | CCCGGCTCTGCCCCCGCGCCCAAGCCCCACCAAGCCCCCCGCCCT | |
| 136 | CCCGCCGCGGTCCCAGCCCAGGGCGCGGCCGCAACCAGCACCATG | |
| | Met | (SEQ ID NO:14) |
| 181 | CGCCCGGTAGCCCTGCTGCTCCTGCCCTCGCTGCTGGCGCTCCTG | |
| | ArgProValAlaLeuLeuLeuLeuProSerLeuLeuAlaLeuLeu | |
| 226 | GCTCACGGACTCTCTTTAGAGGCCCCAACCGTGGGGAAAGGACAA | |
| | AlaHisGlyLeuSerLeuGluAlaProThrValGlyLysGlyGln | |
| 271 | GCCCCAGGCATCGAGGAGACAGATGGCGAGCTGACAGCAGCCCCC | |
| | AlaProGlyIleGluGluThrAspGlyGluLeuThrAlaAlaPro | |
| 316 | ACACCTGAGCAGCCAGAACGAGGCGTCCACTTTGTCACAACAGCC | |
| | ThrProGluGlnProGluArgGlyValHisPheValThrThrAla | |
| 361 | CCCACCTTGAAGCTGCTCAACCACCACCCGCTGCTTGAGGAATTC | |
| | ProThrLeuLysLeuLeuAsnHisHisProLeuLeuGluGluPhe | |
| 406 | CTACAAGAGGGGCTGGAAAAGGGAGATGAGGAGCTGAGGCCAGCA | |
| | LeuGlnGluGlyLeuGluLysGlyAspGluGluLeuArgProAla | |
| 451 | CTGCCCTTCCAGCCTGACCCACCTGCACCCTTCACCCCAAGTCCC | |
| | LeuProPheGlnProAspProProAlaProPheThrProSerPro | |
| 496 | CTTCCCCGCCTGGCCAACCAGGACAGCCGCCCTGTCTTTACCAGC | |
| | LeuProArgLeuAlaAsnGlnAspSerArgProValPheThrSer | |
| 541 | CCCACTCCAGCCATGGCTGCGGTACCCACTCAGCCCCAGTCCAAG | |
| | ProThrProAlaMetAlaAlaValProThrGlnProGlnSerLys | |
| 586 | GAGGGACCCTGGAGTCCGGAGTCAGAGTCCCCTATGCTTCGAATC | |
| | GluGlyProTrpSerProGluSerGluSerProMetLeuArgIle | |
| 631 | ACAGCTCCCCTACCTCCAGGGCCCAGCATGGCAGTGCCCACCCTA | |
| | ThrAlaProLeuProProGlyProSerMetAlaValProThrLeu | |
| 676 | GGCCCAGGGGAGATAGCCAGCACTACACCCCCCAGCAGAGCCTGG | |
| | GlyProGlyGluIleAlaSerThrThrProProSerArgAlaTrp | |
| 721 | ACACCAACCCAAGAGGGTCCTGGAGACATGGGAAGGCCGTGGGTT | |
| | ThrProThrGlnGluGlyProGlyAspMetGlyArgProTrpVal | |
| 766 | GCAGAGGTTGTGTCCCAGGGCGCAGGGATCGGGATCCAGGGGACC | |
| | AlaGluValValSerGlnGlyAlaGlyIleGlyIleGlnGlyThr | |
| 811 | ATCACCTCCTCCACAGCTTCAGGAGATGATGAGGAGACCACCACT | |
| | IleThrSerSerThrAlaSerGlyAspAspGluGluThrThrThr | |
| 856 | ACCACCACCATCATCACCACCACCATCACCACAGTCCAGACACCA | |
| | ThrThrThrIleIleThrThrThrIleThrThrValGlnThrPro | |
| 901 | GGCCCTTGTAGCTCGAATTTCTCAGGCCCAGAGGGCTCTCTGGAC | |
| | GlyProCysSerTrpAsnPheSerGlyProGluGlySerLeuAsp | |
| 946 | TCCCCTACAGACCTCAGCTCCCCCACTGATGTTGGCCTGGACTGC | |
| | SerProThrAspLeuSerSerProThrAspValGlyLeuAspCys | |
| 991 | TTCTTCTACATCTCTGTCTACCCTGGCTATGGCGTGGAAATCAAG | |
| | PhePheTyrIleSerValTyrProGlyTyrGlyValGluIleLys | |
| 1036 | GTCCAGAATATCAGCCTCCGGGAAGGGGAGACAGTGACTGTGGAA | |
| | ValGlnAsnIleSerLeuArgGluGlyGluThrValThrValGlu | |
| 1081 | GGCCTGGGGGGGCCTGACCCACTGCCCCTGGCCAACCAGTCTTTC | |
| | GlyLeuGlyGlyProAspProLeuProLeuAlaAsnGlnSerPhe | |
| 1126 | CTGCTGCGGGGCCAAGTCATCCGCAGCCCCACCCACCAAGCGGCC | |
| | LeuLeuArgGlyGlnValIleArgSerProThrHisGlnAlaAla | |
| 1171 | CTGAGGTTCCAGAGCCTCCCGCCACCGGCTGGCCCTGGCACCTTC | |
| | LeuArgPheGlnSerLeuProProProAlaGlyProGlyThrPhe | |
| 1216 | CATTTCCATTACCAAGCCTATCTCCTGAGCTGCCACTTTCCCCGT | |
| | HisPheHisTyrGlnAlaTyrLeuLeuSerCysHisPheProArg | |
| 1261 | CGTCAAGCTTATGAAGATGTGACTGTCACCAGCATCCACCCAGGA | |
| | ArgGlnAlaTyrGluAspValThrValThrSerIleHisProGly | |
| 1306 | GGTAGTGCCCGCTTCCATTGTGCAACTGGCTACCAGCTGAAGGGC | |
| | GlySerAlaArgPheHisCysAlaThrGlyTyrGlnLeuLysGly | |
| 1351 | GCCAGGCATCTCACCTGTCTCAATGCCACCCAGCCCATCTGGGAT | |
| | AlaArgHisLeuThrCysLeuAsnAlaThrGlnProIleTrpAsp | |
| 1396 | TCAAAGGAGCCCGTATGCATCGCTGCTTGCGGCGGAGTGATCCGC | |
| | SerLysGluProValCysIleAlaAlaCysGlyGlyValIleArg | |
| 1441 | AATGCCACCACCGGCCGCATCGTCTCTCCAGGCTTCCCGGGCAAC | |
| | AsnAlaThrThrGlyArgIleValSerProGlyPheProGlyAsn | |
| 1486 | TACAGCAACAACCTCACCTGTCACTGGCTGCTTGAGGCTCCTGAG | |
| | TyrSerAsnAsnLeuThrCysHisTrpLeuLeuGluAlaProGlu | |
| 1531 | GGCCAGCGGCTACACCTGCACTTTGAGAAGGTTTCCCTGGCAGAG | |
| | GlyGlnArgLeuHisLeuHisPheGluLysValSerLeuAlaGlu | |
| 1576 | GATGATGACAGGCTCATCATTCGCAATGGGGACAACGTGGAGGCC | |
| | AspAspAspArgLeuIleIleArgAsnGlyAspAsnValGluAla | |
| 1621 | CCACCAGTGTATGATTCCTATGAGGTGGAATACCTGCCCATTGAG | |
| | ProProValTyrAspSerTyrGluValGluTyrLeuProIleGlu | |
| 1666 | GGCCTGCTCAGCTCTGGCAAACACTTCTTTGTTGAGCTCAGTACT | |
| | GlyLeuLeuSerSerGlyLysHisPhePheValGluLeuSerThr | |
| 1711 | GACAGCAGCGGGGCAGCTGCAGGCATGGCCCTGCGCTATGAGGCN | |
| | AspSerSerGlyAlaAlaAlaGlyMetAlaLeuArgTyrGluAla | |
| 1756 | TTCCAGCAGGGCCATTGCTATGAGCCCTTTGTCAAATACGGTAAC | |
| | PheGlnGlnGlyHisCysTyrGluProPheValLysTyrGlyAsn | |
| 1801 | TTCAGCAGCAGCACACCCACCTACCCTGTGGGTACCACTGTGGAG | |
| | PheSerSerSerThrProThrTyrProValGlyThrThrValGlu | |
| 1846 | TTTAGCTGCGACCCTGGCTACACCCTGGAGCAGGGCTCCATCATC | |
| | PheSerCysAspProGlyTyrThrLeuGluGlnGlySerIleIle | |
| 1891 | ATCGAGTGTGTTGACCCCCACGACCCCCAGTGGAATGAGACAGAG | |
| | IleGluCysValAspProHisAspProGlnTrpAsnGluThrGlu | |
| 1936 | CCAGCCTGCCGAGCCGTGTGCAGCGGGGAGATCACAGACTCGGCT | |
| | ProAlaCysArgAlaValCysSerGlyGluIleThrAspSerAla | |
| 1981 | GGCGTGGTACTCTCTCCCAACTGGCCAGAGCCCTACAGTCGTGGG | |
| | GlyValValLeuSerProAsnTrpProGluProTyrSerArgGly | |
| 2026 | CAGGATTGTATCTGGGGTGTGCATGTGGAAGAGGACAAGCGCATC | |
| | GlnAspCysIleTrpGlyValHisValGluGluAspLysArgIle | |
| 2071 | ATGCTGGACATCCGAGTGCTGCGCATAGGCCCTGGTGATGTGCTT | |
| | MetLeuAspIleArgValLeuArgIleGlyProGlyAspValLeu | |
| 2116 | ACCTTCTATGATGGGGATGACCTGACGGCCCGGGTTCTGGGCCAG | |
| | ThrPheTyrAspGlyAspAspLeuThrAlaArgValLeuGlyGln | |
| 2161 | TACTCAGGGCCCCGTAGCCACTTCAAGCTCTTTACCTCCATGGCT | |
| | TyrSerGlyProArgSerHisPheLysLeuPheThrSerMetAla | |
| 2206 | GATGTCACCATTCAGTTCCAGTCGGACCCCGGGACCTCAGTGCTG | |
| | AspValThrIleGlnPheGlnSerAspProGlyThrSerValLeu | |
| 2251 | GGCTACCAGCAGGGCTTCGTCATCCACTTCTTTGAGGTGCCCCGC | |
| | GlyTyrGlnGlnGlyPheValIleHisPhePheGluValProArg | |
| 2296 | AATGACACATGTCCGGAGCTGCCTGAGATCCCCAATGGCTGGAAG | |
| | AsnAspThrCysProGluLeuProGluIleProAsnGlyTrpLys | |
| 2341 | AGCCCATCGCAGCCTGAGCTAGTGCACGGCACCGTGGTCACTTAC | |
| | SerProSerGlnProGluLeuValHisGlyThrValValThrTyr | |
| 2386 | CAGTGCTACCCTGGCTACCAGGTAGTGGGATCCAGTGTCCTCATG | |
| | GlnCysTyrProGlyTyrGlnValValGlySerSerValLeuMet | |
| 2431 | TGCCAGTGGGACCTAACTTGGAGTGAGGACCTGCCCTCATGCCAG | |
| | CysGlnTrpAspLeuThrTrpSerGluAspLeuProSerCysGln | |
| 2476 | AGGGTGACTTCCTGCCACGATCCTGGAGATGTGGAGCACAGCCGA | |
| | ArgValThrSerCysHisAspProGlyAspValGluHisSerArg | |
| 2521 | CGCCTCATATCCAGCCCCAAGTTTCCCGTGGGGGCCACCGTGCAA | |
| | ArgLeuIleSerSerProLysPheProValGlyAlaThrValGln | |
| 2566 | TATATCTGTGACCAGGGTTTTGTGCTGACGGGCAGCTCCATCCTC | |
| | TyrIleCysAspGlnGlyPheValLeuThrGlySerSerIleLeu | |
| 2611 | ACCTGCCATGATCGCCAGGCTGGCAGCCCCAAGTGGAGTGACCGG | |
| | ThrCysHisAspArgGlnAlaGlySerProLysTrpSerAspArg | |
| 2656 | GCCCCTAAATGTCTCCTGGAACAGCTCAAGCCATGCCATGGTCTC | |
| | AlaProLysCysLeuLeuGluGlnLeuLysProCysHisGlyLeu | |
| 2701 | AGTGCCCCTGAGAATGGTGCCCGAAGTCCTGAGAAGCAGCTACAC | |
| | SerAlaProGluAsnGlyAlaArgSerProGluLysGlnLeuHis | |
| 2746 | CCAGCAGGGGCCACCATCCACTTCTCGTGTGCCCCTGGCTATGTG | |
| | ProAlaGlyAlaThrIleHisPheSerCysAlaProGlyTyrVal | |
| 2791 | CTGAAGGGCCAGGCCAGCATCAAGTGTGTGCCTGGGCACCCCTCG | |
| | LeuLysGlyGlnAlaSerIleLysCysValProGlyHisProSer | |
| 2836 | CATTGGAGTGACCCCCCACCCATCTGTAGGGCTGCCTCTCTGGAT | |
| | HisTrpSerAspProProProIleCysArgAlaAlaSerLeuAsp | |
| 2881 | GGGTTCTACAACAGTCGCAGCCTGGATGTTGCCAAGGCACCTGCT | |
| | GlyPheTyrAsnSerArgSerLeuAspValAlaLysAlaProAla | |
| 2926 | GCCTCCAGCACCCTGGATGCTGCCCACATTGCAGCTGCCATCTTC | |
| | AlaSerSerThrLeuAspAlaAlaHisIleAlaAlaAlaIlePhe | |
| 2971 | TTGCCACTGGTGGCGATGGTGTTGTTGGTAGGAGGTGTATACTTC | |
| | LeuProLeuValAlaMetValLeuLeuValGlyGlyValTyrPhe | |
| 3016 | TACTTCTCCAGGCTCCAGGGAAAAAGCTCCCTGCAGCTGCCCCGC | |
| | TyrPheSerArgLeuGlnGlyLysSerSerLeuGlnLeuProArg | |
| 3061 | CCCCGCCCCCGCCCCTACAACCGCATTACCATAGAGTCAGCGTTT | |
| | ProArgProArgProTyrAsnArgIleThrIleGluSerAlaPhe | |
| 3106 | GACAATCCAACTTACGAGACTGGAGAGACGAGAGAATATGAAGTC | |
| | AspAsnProThrTyrGluThrGlyGluThrArgGluTyrGluVal | |
| 3151 | TCCATCTAGGTGGGGGCAGTCTAGGGAAGTCAACTCAGACTTGCA | |
| | SerIle | |
| 3196 | CCACAGTCCAGCAGCAAGGCTCCTTGCTTCCTGCTGTCCCTCCAC | |
| 3241 | CTCCTGTATATACCACCTAGGAGGAGATGCCACCAAGCCCTCAAG | |
| 3286 | AAGTTGTGCCCTTCCCCGCCTGCGATGCCCACCATGGCCTATTTT | |
| 3331 | CTTGGTGTCATTGCCCACTTGGGGCCCTTCATTGGGCCCATGTCA | |
| 3376 | GGGGGCATCTACCTGTGGGAAGAACATAGCTGGAGCACAAGCATC | |
| 3421 | AACAGCCAGCATCCTGAGCCTCCTCATGCCCTGGACCAGCCTGGA | |
| 3466 | ACACACTAGCAGAGCAGGAGTACCTTTCTCCACATGACCACCATC | |
| 3511 | CCGCCCTGGCATGGCAACCTGCAGCAGGATTAACTTGACCATGGT | |
| 3556 | GGGAACTGCACCAGGGTACTCCTCACAGCGCCATCACCAATGGCC | |
| 3601 | AAAACTCCTCTCAACGGTGACCTCTGGGTAGTCCTGGCATGCCAA | |
| 3646 | CATCAGCCTCTTGGGAGGTCTCTAGTTCTCTAAAGTTCTGGACAG | |
| 3691 | TTCTGCCTCCTGCCCTGTCCCAGTGGAGGCAGTAATTCTAGGAGA | |
| 3736 | TCCTAAGGGGTTCAGGGGGACCCTACCCCCACCTCAGGTTGGGCT | |
| 3781 | TCCCTGGGCACTCATGCTCCACACCAAAGCAGGACACGCCATTTT | |
| 3826 | CCACTGACCACCCTATACCCTGAGGAAAGGGAGACTTT | |

A representation of the nucleotide sequence of Clone 17931354.0.35.2 (PROX 8) is given in Table 9 and includes a nucleotide sequence (SEQ ID NO:15) of 3879 bp. This nucleotide sequence has an open reading frame (ORF) encoding a polypeptide of 994 amino acid residues (SEQ ID NO:16) with a predicted molecular weight of 107492.8 Daltons. The start codon is located at nucleotides 178-180 and the stop codon is located at nucleotides 3160-3162. The protein (SEQ ID NO:16) encoded by Clone 17931354.0.35.2 was predicted by the PSORT computer program to be localized to the plasma membrane with a certainty of 0.6760. The PSORT and SignalP computer programs predicted that there is a signal peptide, with the most likely cleavage site being located between residues 19 and 20, at the sequence AHG-LS. The nucleic acid (SEQ ID NO:15) and amino acid (SEQ ID NO:16) sequences of Clone 17931354.0.35.2 (PROX 8) are shown below in Table 9.

TABLE 9

Clone 1793 1354.0.35.2
Translated Protein-Frame: 1-Nucleotide 178 to 3159

| | | |
|---|---|---|
| 1 | CCAGGCGCTGGCCGTGGTGCTGATTCTGTCAGGCGCTGGCGGCGG | (SEQ ID NO:15) |
| 46 | CAGCGGCGGTGACGGCTGCGGCCCCGCTCCCTCTACCCGGCCGGA | |
| 91 | CCCGGCTCTGCCCCCGCGCCCAAGCCCCACCAAGCCCCCCGCCCT | |
| 136 | CCCGCCGCGGTCCCAGCCCAGGGCGCGGCCGCAACCAGCACCATG | |
| | Met | (SEQ ID NO:16) |
| 181 | CGCCCGGTAGCCCTGCTGCTCCTGCCCTCGCTGCTGGCGCTCCTG | |
| | ArgProValAlaLeuLeuLeuLeuProSerLeuLeuAlaLeuLeu | |
| 226 | GCTCACGGACTCTCTTTAGAGGCCCCAACCGTGGGGAAAGGACAA | |
| | AlaHisGlyLeuSerLeuGluAlaProThrValGlyLysGlyGln | |
| 271 | GCCCCAGGCATCGAGGAGACAGATGGCGAGCTGACAGCAGCCCCC | |
| | AlaProGlyIleGluGluThrAspGlyGluLeuThrAlaAlaPro | |
| 316 | ACACCTGAGCAGCCAGAACGAGGCGTCCACTTTGTCACAACAGCC | |
| | ThrProGluGlnProGluArgGlyValHisPheValThrThrAla | |
| 361 | CCCACCTTGAAGCTGCTCAACCACCACCCGCTGCTTGAGGAATTC | |
| | ProThrLeuLysLeuLeuAsnHisHisProLeuLeuGluGluPhe | |
| 406 | CTACAAGAGGGGCTGGAAAAGGGAGATGAGGAGCTGAGGCCAGCA | |
| | LeuGlnGluGlyLeuGluLysGlyAspGluGluLeuArgProAla | |
| 451 | CTGCCCTTCCAGCCTGACCCACCTGCACCCTTCACCCCAAGTCCC | |
| | LeuProPheGlnProAspProProAlaProPheThrProSerPro | |
| 496 | CTTCCCCGCCTGGCCAACCAGGACAGCCGCCCTGTCTTTACCAGC | |
| | LeuProArgLeuAlaAsnGlnAspSerArgProValPheThrSer | |
| 541 | CCCACTCCAGCCATGGCTGCGGTACCCACTCAGCCCCAGTCCAAG | |
| | ProThrProAlaMetAlaAlaValProThrGlnProGlnSerLys | |
| 586 | GAGGGACCCTGGAGTCCGGAGTCAGAGTCCCCTATGCTTCGAATC | |
| | GluGlyProTrpSerProGluSerGluSerProMetLeuArgIle | |
| 631 | ACAGCTCCCCTACCTCCAGGGCCCAGCATGGCAGTGCCCACCCTA | |
| | ThrAlaProLeuProProGlyProSerMetAlaValProThrLeu | |
| 676 | GGCCCAGGGGAGATAGCCAGCACTACACCCCCCAGCAGAGCCTGG | |
| | GlyProGlyGluIleAlaSerThrThrProProSerArgAlaTrp | |
| 721 | ACACCAACCCAAGAGGGTCCTGGAGACATGGGAAGGCCGTGGGTT | |
| | ThrProThrGlnGluGlyProGlyAspMetGlyArgProTrpVal | |
| 766 | GCAGAGGTTGTGTCCCAGGGCGCAGGGATCGGGATCCAGGGGACC | |
| | AlaGluValValSerGlnGlyAlaGlyIleGlyIleGlnGlyThr | |
| 811 | ATCACCTCCTCCACAGCTTCAGGAGATGATGAGGAGACCACCACT | |
| | IleThrSerSerThrAlaSerGlyAspAspGluGluThrThrThr | |
| 856 | ACCACCACCATCATCACCACCACCATCACCACAGTCCAGACACCA | |
| | ThrThrThrIleIleThrThrThrIleThrThrValGlnThrPro | |
| 901 | GGCCCTTGTAGCTGGAATTTCTCAGGCCCAGAGGGCTCTCTGGAC | |
| | GlyProCysSerTrpAsnPheSerGlyProGluGlySerLeuAsp | |
| 946 | TCCCCTACAGACCTCAGCTCCCCCACTGATGTTGGCCTGGACTGC | |
| | SerProThrAspLeuSerSerProThrAspValGlyLeuAspCys | |
| 991 | TTCTTCTACATCTCTGTCTACCCTGGCTATGGCGTGGAAATCAAG | |
| | PhePheTyrIleSerValTyrProGlyTyrGlYValGluIleLys | |
| 1036 | GTCCAGAATATCAGCCTCCGGGAAGGGGAGACAGTGACTGTGGAA | |
| | ValGlnAsnIleSerLeuArgGluGlyGluThrValThrValGlu | |
| 1081 | GGCCTGGGGGGGCCTGACCCACTGCCCCTGGCCAACCAGTCTTTC | |
| | GlyLeuGlyGlyProAspProLeuProLeuAlaAsnGlnSerPhe | |
| 1126 | CTGCTGCGGGGCCAAGTCATCCGCAGCCCCACCCACCAAGCGGCC | |
| | LeuLeuArgGlyGlnValIleArgSerProThrHisGlnAlaAla | |
| 1171 | CTGAGGTTCCAGAGCCTCCCGCCACCGGCTGGCCCTGGCACCTTC | |
| | LeuArgPheGlnSerLeuProProProAlaGlyProGlyThrPhe | |
| 1216 | CATTTCCATTACCAAGCCTATCTCCTGAGCTGCCACTTTCCCCGT | |
| | HisPheHisTyrGlnAlaTyrLeuLeuSerCysHisPheProArg | |
| 1261 | CGTCAAGCTTATGAAGATGTGACTGTCACCAGCATCCACCCAGGA | |
| | ArgGlnAlaTyrGluAspValThrValThrSerIleHisProGly | |
| 1306 | GGTAGTGCCCGCTTCCATTGTGCAACTGGCTACCAGCTGAAGGGC | |
| | GlySerAlaArgPheHisCysAlaThrGlyTyrGlnLeuLysGly | |
| 1351 | GCCAGGCATCTCACCTGTCTCAATGCCACCCAGCCCATCTGGGAT | |
| | AlaArgHisLeuThrCysLeuAsnAlaThrGlnProIleTrpAsp | |
| 1396 | TCAAAGGAGCCCGTATGCATCGCTGCTTGCGGCGGAGTGATCCGC | |
| | SerLysGluProValCysIleAlaAlaCysGlyGlyValIleArg | |
| 1441 | AATGCCACCACCGGCCGCATCGTCTCTCCAGGCTTCCCGGGCAAC | |
| | AsnAlaThrThrGlyArgIleValSerProGlyPheProGlyAsn | |
| 1486 | TACAGCAACAACCTCACCTGTCACTGGCTGCTTGAGGCTCCTGAG | |
| | TyrSerAsnAsnLeuThrCysHisTrpLeuLeuGluAlaProGlu | |
| 1531 | GGCCAGCGGCTACACCTGCACTTTGAGAAGGTTTCCCTGGCAGAG | |
| | GlyGlnArgLeuHisLeuHisPheGluLysValSerLeuAlaGlu | |
| 1576 | GATGATGACAGGCTCATCATTCGCAATGGGGACAACGTGGAGGCC | |
| | AspAspAspArgLeuIleIleArgAsnGlyAspAsnValGluAla | |
| 1621 | CCACCAGTGTATGATTCCTATGAGGTGGAATACCTGCCCATTGAG | |
| | ProProValTyrAspSerTyrGluValGluTyrLeuProIleGlu | |
| 1666 | GGCCTGCTCAGCTCTGGCAAACACTTCTTTGTTGAGCTCAGTACT | |
| | GlyLeuLeuSerSerGlyLysHisPhePheValGluLeuSerThr | |
| 1711 | GACAGCAGCGGGGCAGCTGCAGGCATGGCCCTGCGCTATGAGGCN | |
| | AspSerSerGlyAlaAlaAlaGlyMetAlaLeuArgTyrGluAla | |
| 1756 | TTCCAGCAGGGCCATTGCTATGAGCCCTTTGTCAAATACGGTAAC | |
| | PheGlnGlnGlyHisCysTyrGluProPheValLysTyrGlyAsn | |
| 1801 | TTCAGCAGCAGCACACCCACCTACCCTGTGGGTACCACTGTGGAG | |
| | PheSerSerSerThrProThrTyrProValGlyThrThrValGlu | |
| 1846 | TTTAGCTGCGACCCTGGCTACACCCTGGAGCAGGGCTCCATCATC | |
| | PheSerCysAspProGlyTyrThrLeuGluGlnGlySerIleIle | |
| 1891 | ATCGAGTGTGTTGACCCCCACGACCCCCAGTGGAATGAGACAGAG | |
| | IleGluCysValAspProHisAspProGlnTrpAsnGluThrGlu | |
| 1936 | CCAGCCTGCCGAGCCGTGTGCAGCGGGGAGATCACAGACTCGGCT | |
| | ProAlaCysArgAlaValCysSerGlyGluIleThrAspSerAla | |
| 1981 | GGCGTGGTACTCTCTCCCAACTGGCCAGAGCCCTACAGTCGTGGG | |
| | GlyValValLeuSerProAsnTrpProGluProTyrSerArgGly | |
| 2026 | CAGGATTGTATCTGGGGTGTGCATGTGGAAGAGGACAAGCGCATC | |
| | GlnAspCysIleTrpGlyValHisValGluGluAspLysArgIle | |
| 2071 | ATGCTGGACATCCGAGTGCTGCGCATAGGCCCTGGTGATGTGCTT | |
| | MetLeuAspIleArgValLeuArgIleGlyProGlyAspValLeu | |
| 2116 | ACCTTCTATGATGGGGATGACCTGACGGCCCGGGTTCTGGGCCAG | |
| | ThrPheTyrAspGlyAspAspLeuThrAlaArgValLeuGlyGln | |
| 2161 | TACTCAGGGCCCCGTAGCCACTTCAAGCTCTTTACCTCCATGGCT | |
| | TyrSerGlyProArgSerHisPheLysLeuPheThrSerMetAla | |
| 2206 | GATGTCACCATTCAGTTCCAGTCGGACCCCGGGACCTCAGTGCTG | |
| | AspValThrIleGlnPheGlnSerAspProGlyThrSerValLeu | |
| 2251 | GGCTACCAGCAGGGCTTCGTCATCCACTTCTTTGAGGTGCCCCGC | |
| | GlyTyrGlnGlnGlyPheValIleHisPhePheGluValProArg | |
| 2296 | AATGACACATGTCCGGAGCTGCCTGAGATCCCCAATGGCTGGAAG | |
| | AsnAspThrCysProGluLeuProGluIleProAsnGlyTrpLys | |
| 2341 | AGCCCATCGCAGCCTGAGCTAGTGCACGGCACCGTGGTCACTTAC | |
| | SerProSerGlnProGluLeuValHisGlyThrValValThrTyr | |
| 2386 | CAGTGCTACCCTGGCTACCAGGTAGTGGGATCCAGTGTCCTCATG | |
| | GlnCysTyrProGlyTyrGlnValValGlySerSerValLeuMet | |
| 2431 | TGCCAGTGGGACCTAACTTGGAGTGAGGACCTGCCCTCATGCCAG | |
| | CysGlnTrpAspLeuThrTrpSerGluAspLeuProSerCysGln | |
| 2476 | AGGGTGACTTCCTGCCACGATCCTGGAGATGTGGAGCACAGCCGA | |
| | ArgValThrSerCysHisAspProGlyAspValGluHisSerArg | |
| 2521 | CGCCTCATATCCAGCCCCAAGTTTCCCGTGGGGGCCACCGTGCAA | |
| | ArgLeuIleSerSerProLysPheProValGlyAlaThrValGln | |
| 2566 | TATATCTGTGACCAGGGTTTTGTGCTGACGGGCAGCTCCATCCTC | |
| | TyrIleCysAspGlnGlyPheValLeuThrGlySerSerIleLeu | |
| 2611 | ACCTGCCATGATCGCCAGGCTGGCAGCCCCAAGTGGAGTGACCGG | |
| | ThrCysHisAspArgGlnAlaGlySerProLysTrpSerAspArg | |
| 2656 | GCCCCTAAATGTCTCCTGGAACAGCTCAAGCCATGCCATGGTCTC | |
| | AlaProLysCysLeuLeuGluGlnLeuLysProCysHisGlyLeu | |
| 2701 | AGTGCCCCTGAGAATGGTGCCCGAAGTCCTGAGAAGCAGCTACAC | |
| | SerAlaProGluAsnGlyAlaArgSerProGluLysGlnLeuHis | |
| 2746 | CCAGCAGGGGCCACCATCCACTTCTCGTGTGCCCCTGGCTATGTG | |
| | ProAlaGlyAlaThrIleHisPheSerCysAlaProGlyTyrVal | |
| 2791 | CTGAAGGGCCAGGCCAGCATCAAGTGTGTGCCTGGGCACCCCTCG | |
| | LeuLysGlyGlnAlaSerIleLysCysValProGlyHisProSer | |
| 2836 | CATTGGAGTGACCCCCCACCCATCTGTAGGGCTGCCTCTCTGGAT | |
| | HisTrpSerAspProProProIleCysArgAlaAlaSerLeuAsp | |
| 2881 | GGGTTCTACAACAGTCGCAGCCTGGATGTTGCCAAGGCACCTGCT | |
| | GlyPheTyrAsnSerArgSerLeuAspValAlaLysAlaProAla | |
| 2926 | GCCTCCAGCACCCTGGATGCTGCCCACATTGCAGCTGCCATCTTC | |
| | AlaSerSerThrLeuAspAlaAlaHisIleAlaAlaAlaIlePhe | |
| 2971 | TTGCCACTGGTGGCGATGGTGTTGTTGGTAGGAGGTGTATACTTC | |
| | LeuProLeuValAlaMetValLeuLeuValGlyGlyValTyrPhe | |
| 3016 | TACTTCTCCAGGCTCCAGGGAAAAAGCTCCCTGCAGCTGCCCCGC | |
| | TyrPheSerArgLeuGlnGlyLysSerSerLeuGlnLeuProArg | |
| 3061 | CCCCGCCCCCGCCCCTACAACCGCATTACCATAGAGTCAGCGTTT | |
| | ProArgProArgProTyrAsnArgIleThrIleGluSerAlaPhe | |
| 3106 | GACAATCCAACTTACGAGACTGGATCTCTTTCCTTTGCAGGAGAC | |
| | AspAsnProThrTyrGluThrGlySerLeuSerPheAlaGlyAsp | |
| 3151 | GAGAGAATATGAAGTCTCCATCTAGGTGGGGGCAGTCTAGGGAAG | |
| | GluArgIle | |
| 3196 | TCAACTCAGACTTGCACCACAGTCCAGCAGCAAGGCTCCTTGCTT | |
| 3241 | CCTGCTGTCCCTCCACCTCCTGTATATACCACCTAGGAGGAGATG | |
| 3286 | CCACCAAGCCCTCAAGAAGTTGTGCCCTTCCCCGCCTGCGATGCC | |
| 3331 | CACCATGGCCTATTTTCTTGGTGTCATTGCCCACTTGGGGCCCTT | |
| 3376 | CATTGGGCCCATGTCAGGGGGCATCTACCTGTGGGAAGAACATAG | |
| 3421 | CTGGAGCACAAGCATCAACAGCCAGCATCCTGAGCCTCCTCATGC | |
| 3466 | CCTGGACCAGCCTGGAACACACTAGCAGAGCAGGAGTACCTTTCT | |
| 3511 | CCACATGACCACCATCCCGCCCTGGCATGGCAACCTGCAGCAGGA | |
| 3556 | TTAACTTGACCATGGTGGGAACTGCACCAGGGTACTCCTCACAGC | |
| 3601 | GCCATCACCAATGGCCAAAACTCCTCTCAACGGTGACCTCTGGGT | |
| 3646 | AGTCCTGGCATGCCAACATCAGCCTCTTGGGAGGTCTCTAGTTCT | |
| 3691 | CTAAAGTTCTGGACAGTTCTGCCTCCTGCCCTGTCCCAGTGGAGG | |
| 3736 | CAGTAATTCTAGGAGATCCTAAGGGGTTCAGGGGGACCCTACCCC | |
| 3781 | CACCTCAGGTTGGGCTTCCCTGGGCACTCATGCTCCACACCAAAG | |
| 3826 | CAGGACACGCCATTTTCCACTGACCACCCTATACCCTGAGGAAAG | |
| 3871 | GGAGACTTT | |

Analysis of the sequence databases using the BLAST P and BLASTX computer programs revealed that the protein encoded by Clone 17931354.0.35.1 (PROX 7) has 882 of 984 residues (89%) identical to, and 921 of 984 residues (93%) positive with, a 991 residue mouse seizure-related protein 6 precursor (seizure-related protein product 6, type 2) (ACC:Q62223). In addition, the protein encoded by Clone 17931354.0.35.1 was also found to have 391of 785 residues (49%) identical to, and 544 of 785 residues (69%) positive with, the 777 residue fragment of human KIAA0927 protein (ACC:BAA76771).

Analysis of the sequence databases using the BLAST P and BLASTX computer programs revealed that the protein encoded by Clone 17931354.0.35.2 (PROX 8) has 892 of 994 residues (89%) identical to, and 931 of 994 residues (93%) positive with, the mouse seizure-related protein 6 precursor (ACC:Q62223) previously identified for Clone 17931354.0.35.1. In addition, the protein encoded by Clone 17931354.0.35.2 was also found to have 348 of 693 residues (50%) identical to, and 484 of 693 residues (69%) positive with, the 775 residue human DJ268D13.1 (mouse seizure-related gene product 6-like protein) (ACC:CAB46625).

The proteins of the invention encoded by Clone 17931354.0.35.1 and Clone 17931354.0.35.2 include the protein disclosed as being encoded by the ORFs described herein, as well as any mature protein arising therefrom as a result of post-translational modifications. Thus, the proteins of the invention encompass both a precursor and any active forms of the 17931354.0.35.1 and 17931354.0.35.2 proteins.

Experimental results presented in Example 16 show that clone 17931354 is expressed in markedly high levels in two lung cancer cell lines, but not in normal lung cells. These results suggest that the nucleic acid or amino acid sequences clone may be useful in the detection, diagnosis, or treatment of these cancers.

### PRO9, PRO10, PRO11, PRO12, and PRO13 Nucleic Acids and Polypeptides

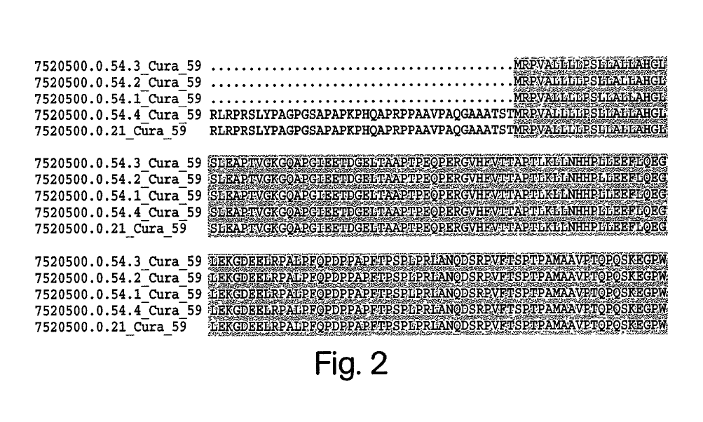

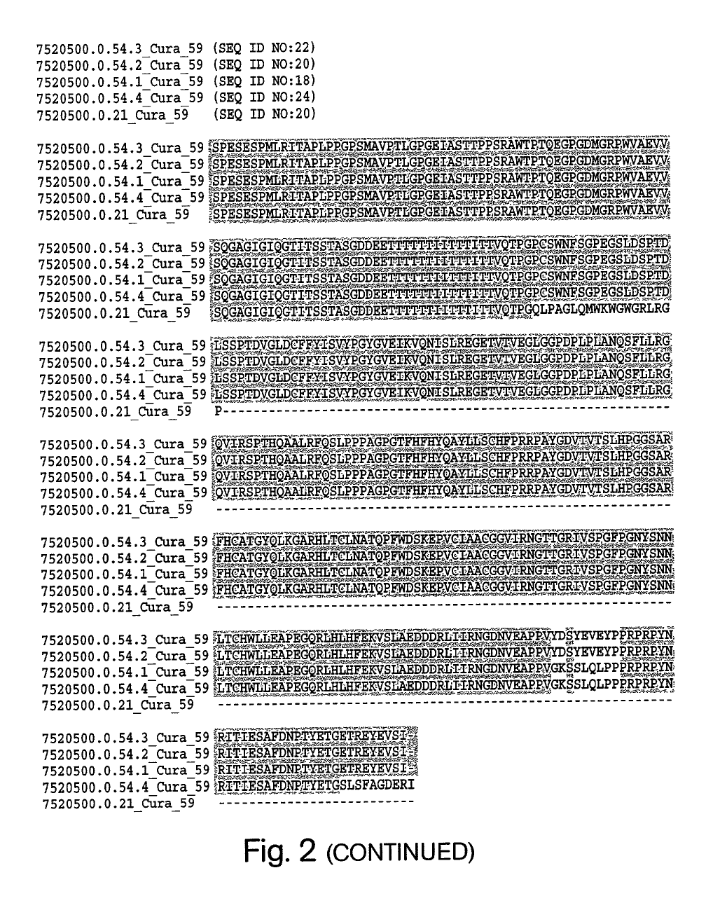

A PRO9, PRO10, PRO11, PRO12, or PRO13 nucleic acid according to the invention includes the nucleic acid sequence represented in Clones 7520500.0.54_{—}1 (PROX 9), 7520500.0.54_{—}2 (PROX 10), 7520500.0.54_{—}3 (PROX 11), 7520500.0.54_{—}4 (PROX 12), and 7520500.0.21 (PROX 13). These clones resemble each other in that they are identical over the majority of their common sequences. For example, Clone 7520500.0.54_{—}2 (PROX 10) and Clone 7520500.0.54_{—}3 (PROX 11) encode identical proteins, although their non-translated regions differ. Similarly, Clone 7520500.0.54_{—}4 (PROX 12) and Clone 7520500.0.21 (PROX 13) encode proteins that possesses extensions with identical sequences in amino-terminal direction, and appear not to be complete, as their amino-terminal amino acid residues are not methionines. In addition, clone 7520500.0.21 (PROX 13) appears to be a 3′ splice variant with respect to the other four clones, as it is terminated far earlier than the others. These and other differences that arise between the clones may be seen by reference to FIG. 2, which gives an alignment of all five proteins encoded by these clones.

The nucleic acid sequences represented in Clone 7520500.0.54_{—}1, Clone 7520500.0.54_{—}2, Clone 7520500.0.54_{—}3, Clone 7520500.0.54_{—}4, and Clone 7520500.0.21 were found in brain, especially fetal brain, and in fetal liver. Representations of the nucleotide sequences of Clone 7520500.0.54_{—}1, Clone 7520500.0.54_{—}2, and Clone 7520500.0.54_{—}3 are presented in Tables 10, 11, and 12, respectively.

Clone 7520500.0.54_{—}1 (PROX 9) includes a nucleotide sequence (SEQ ID NO:17) of 2127 bp. This nucleotide sequence has an open reading frame (ORF) encoding a polypeptide of 525 amino acid residues (SEQ ID NO:18) with a predicted molecular weight of 56284 Daltons. The start codon is located at nucleotides 178-180 and the stop codon is located at nucleotides 1753-1755. The nucleic acid (SEQ ID NO:17) and amino acid (SEQ ID NO:18) sequences of Clone 7520500.0.54_{—}1 (PROX 9) are shown below in Table 10.

TABLE 10
Clone 7520500.0.54.1
Translated Protein - Frame: 1 - Nucleotide 178 to 1752

| | | |
|---|---|---|
| 1 | CCAGGCGCTGGCCGTGGTGCTGATTCTGTCAGGCGCTGGCGGCGG | |
| 46 | CAGCGGCGGTGACGGCTGCGGCCCCGCTCCCTCTACCCGGCCGGA | |
| 91 | CCCGGCTCTGCCCCCGCGCCCAAGCCCCACCAAGCCCCCCGCCCT | |
| 136 | CCCGCCGCGGTCCCAGCCCAGGGCGCGGCCGCAACCAGCACCATG | |
| | Met | |
| 181 | CGCCCGGTAGCCCTGCTGCTCCTGCCCTCGCTGCTGGCGCTCCTG | |
| | ArgProValAlaLeuLeuLeuLeuProSerLeuLeuAlaLeuLeu | |
| 226 | GCTCACGGACTCTCTTTAGAGGCCCCAACCGTGGGGAAAGGACAA | |
| | AlaHisGlyLeuSerLeuGluAlaProThrValGlyLysGlyGln | |
| 271 | GCCCCAGGCATCGAGGAGACAGATGGCGAGCTGACAGCAGCCCCC | |
| | AlaProGlyIleGluGluThrAspGlyGluLeuThrAlaAlaPro | |
| 316 | ACACCTGAGCAGCCAGAACGAGGCGTCCACTTTGTCACAACAGCC | |
| | ThrProGluGlnProGluArgGlyValHisPheValThrThrAla | |
| 361 | CCCACCTTGAAGCTGCTCAACCACCACCCGCTGCTTGAGGAATTC | |
| | ProThrLeuLysLeuLeuAsnHisHisProLeuLeuGluGluPhe | |
| 406 | CTACAAGAGGGGCTGGAAAAGGGAGATGAGGAGCTGAGGCCAGCA | |
| | LeuGlnGluGlyLeuGluLysGlyAspGluGluLeuArgProAla | |
| 451 | CTGCCCTTCCAGCCTGACCCACCTGCACCCTTCACCCCAAGTCCC | |
| | LeuProPheGlnProAspProProAlaProPheThrProSerPro | |
| 496 | CTTCCCCGCCTGGCCAACCAGGACAGCCGCCCTGTCTTTACCAGC | |
| | LeuProArgLeuAlaAsnGlnAspSerArgProValPheThrSer | |
| 541 | CCCACTCCAGCCATGGCTGCGGTACCCACTCAGCCCCAGTCCAAG | |
| | ProThrProAlaMetAlaAlaValProThrGlnProGlnSerLys | |
| 586 | GAGGGACCCTGGAGTCCGGAGTCAGAGTCCCCTATGCTTCGAATC | |
| | GluGlyProTrpSerProGluSerGluSerProMetLeuArgIle | |
| 631 | ACAGCTCCCCTACCTCCAGGGCCCAGCATGGCAGTGCCCACCCTA | |
| | ThrAlaProLeuProProGlyProSerMetAlaValProThrLeu | |
| 676 | GGCCCAGGGGAGATAGCCAGCACTACACCCCCCAGCAGACCCTGG | |
| | GlyProGlyGluIleAlaSerThrThrProProSerArgAlaTrp | |
| 721 | ACACCAACCCAAGAGGGTCCTGGAGACATGGGAAGGCCGTGGGTT | |
| | ThrProThrGlnGluGlyProGlyAspMetGlyArgProTrpVal | |
| 766 | GCAGAGGTTGTGTCCCAGGGCGCAGGGATCGGGATCCAGGGGACC | |
| | AlaGluValValSerGlnGlyAlaGlyIleGlyIleGlnGlyThr | |
| 811 | ATCACCTCCTCCACAGCTTCAGGAGATGATGAGGAGACCACCACT | |
| | IleThrSerSerThrAlaSerGlyAspAspGluGluThrThrThr | |
| 856 | ACCACCACCATCATCACCACCACCATCACCACAGTCCAGACACCA | |
| | ThrThrThrIleIleThrThrThrIleThrThrValGlnThrPro | |
| 901 | GGCCCTTGTAGCTGGAATTTCTCAGGCCCAGAGGGCTCTCTGGAC | |
| | GlyProCysSerTrpAsnPheSerGlyProGluGlySerLeuAsp | |
| 946 | TCCCCTACAGACCTCAGCTCCCCCACTGATGTTGGCCTGGACTGC | |
| | SerProThrAspLeuSerSerProThrAspValGlyLeuAspCys | |
| 991 | TTCTTCTACATCTCTGTCTACCCTGGCTATGGCGTGGAAATCAAG | |
| | PhePheTyrIleSerValTyrProGlyTyrGlyValGluIleLys | |
| 1036 | GTCCAGAATATCAGCCTCCGGGAAGGGGAGACAGTGACTGTGGAA | |
| | ValGlnAsnIleSerLeuArgGluGlyGluThrValThrValGlu | |
| 1081 | GGCCTGGGGGGGCCTGACCCACTGCCCCTGGCCAACCAGTCTTTC | |
| | GlyLeuGlyGlyProAspProLeuProLeuAlaAsnGlnSerPhe | |
| 1126 | CTGCTGCGGGGCCAAGTCATCCGCAGCCCCACCCACCAAGCGGCC | |
| | LeuLeuArgGlyGlnValIleArgSerProThrHisGlnAlaAla | |
| 1171 | CTGAGGTTCCAGAGCCTCCCGCCACCGGCTGGCCCTGGCACCTTC | |
| | LeuArgPheGlnSerLeuProProProAlaGlyProGlyThrPhe | |
| 1216 | CATTTCCATTACCAAGCCTATCTCCTGAGCTGCCACTTTCCCCGT | |
| | HisPheHisTyrGlnAlaTyrLeuLeuSerCysHisPheProArg | |
| 1261 | CGTCCAGCTTATGGAGATGTGACTGTCACCAGCCTCCACCCAGGG | |
| | ArgProAlaTyrGlyAspValThrValThrSerLeuHisProGly | |
| 1306 | GGTAGTGCCCGCTTCCATTGTGCCACTGGCTACCAGCTGAAGGGC | |
| | GlySerAlaArgPheHisCysAlaThrGlyTyrGlnLeuLysGly | |
| 1351 | GCCAGGCATCTCACCTGTCTCAATGCCACCCAGCCCTTCTGGGAT | |
| | AlaArgHisLeuThrCysLeuAsnAlaThrGlnProPheTrpAsp | |
| 1396 | TCAAAGGAGCCCGTCTGCATCGCTGCTTGCGGCGGAGTGATCCGC | |
| | SerLysGluProValCysIleAlaAlaCysGlyGlyValIleArg | |
| 1441 | AATGGCACCACCGGCCGCATCGTCTCTCCAGGCTTCCCGGGCAAC | |
| | AsnGlyThrThrGlyArgIleValSerProGlyPheProGlyAsn | |
| 1486 | TACAGCAACAACCTCACCTGTCACTGGCTGCTTGAGGCTCCTGAG | |
| | TyrSerAsnAsnLeuThrCysHisTrpLeuLeuGluAlaProGlu | |
| 1531 | GGCCAGCGGCTACACCTGCACTTTGAGAAGGTTTCCCTGGCAGAG | |
| | GlyGlnArgLeuHisLeuHisPheGluLysValSerLeuAlaGlu | |
| 1576 | GATGATGACAGGCTCATCATTCGCAATGGGGACAACGTGGAGGCC | |
| | AspAspAspArgLeuIleIleArgAsnGlyAspAsnValGluAla | |
| 1621 | CCACCAGTGGGAAAAAGCTCCCTGCAGCTGCCCCGCCCCCGCCCC | |
| | ProProValGlyLysSerSerLeuGlnLeuProArgProArgPro | |
| 1666 | CGCCCCTACAACCGCATTACCATAGAGTCAGCGTTTGACAATCCA | |
| | ArgProTyrAsnArgIleThrIleGluSerAlaPheAspAsnPro | |
| 1711 | ACTTACGAGACTGGAGAGACGAGAGAATATGAAGTCTCCATCTAG | |
| | ThrTyrGluThrGlyGluThrArgGluTyrGluValSerIle | (SEQ ID NO:18) |
| 1756 | GTGGGGGCAGTCTAGGGAAGTCAACTCAGACTTGCACCACAGTCC | |
| 1801 | AGCAGCAAGGCTCCTTGCTTCCTGCTGTCCCTCCACCTCCTGTAT | |
| 1846 | ATACCACCTAGGAGGAGATGCCACCAAGCCCTCAAGAAGTTGTGC | |
| 1891 | CCTTCCCCGCCTGCGATGCCCACCATGGCCTATTTTCTTGGTGTC | |
| 1936 | ATTGCCCACTTGGGGCCCTTGCATTGGGCCATGTACAGGGGGCAT | |
| 1981 | CTACCTGTGGGGAAGAACATAGCTGGGAGCACAAGCTTCAACAGC | |
| 2026 | CAGCATTCCTTGAGCCTCCTTCATGGCCCTGGGACCAGCCTGGGG | |
| 2071 | AACACANTTAGGCAGGAGCAGGGAGTTACCTTGTTTCACATGACC | |
| 2116 | ACCAACCATTCC | (SEQ ID NO:17) |

Clone 750500.0.54_{—}2 (PROX 10) includes a nucleotide sequence (SEQ ID NO:19) of 2127 bp. This nucleotide sequence has an open reading frame (ORF) encoding a polypeptide of 525 amino acid residues (SEQ ID NO:20) with a predicted molecular weight of 56463 Daltons. The start codon is located at nucleotides 178-180 and the stop codon is located at nucleotides 1753-1755. The nucleic acid (SEQ ID NO:19) and amino acid (SEQ ID NO:20) sequences of Clone 7520500.0.54_{—}2 (PROX 10) are shown below in Table 11.

TABLE 11
Clone 7520500.0.54.2
Translated Protein - Frame: 1 - Nucleotide 178 to 1752

| | | |
|---|---|---|
| 1 | CCAGGCGCTGGCCGTGGTGCTGATTCTGTCAGGCGCTGGCGGCGG | |
| 46 | CAGCGGCGGTGACGGCTGCGGCCCCGCTCCCTCTACCCGGCCGGA | |
| 91 | CCCGGCTCTGCCCCCGCGCCCAAGCCCCACCAAGCCCCCCGCCCT | |
| 136 | CCCGCCGCGGTCCCAGCCCAGGGCGCGGCCGCAACCAGCACATG | |
| | Met | |
| 181 | CGCCCGGTAGCCCTGCTGCTCCTGCCCTCGCTGCTGGCGCTCCTG | |
| | ArgProValAlaLeuLeuLeuLeuProSerLeuLeuAlaLeuLeu | |
| 226 | GCTCACGGACTCTCTTTAGAGGCCCCAACCGTGGGGAAAGGACAA | |
| | AlaHisGlyLeuSerLeuGluAlaProThrValGlyLysGlyGln | |
| 271 | GCCCCAGGCATCGAGGAGACAGATGGCGAGCTGACAGCAGCCCCC | |
| | AlaProGlyIleGluGluThrAspGlyGluLeuThrAlaAlaPro | |
| 316 | ACACCTGAGCAGCCAGAACGAGGCGTCCACTTTGTCACAACAGCC | |
| | ThrProGluGlnProGluArgGlyValHisPheValThrThrAla | |
| 361 | CCCACCTTGAAGCTGCTCAACCACCACCCGCTGCTTGAGGAATTC | |
| | ProThrLeuLysLeuLeuAsnHisHisProLeuLeuGluGluPhe | |
| 406 | CTACAAGAGGGGCTGGAAAAGGGAGATGAGGAGCTGAGGCCAGCA | |
| | LeuGlnGluGlyLeuGluLysGlyAspGluGluLeuArgProAla | |
| 451 | CTGCCCTTCCAGCCTGACCCACCTGCACCCTTCACCCCAAGTCCC | |
| | LeuProPheGlnProAspProProAlaProPheThrProSerPro | |
| 496 | CTTCCCCGCCTGGCCAACCAGGACAGCCGCCCTGTCTTTACCAGC | |
| | LeuProArgLeuAlaAsnGlnAspSerArgProValPheThrSer | |
| 541 | CCCACTCCAGCCATGGCTGCGGTACCCACTCAGCCCCAGTCCAAG | |
| | ProThrProAlaMetAlaAlaValProThrGlnProAlnSerLys | |
| 586 | GAGGGACCCTGGAGTCCGGAGTCAGAGTCCCCTATGCTTCGAATC | |
| | GluGlyProTrpSerProGluSerGluSerProMetLeuArgIle | |
| 631 | ACAGCTCCCCTACCTCCAGGGCCCAGCATGGCAGTGCCCACCCTA | |
| | ThrAlaProLeuProProGlyProSerMetAlaValProThrLeu | |
| 676 | GGCCCAGGGGAGATAGCCAGCACTACACCCCCCAGCAGAGCCTGG | |
| | GlyProGlyGluIleAlaSerThrThrProProSerArgAlaTrp | |
| 721 | ACACCAACCCAAGAGGGTCCTGGAGACATGGGAAGGCCGTGGGTT | |
| | ThrProThrGlnGluGlyProGlyAspMetGlyArgProTrpVal | |
| 766 | GCAGAGGTTGTGTCCCAGGGCGCAGGGATCGGGATCCAGGGGACC | |
| | AlaGluValValSerGlnGlyAlaGlyIleGlyIleGlnGlyThr | |
| 811 | ATCACCTCCTCCACAGCTTCAGGAGATGATGAGGAGACCACCACT | |
| | IleThrSerSerThrAlaSerGlyAspAspGluGluThrThrThr | |
| 856 | ACCACCACCATCATCACCACCACCATCACCACAGTCCAGACACCA | |
| | ThrThrThrIleIleThrThrThrIleThrThrValGlnThrPro | |
| 901 | GGCCCTTGTAGCTGGAATTTCTCAGGCCCAGAGGGCTCTCTGGAC | |
| | GlyProCysSerTrpAsnPheSerGlyProGluGlySerLeuAsp | |
| 946 | TCCCCTACAGACCTCAGCTCCCCCACTGATGTTGGCCTGGACTGC | |
| | SerProThrAspLeuSerSerProThrAspValGlyLeuAspCys | |
| 991 | TTCTTCTACATCTCTGTCTACCCTGGCTATGGCGTGGAAATCAAG | |
| | PhePheTyrIleSerValTyrProGlyTyrGlyValGluIleLys | |
| 1036 | GTCCAGAATATCAGCCTCCGGGAAGGGGAGACAGTGACTGTGGAA | |
| | ValGlnAsnIleSerLeuArgGluGlyGluThrValThrValGlu | |
| 1081 | GGCCTGGGGGGGCCTGACCCACTGCCCCTGGCCAACCAGTCTTTC | |
| | GlyLeuGlyGlyProAspProLeuProLeuAlaAsnGlnSerPhe | |
| 1126 | CTGCTGCGGGGCCAAGTCATCCGCAGCCCCACCCACCAAGCGGCC | |
| | LeuLeuArgGlyGlnValIleArgSerProThrHisGlnAlaAla | |
| 1171 | CTGAGGTTCCAGAGCCTCCCGCCACCGGCTGGCCCTGGCACCTTC | |
| | LeuArgPheGlnSerLeuProProProAlaGlyProGlyThrPhe | |
| 1216 | CATTTCCATTACCAAGCCTATCTCCTGAGCTGCCACTTTCCCCGT | |
| | HisPheHisTyrGlnAlaTyrLeuLeuSerCysHisPheProArg | |
| 1261 | CGTCCAGCTTATGGAGATGTGACTGTCACCAGCCTCCACCCAGGG | |
| | ArgProAlaTyrGlyAspValThrValThrSerLeuHisProGly | |
| 1306 | GGTAGTGCCCGCTTCCATTGTGCCACTGGCTACCAGCTGAAGGGC | |
| | GlySerAlaArgPheHisCysAlaThrGlyTyrGlnLeuLysGly | |
| 1351 | GCCAGGCATCTCACCTGTCTCAATGCCACCCAGCCCTTCTGGGAT | |
| | AlaArgHisLeuThrCysLeuAsnAlaThrGlnProPheTrpAsp | |
| 1396 | TCAAAGGAGCCCGTCTGCATCGCTGCTTGCGGCGGAGTGATCCGC | |
| | SerLysGluProValCysIleAlaAlaCysGlyGlyValIleArg | |
| 1441 | AATGGCACCACCGGCCGCATCGTCTCTCCAGGCTTCCCGGGCAAC | |
| | AsnGlyThrThrGlyArgIleValSerProGlyPheProGlyAsn | |
| 1486 | TACAGCAACAACCTCACCTGTCACTGGCTGCTTGAGGCTCCTGAG | |
| | TyrSerAsnAsnLeuThrCysHisTrpLeuLeuGluAlaProGlu | |
| 1531 | GGCCAGCGGCTACACCTGCACTTTGAGAAGGTTTCCCTGGCAGAG | |
| | GlyGlnArgLeuHisLeuHisPheGluLysValSerLeuAlaGlu | |
| 1576 | GATGATGACAGGCTCATCATTCGCAATGGGGACAACGTGGAGGCC | |
| | AspAspAspArgLeuIleIleArgAsnGlyAspAsnValGluAla | |
| 1621 | CCACCAGTGTATGATTCCTATGAGGTGGAATACCCGCCCCGCCCC | |
| | ProProValTyrAspSerTyrGluValGluTyrProProArgPro | |
| 1666 | CGCCCCTACAACCGCATTACCATAGAGTCAGCGTTTGACAATCCA | |
| | ArgProTyrAsnArgIleThrIleGluSerAlaPheAspAsnPro | |
| 1711 | ACTTACGAGACTGGAGAGACGAGAGAATATGAAGTCTCCATCTAG | |
| | ThrTyrGluThrGlyGluThrArgGluTyrGluValSerIle | (SEQ ID NO:20) |
| 1756 | GTGGGGGCAGTCTAGGGAAGTCAACTCAGACTTGCACCACAGTCC | |
| 1801 | AGCAGCAAGGCTCCTTGCTTCCTGCTGTCCCTCCACCTCCTGTAT | |
| 1846 | ATACCACCTAGGAGGAGATGCCACCAAGCCCTCAAGAAGTTGTGC | |
| 1891 | CCTTCCCCGCCTGCGATGCCCACCATGGCCTATTTTCTTGGTGTC | |
| 1936 | ATTGCCCACTTGGGGCCCTTGCATTGGGCCATGTACAGGGGGCAT | |
| 1981 | CTACCTGTGGGGAAGAACATAGCTGGGAGCACAAGCTTCAACAGC | |
| 2026 | CAGCATTCCTTGAGCCTCCTTCATGGCCCTGGGACCAGCCTGGGG | |
| 2071 | AACACANTTAGGCAGGAGCAGGGAGTTACCTTGTTTCACATGACC | |
| 2116 | ACCAACCATTCC | (SEQ ID NO:19) |

Clone 7520500.0.54_{—}3 (PROX 11) includes a nucleotide sequence (SEQ ID NO:21) of 1988 bp. This nucleotide sequence has an open reading frame (ORF) encoding a polypeptide of 525 amino acid residues (SEQ ID NO:22) with a predicted molecular weight of 56463 Daltons. The polypeptide (SEQ ID NO:22) encoded by the nucleic acid sequence is the same as that of the polypeptide (SEQ ID NO:20) encoded by clone 7520500.0.54_{—}2 (PROX 10). The start codon is located at nucleotides 178-180 and the stop codon is located at nucleotides 1753-1755. The nucleic acid (SEQ ID NO:21) and amino acid (SEQ ID NO:22) sequences of Clone 7520500.0.54_{—}3 (PROX 11) are shown below in Table 12.

TABLE 12
Clone 7520500.0.54.3
Translated Protein - Frame: 1 - Nucleotide 178 to 1752

| | | |
|---|---|---|
| 1 | CCAGGCGCTGGCCGTGGTGCTGATTCTGTCAGGCGCTGGCGGCGG | |
| 46 | CAGCGGCGGTGACGGCTGCGGCCCCGCTCCCTCTACCCGGCCGGA | |
| 91 | CCCGGCTCTGCCCCCGCGCCCAAGCCCCACCAAGCCCCCCGCCCT | |
| 136 | CCCGCCGCGGTCCCAGCCCAGGGCGCGGCCGCAACCAGCACCATG | |
| | Met | |
| 181 | CGCCCGGTAGCCCTGCTGCTCCTGCCCTCGCTGCTGGCGCTCCTG | |
| | ArgProValAlaLeuLeuLeuLeuProSerLeuLeuAlaLeuLeu | |
| 226 | GCTCACGGACTCTCTTTAGAGGCCCCAACCGTGGGGAAAGGACAA | |
| | AlaHisGlyLeuSerLeuGluAlaProThrValGlyLysGlyGln | |
| 271 | GCCCCAGGCATCGAGGAGACAGATGGCGAGCTGACAGCAGCCCCC | |
| | AlaProGlyIleGluGluThrAspGlyGluLeuThrAlaAlaPro | |
| 316 | ACACCTGAGCAGCCAGAACGAGGCGTCCACTTTGTCACAACAGCC | |
| | ThrProGluGlnProGluArgGlyValHisPheValThrThrAla | |
| 361 | CCCACCTTGAAGCTGCTCAACCACCACCCGCTGCTTGAGGAATTC | |
| | ProThrLeuLysLeuLeuAsnHisHisProLeuLeuGluGluPhe | |
| 406 | CTACAAGAGGGGCTGGAAAAGGGAGATGAGGAGCTGAGGCCAGCA | |
| | LeuGlnGluGlyLeuGluLysGlyAspGluGluLeuArgProAla | |
| 451 | CTGCCCTTCCAGCCTGACCCACCTGCACCCTTCACCCCAAGTCCC | |
| | LeuProPheGlnProAspProProAlaProPheThrProSerPro | |
| 496 | CTTCCCCGCCTGGCCAACCAGGACAGCCGCCCTGTCTTTACCAGC | |
| | LeuProArgLeuAlaAsnGlnAspSerArgProValPheThrSer | |
| 541 | CCCACTCCAGCCATGGCTGCGGTACCCACTCAGCCCCAGTCCAAG | |
| | ProThrProAlaMetAlaAlaValProThrGlnProGlnSerLys | |
| 586 | GAGGGACCCTGGAGTCCGGAGTCAGAGTCCCCTATGCTTCGAATC | |
| | GluGlyProTrpSerProGluSerGluSerProMetLeuArgIle | |
| 631 | ACAGCTCCCCTACCTCCAGGGCCCAGCATGGCAGTGCCCACCCTA | |
| | ThrAlaProLeuProProGlyProSerMetAlaValProThrLeu | |
| 676 | GGCCCAGGGGAGATAGCCAGCACTACACCCCCCAGCAGAGCCTGG | |
| | GlyProGlyGluIleAlaSerThrThrProProSerArgAlaTrp | |
| 721 | ACACCAACCCAAGAGGGTCCTGGAGACATGGGAAGGCCGTGGGTT | |
| | ThrProThrGlnGluGlyProGlyAspMetGlyArgProTrpVal | |
| 766 | GCAGAGGTTGTGTCCCAGGGCGCAGGGATCGGGATCCAGGGGACC | |
| | AlaGluValValSerGlnGlyAlaGlyIleGlyIleGlnGlyThr | |
| 811 | ATCACCTCCTCCACAGCTTCAGGAGATGATGAGGAGACCACCACT | |
| | IleThrSerSerThrAlaSerGlyAspAspGluGluThrThrThr | |
| 856 | ACCACCACCATCATCACCACCACCATCACCACAGTCCAGACACCA | |
| | ThrThrThrIleIleThrThrThrIleThrThrValGlnThrPro | |
| 901 | GGCCCTTGTAGCTGGAATTTCTCAGGCCCAGAGGGCTCTCTGGAC | |
| | GlyProCysSerTrpAsnPheSerGlyProGluGlySerLeuAsp | |
| 946 | TCCCCTACAGACCTCAGCTCCCCCACTGATGTTGGCCTGGACTGC | |
| | SerProThrAspLeuSerSerProThrAspValGlyLeuAspCys | |
| 991 | TTCTTCTACATCTCTGTCTACCCTGGCTATGGCGTGGAAATCAAG | |
| | PhePheTyrIleSerValTyrProGlyTyrGlyValGluIleLys | |
| 1036 | GTCCAGAATATCAGCCTCCGGGAAGGGGAGACAGTGACTGTGGAA | |
| | ValGlnAsnIleSerLeuArgGluGlyGluThrValThrValGlu | |
| 1081 | GGCCTGGGGGGGCCTGACCCACTGCCCCTGGCCAACCAGTCTTTC | |
| | GlyLeuGlyGlyProAspProLeuProLeuAlaAsnGlnSerPhe | |
| 1126 | CTGCTGCGGGGCCAAGTCATCCGCAGCCCCACCCACCAAGCGGCC | |
| | LeuLeuArgGlyGlnValIleArgSerProThrHisGlnAlaAla | |
| 1171 | CTGAGGTTCCAGAGCCTCCCGCCACCGGCTGGCCCTGGCACCTTC | |
| | LeuArgPheGlnSerLeuProProProAlaGlyProGlyThrPhe | |
| 1216 | CATTTCCATTACCAAGCCTATCTCCTGAGCTGCCACTTTCCCCGT | |
| | HisPheHisTyrGlnAlaTyrLeuLeuSerCysHisPheProArg | |
| 1261 | CGTCCAGCTTATGGAGATGTGACTGTCACCAGCCTCCACCCAGGG | |
| | ArgProAlaTyrGlyAspValThrValThrSerLeuHisProGly | |
| 1306 | GGTAGTGCCCGCTTCCATTGTGCCACTGGCTACCAGCTGAAGGGC | |
| | GlySerAlaArgPheHisCysAlaThrGlyTyrGlnLeuLysGly | |
| 1351 | GCCAGGCATCTCACCTGTCTCAATGCCACCCAGCCCTTCTGGGAT | |
| | AlaArgHisLeuThrCysLeuAsnAlaThrGlnProPheTrpAsp | |
| 1396 | TCAAAGGAGCCCGTCTGCATCGCTGCTTGCGGCGGAGTGATCCGC | |
| | SerLysGluProValCysIleAlaAlaCysGlyGlyValIleArg | |
| 1441 | AATGGCACCACCGGCCGCATCGTCTCTCCAGGCTTCCCGGGCAAC | |
| | AsnGlyThrThrGlyArgIleValSerProGlyPheProGlyAsn | |
| 1486 | TACAGCAACAACCTCACCTGTCACTGGCTGCTTGAGGCTCCTGAG | |
| | TyrSerAsnAsnLeuThrCysHisTrpLeuLeuGluAlaProGlu | |
| 1531 | GGCCAGCGGCTACACCTGCACTTTGAGAAGGTTTCCCTGGCAGAG | |
| | GlyGlnArgLeuHisLeuHisPheGluLysValSerLeuAlaGlu | |
| 1576 | GATGATGACAGGCTCATCATTCGCAATGGGGACAACGTGGAGGCC | |
| | AspAspAspArgLeuIleIleArgAsnGlyAspAsnValGluAla | |
| 1621 | CCACCAGTGTATGATTCCTATGAGGTGGAATACCCGCCCCGCCCC | |
| | ProProValTyrAspSerTyrGluValGluTyrProProArgPro | |
| 1666 | CGCCCCTACAACCGCATTACCATAGAGTCAGCGTTTGACAATCCA | |
| | ArgProTyrAsnArgIleThrIleGluSerAlaPheAspAsnPro | |
| 1711 | ACTTACGAGACTGGAGAGACGAGAGAATATGAAGTCTCCATCTAG | |
| | ThrTyrGluThrGlyGluThrArgGluTyrGluValSerIle | (SEQ ID NO:22) |
| 1756 | GTGGGGGCAGTCTAGGGAAGTCAACTCAGACTTGCACCACAGTCC | |
| 1801 | AGCAGCAAGGCTCCTTGCTTCCTGCTGTCCCTCCACCTCCTGTAT | |
| 1846 | ATACCACCTAGGAGGAGATGCCACCAAGCCACTTTGTACATGTAA | |
| 1891 | TGTATTATATGGGGTCTGGGCTCCAGCCAGAGAACAATCTTTTAT | |
| 1936 | TTCTGTTGTTTCCTTATTAAAATGGTGTTTTTGGAAAAAAAAAAA | |
| 1981 | AAAAAAAA | (SEQ ID NO:21) |

The proteins of SEQ ID NO:18, SEQ ID NO:20, and SEQ ID NO:22 (i.e., the proteins encoded by Clone 7520500.0.54_{—}1 (PROX 9); Clone 7520500.0.54_{—}2 (PROX 10) and Clone 7520500.0.54_{—}3(PROX 11) were predicted by the PSORT computer program to be localized extracellularly with certainty of 0.8200. The PSORT and SignalP computer programs also predicted that there is a cleavable signal peptide, with the most likely cleavage site located between residues 19 and 20, at the sequence AHG-LS.

Analysis of the protein sequence databases using the BLASTP and BLASTX computer programs revealed that the proteins encoded by Clone 7520500.0.54_{—}1 (PROX 9), Clone 7520500.0.54_{—}2 (PROX 10), and 7520500.0.54_{—}3 (PROX 11) have 421 of 494 residues (85%) identical to, and 448 of 494 residues (90%) positive with, the 977 residue mouse seizure-related protein 6 precursor (ACC:Q62269). In addition, the protein encoded by Clone 7520500.0.54_{—}1 (PROX 9) has 133 of 268 residues (49%) identical to, and 187 of 268 residues (69%) positive with; and the proteins encoded by Clone 7520500.0.54_{—}2 (PROX 10) and Clone 7520500.0.54_{—}3 (PROX 11) have 138 of 286 residues (48%) identical to, and 196 of 286 residues (68%) positive with, a 777 fragment from the human KIAA0927 protein (ACC:BAA76771).

Representations of the nucleotide sequences of Clone 7520500.0.54_{—}4 (PROX 12) and Clone 7520500.0.21 (PROX 13) are presented in Tables 13 and 14, respectively. Clone 7520500.0.54_{—}4 (PROX 12) includes a nucleotide sequence (SEQ ID NO:23) of 2143 bp. This nucleotide sequence has an open reading frame (ORF) encoding a polypeptide of 525 amino acid residues (SEQ ID NO:24) with a predicted molecular weight of 56253 Daltons. the start codon is located at nucleotides 178-180 and the stop codon is located at nucleotides 1756-1758. The protein (SEQ ID NO:24) encoded by Clone 7520500.0.54_{—}4 was predicted by the PSORT computer program to be localized extracellularly with a certainty of 0.8200. The PSORT and SignalP computer programs also predicted that there is a cleavable signal peptide, with the most likely cleavage site located between residues 19 and 20, at sequences AHG-LS. The nucleic acid (SEQ ID NO:23) and amino acid (SEQ ID NO:24) sequences of Clone 7520500.0.54_{—}4 (PROX 12) are shown below in Table 13.

TABLE 13
Clone 7520500.0.54.4
Translated Protein - Frame: 1 - Nucleotide 178 to 1755

| | | |
|---|---|---|
| 1 | CCAGGCGCTGGCCGTGGTGCTGATTCTGTCAGGCGCTGGCGGCGG | |
| 46 | CAGCGGCGGTGACGGCTGCGGCCCCGCTCCCTCTACCCGGCCGGA | |
| 91 | CCCGGCTCTGCCCCCGCGCCCAAGCCCCACCAAGCCCCCCGCCCT | |
| 136 | CCCGCCGCGGTCCCAGCCCAGGGCGCGGCCGCAACCAGCACCATG | |
| | Met | |
| 181 | CGCCCGGTAGCCCTGCTGCTCCTGCCCTCGCTGCTGGCGCTCCTG | |
| | ArgProValAlaLeuLeuLeuLeuProSerLeuLeuAlaLeuLeu | |
| 226 | GCTCACGGACTCTCTTTAGAGGCCCCAACCGTGGGGAAAGGACAA | |
| | AlaHisGlyLeuSerLeuGluAlaProThrValGlyLysGlyGln | |
| 271 | GCCCCAGGCATCGAGGAGACAGATGGCGAGCTGACAGCAGCCCCC | |
| | AlaProGlyIleGluGluThrAspGlyGluLeuThrAlaAlaPro | |
| 316 | ACACCTGAGCAGCCAGAACGAGGCGTCCACTTTGTCACAACAGCC | |
| | ThrProGluGlnProGluArgGlyValHisPheValThrThrAla | |
| 361 | CCCACCTTGAAGCTGCTCAACCACCACCCGCTGCTTGAGGAATTC | |
| | ProThrLeuLysLeuLeuAsnHisHisProLeuLeuGluGluPhe | |
| 406 | CTACAAGAGGGGCTGGAAAAGGGAGATGAGGAGCTGAGGCCAGCA | |
| | LeuGlnGluGlyLeuGluLysGlyAspGluGluLeuArgProAla | |
| 451 | CTGCCCTTCCAGCCTGACCCACCTGCACCCTTCACCCCAAGTCCC | |
| | LeuProPheGlnProAspProProAlaProPheThrProSerPro | |
| 496 | CTTCCCCGCCTGGCCAACCAGGACAGCCGCCCTGTCTTTACCAGC | |
| | LeuProArgLeuAlaAsnGlnAspSerArgProValPheThrSer | |
| 541 | CCCACTCCAGCCATGGCTGCGGTACCCACTCAGCCCCAGTCCAAG | |
| | ProThrProAlaMetAlaAlaValProThrGlnProGlnSerLys | |
| 586 | GAGGGACCCTGGAGTCCGGAGTCAGAGTCCCCTATGCTTCGAATC | |
| | GluGlyProTrpSerProGluSerGluSerProMetLeuArgIle | |
| 631 | ACAGCTCCCCTACCTCCAGGGCCCAGCATGGCAGTGCCCACCCTA | |
| | ThrAlaProLeuProProGlyProSerMetAlaValProThrLeu | |
| 676 | GGCCCAGGGGAGATAGCCAGCACTACACCCCCCAGCAGAGCCTGG | |
| | GlyProGlyGluIleAlaSerThrThrProProSerArgAlaTrp | |
| 721 | ACACCAACCCAAGAGGGTCCTGGAGACATGGGAAGGCCGTGGGTT | |
| | ThrProThrGlnGluGlyProGlyAspMetGlyArgProTrpVal | |
| 766 | GCAGAGGTTGTGTCCCAGGGCGCAGGGATCGGGATCCAGGGGACC | |
| | AlaGluValValSerGlnGlyAlaGlyIleGlyIleGlnGlyThr | |
| 811 | ATCACCTCCTCCACAGCTTCAGGAGATGATGAGGAGACCACCACT | |
| | IleThrSerSerThrAlaSerGlyAspAspGluGluThrThrThr | |
| 856 | ACCACCACCATCATCACCACCACCATCACCACAGTCCAGACACCA | |
| | ThrThrThrIleIleThrThrThrIleThrThrValGlnThrPro | |
| 901 | GGCCCTTGTAGCTGGAATTTCTCAGGCCCAGAGGGCTCTCTGGAC | |
| | GlyProCysSerTrpAsnPheSerGlyProGluGlySerLeuAsp | |
| 946 | TCCCCTACAGACCTCAGCTCCCCCACTGATGTTGGCCTGGACTGC | |
| | SerProThrAspLeuSerSerProThrAspValGlyLeuAspCys | |
| 991 | TTCTTCTACATCTCTGTCTACCCTGGCTATGGCGTGGAAATCAAG | |
| | PhePheTyrIleSerValTyrProGlyTyrGlyValGluIleLys | |
| 1036 | GTCCAGAATATCAGCCTCCGGGAAGGGGAGACAGTGACTGTGGAA | |
| | ValGlnAsnIleSerLeuArgGluGlyGluThrValThrValGlu | |
| 1081 | GGCCTGGGGGGGCCTGACCCACTGCCCCTGGCCAACCAGTCTTTC | |
| | GlyLeuGlyGlyProAspProLeuProLeuAlaAsnGlnSerPhe | |
| 1126 | CTGCTGCGGGGCCAAGTCATCCGCAGCCCCACCCACCAAGCGGCC | |
| | LeuLeuArgGlyGlnValIleArgSerProThrHisGlnAlaAla | |
| 1171 | CTGAGGTTCCAGAGCCTCCCGCCACCGGCTGGCCCTGGCACCTTC | |
| | LeuArgPheGlnSerLeuProProProAlaGlyProGlyThrPhe | |
| 1216 | CATTTCCATTACCAAGCCTATCTCCTGAGCTGCCACTTTCCCCGT | |
| | HisPheHisTyrGlnAlaTyrLeuLeuSerCysHisPheProArg | |
| 1261 | CGTCCAGCTTATGGAGATGTGACTGTCACCAGCCTCCACCCAGGG | |
| | ArgProAlaTyrGlyAspValThrValThrSerLeuHisProGly | |
| 1306 | GGTAGTGCCCGCTTCCATTGTGCCACTGGCTACCAGCTGAAGGGC | |
| | GlySerAlaArgPheHisCysAlaThrGlyTyrGlnLeuLysGly | |
| 1351 | GCCAGGCATCTCACCTGTCTCAATGCCACCCAGCCCTTCTGGGAT | |
| | AlaArgHisLeuThrCysLeuAsnAlaThrGlnProPheTrpAsp | |
| 1396 | TCAAAGGAGCCCGTCTGCATCGCTGCTTGCGGCGGAGTGATCCGC | |
| | SerLysGluProValCysIleAlaAlaCysGlyGlyValIleArg | |
| 1441 | AATGGCACCACCGGCCGCATCGTCTCTCCAGGCTTCCCGGGCAAC | |
| | AsnGlyThrThrGlyArgIleValSerProGlyPheProGlyAsn | |
| 1486 | TACAGCAACAACCTCACCTGTCACTGGCTGCTTGAGGCTCCTGAG | |
| | TyrSerAsnAsnLeuThrCysHisTrpLeuLeuGluAlaProGlu | |
| 1531 | GGCCAGCGGCTACACCTGCACTTTGAGAAGGTTTCCCTGGCAGAG | |
| | GlyGlnArgLeuHisLeuHisPheGluLysValSerLeuAlaGlu | |
| 1576 | GATGATGACAGGCTCATCATTCGCAATGGGGACAACGTGGAGGCC | |
| | AspAspAspArgLeuIleIleArgAsnGlyAspAsnValGluAla | |
| 1621 | CCACCAGTGGGAAAAAGCTCCCTGCAGCTGCCCCGCCCCCGCCCC | |
| | ProProValGlyLysSerSerLeuGlnLeuProArgProArgPro | |
| 1666 | CGCCCCTACAACCGCATTACCATAGAGTCAGCGTTTGACAATCCA | |
| | ArgProTyrAsnArgIleThrIleGluSerAlaPheAspAsnPro | |
| 1711 | ACTTACGAGACTGGATCTCTTTCCTTTGCAGGAGACGAGAGAATA | |
| | ThrTyrGluThrGlySerLeuSerPheAlaGlyAspGluArgIle | (SEQ ID NO:24) |
| 1756 | TGAAGTCTCCATCTAGGTGGGGGCAGTCTAGGGAAGTCAACTCAG | |
| 1846 | CCTCCACCTCCTGTATATACCACCTAGGAGGAGATGCCACCAAGC | |
| 1891 | CCTCAAGAAGTTGTGCCCTTCCCCGCCTGCGATGCCCACCATGGC | |
| 1936 | CTATTTTCTTGGTGTCATTGCCCACTTGGGGCCCTTGCATTGGGC | |
| 1981 | CATGTACAGGGGGCATCTACCTGTGGGGAAGAACATAGCTGGGAG | |
| 2026 | CACAAGCTTCAACAGCCAGCATTCCTTGAGCCTCCTTCATGGCCC | |
| 2071 | TGGGACCAGCCTGGGGAACACANTTAGGCAGGAGCAGGGAGTTAC | |
| 2116 | CTTGTTTCACATGACCACCAACCATTCC | (SEQ ID NO:23) |

Clone 7520500.0.21 (PROX 13) includes a nucleotide sequence (SEQ ID NO:25) of 1482 bp. This nucleotide sequence has an open reading frame (ORF) encoding a polypeptide of 261 amino acid residues (SEQ ID NO:26) with a predicted molecular weight of 56253 Daltons. The start codon is located at nucleotides 178-180 and the stop codon is located at nucleotides 961-963. The protein SEQ ID NO:26) encoded by Clone 7520500.0.21 was predicted by the PSORT computer program to be localized extracellularly with a certainty of 0.8200. The nucleic acid (SEQ ID NO:25) and amino acid (SEQ ID NO:26) sequences of Clone 7520500.0.21 (PROX 13) are shown below in Table 14.

TABLE 14
Clone 7520500.0.21
Translated Protein - Frame: 1 - Nucleotide 178 to 960

| | | |
|---|---|---|
| 1 | CCAGGCGCTGGCCGTGGTGCTGATTCTGTCAGGCGCTGGCGGCGG | |
| 46 | CAGCGGCGGTGACGGCTGCGGCCCCGCTCCCTCTACCCGGCCGGA | |
| 91 | CCCGGCTCTGCCCCCGCGCCCAAGCCCCACCAAGCCCCCCGCCCT | |
| 136 | CCCGCCGCGGTCCCAGCCCAGGGCGCGGCCGCAACCAGCACCATG | |
| | Met | |
| 181 | CGCCCGGTAGCCCTGCTGCTCCTGCCCTCGCTGCTGGCGCTCCTG | |
| | ArgProValAlaLeuLeuLeuLeuProSerLeuLeuAlaLeuLeu | |
| 226 | GCTCACGGACTCTCTTTAGAGGCCCCAACCGTGGGGAAAGGACAA | |
| | AlaHisGlyLeuSerLeuGluAlaProThrValGlyLysGlyGln | |
| 271 | GCCCCAGGCATCGAGGAGACAGATGGCGAGCTGACAGCAGCCCCC | |
| | AlaProGlyIleGluGluThrAspGlyGluLeuThrAlaAlaPro | |
| 316 | ACACCTGAGCAGCCAGAACGAGGCGTCCACTTTGTCACAACAGCC | |
| | ThrProGluGlnProGluArgGlyValHisPheValThrThrAla | |
| 361 | CCCACCTTGAAGCTGCTCAACCACCACCCGCTGCTTGAGGAATTC | |
| | ProThrLeuLysLeuLeuAsnHisHisProLeuLeuGluGluPhe | |
| 406 | CTACAAGAGGGGCTGGAAAAGGGAGATGAGGAGCTGAGGCCAGCA | |
| | LeuGlnGluGlyLeuGluLysGlyAspGluGluLeuArgProAla | |
| 451 | CTGCCCTTCCAGCCTGACCCACCTGCACCCTTCACCCCAAGTCCC | |
| | LeuProPheGlnProAspProProAlaProPheThrProSerPro | |
| 496 | CTTCCCCGCCTGGCCAACCAGGACAGCCGCCCTGTCTTTACCAGC | |
| | LeuProArgLeuAlaAsnGlnAspSerArgProValPheThrSer | |
| 541 | CCCACTCCAGCCATGGCTGCGGTACCCACTCAGCCCCAGTCCAAG | |
| | ProThrProAlaMetAlaAlaValProThrGlnProGlnSerLys | |
| 586 | GAGGGACCCTGGAGTCCGGAGTCAGAGTCCCCTATGCTTCGAATC | |
| | GluGlyProTrpSerProGluSerGluSerProMetLeuArgIle | |
| 631 | ACAGCTCCCCTACCTCCAGGGCCCAGCATGGCAGTGCCCACCCTA | |
| | ThrAlaProLeuProProGlyProSerMetAlaValProThrLeu | |
| 676 | GGCCCAGGGGAGATAGCCAGCACTACACCCCCCAGCAGAGCCTGG | |
| | GlyProGlyGluIleAlaSerThrThrProProSerArgAlaTrp | |
| 721 | ACACCAACCCAAGAGGGTCCTGGAGACATGGGAAGGCCGTGGGTT | |
| | ThrProThrGlnGluGlyProGlyAspMetGlyArgProTrpVal | |
| 766 | GCAGAGGTTGTGTCCCAGGGCGCAGGGATCGGGATCCAGGGGACC | |
| | AlaGluValValSerGlnGlyAlaGlyIleGlyIleGlnGlyThr | |
| 811 | ATCACCTCCTCCACAGCTTCAGGAGATGATGAGGAGACCACCACT | |
| | IleThrSerSerThrAlaSerGlyAspAspGluGluThrThrThr | |
| 856 | ACCACCACCATCATCACCACCACCATCACCACAGTCCAGACACCA | |
| | ThrThrThrIleIleThrThrThrIleThrThrValGlnThrPro | |
| 901 | GGTCAGCTACCTGCTGGCTTGCAGATGTGGAAATGGGGATGGGGG | |
| | GlyGlnLeuProAlaGlyLeuGlnMetTrpLysTrpGlyTrpGly | |
| 946 | AGGCTGCGGGGCCCCTAAAAGCCTGTCTCTGACACTGTGCCAGCC | |
| | ArgLeuArgGlyPro | (SEQ ID NO:26) |
| 991 | TGCCCTGCCCTTTGGCACCAAGGGCCAGCCTGCAGGAGGCATGTA | |
| 1036 | GATTGGACCCAGATAGACCTGAGCTCAAATCCTGATTCTTCAGCC | |
| 1081 | AAGTACAGTGGCTCATGCCTGTAATCCCAGCACTTTGGGAGGCAG | |
| 1126 | AGGCCAGTGGATCATCTGAGGTCAGGAGTTCAAGACCCTCCTGGC | |
| 1171 | CAACATGGCGAAACACCATCTCTACTAAAAATACAAAAATGAGCC | |
| 1216 | GGGCATGGTGGTGGGCACCTGTAATCCCAGCTACTCGGGAGGCTG | |
| 1261 | AGGCAGGAGAATCACTCAAACCTGGGAGGCAGAGGTTGCAGTGAG | |
| 1306 | CTGAGATTGCACCATTGCACTCCAGCCTGGGCAACAGAGCGAGAC | |
| 1351 | TCTGTCTCAAAAAAGAAAAAATCTTGATTCTTCCAACTATAACAT | |
| 1396 | GACCCTAGGAATTCTATTTAACATCTCATCTCTGAGCCTCATCTG | |
| 1441 | TAAAATGGCAATAAGAAAATAAACTTCTGGCTAGAAAAAAAA | (SEQ ID NO:27) |

Analysis of the protein sequence databases using the BLASTP and BLASTX computer programs revealed that the protein encoded by Clone 7520500.0.54_{—}4 (PROX 12) has 412 of 484 residues (85%) identical to, and 439 of 484 residues (90%) positive with, the 991 residue mouse seizure-related protein 6 precursor (type 2 ) (ACC:Q62269). The encoded protein also has 133 of 268 residues (49%) identical to, and 187 of 268 residues (69%) positive with, the 777 residue fragment of human KIAA0927 protein (ACC:BAA76771).

Analysis of the protein sequence databases using the BLASTP and BLASTX computer programs revealed that the protein encoded by Clone 7520500.0.21 (PROX 13) has 186 of 242 residues (76%) identical to, and 206 of 242 residues (85%) positive with, the 977 residue mouse seizure related protein 6 precursor (ACC:Q62269).

The proteins of the invention encoded by Clone 7520500.0.54_{—}1 (PROX 9); Clone 7520500.0.54_{—}2 (PROX 10); Clone 7520500.0.54_{—}3 (PROX 11); Clone 7520500.0.54_{—}4 (PROX 12); and Clone 7520500.0.21 (PROX 13), include the protein disclosed as being encoded by the ORFs described herein, as well as any mature protein arising therefrom as a result of post-translational modifications. Thus, the proteins of the invention encompass both a precursor and any active forms of the 7520500.0.54_{—}1, 7520500.0.54_{—}2, 7520500.0.54_{—}3, 7520500.0.54_{—}4 and 7520500.0.21 proteins.

Experimental results presented in Example 16 show that the various clones of the 7520500 family are prominently detected in two lung cancer cell lines, but not in normal lung cells. These results suggest that this clone may be used as a selective probe for detection or diagnosis of these cancers, and that the clones or their gene products may be useful targets in treatment of such cancers.

### PRO14 and PRO15 Nucleic Acids and Polypeptides

A PRO14 or PRO15 nucleic acid according to the invention includes the nucleic acid sequence represented in Clone 17941787.0.1 (PROX 14) and Clone 17941787.0.31 (PROX 15). These clones resemble each other in that the proteins they encode appear to be splice variants of one another. For example, there is a deletion of 19 amino acid residues in the protein encoded by Clone 17941787.0.1 (PROX 14) beginning at residue 26, as compared to Clone 17941787.0.31 (PROX 15). In addition, Clone 17941787.0.31 (PROX 15) is extended to a much further degree at the carboxyl-terminus, than is Clone 17941787.0.1 (PROX 14).

The nucleic acid representative of Clone 17941787.0.1 (PROX 14) was found in mammary gland, as well as in fetal kidney and pituitary gland. A representation of the nucleotide sequence of Clone 17941787.0.1 is presented in Table 15 and includes a nucleotide sequence (SEQ ID NO:27) of 3336 bp. This nucleotide sequence has an open reading frame (ORF) encoding a polypeptide of 840 amino acid residues (SEQ ID NO:28) with a predicted molecular weight of 93122 Daltons. The start codon is located at nucleotides 120-122; and the stop codon is located at nucleotides 2640-2642. The protein (SEQ ID NO:28) encoded by Clone 17941787.0.1 was predicted by the PSORT computer program to be localized in the plasma membrane. The PSORT and SignalP computer programs also predicted that there is a cleavable signal peptide, with the most likely cleavage site located between residues 27 and 28, at the VYA-CG. The nucleic acid (SEQ ID NO:27) and amino acid (SEQ ID NO:28) sequences of Clone 17941787.0.1 (PROX 14) are shown below in Table 15.

TABLE 15
Clone 17941787.0.1
Translated Protein - Frame: 3 - Nucleotide 120 to 2639

| | | |
|---|---|---|
| 1 | CGCCGGTGGCTCGGCGGCGGCGGCGGCGGCGGCGGCGGCGGCGGC | |
| 46 | GGCGGCGTCGTCTACCTCCAGCTCCTCCTCCCTCCTCCTCCGTCT | |
| 91 | CCTCCTCTCTCTCTCCATCTGCTGTGGTTATGGCCTGTCGCTGGA | |
| | MetAlaCysArgTrpS | |
| 136 | GCACAAAAGAGTCTCCGCGGTGGAGGTCTGCGTTGCTCTTGCTTT | |
| | erThrLysGluSerProArgTrpArgSerAlaLeuLeuLeuLeuP | |
| 181 | TCCTCGCTGGGGTGTACGCTTGTGGAGAGACTCCAGAGCAAATAC | |
| | heLeuAlaGlyValTyrAlaCysGlyGluThrProGluGlnIleA | |
| 226 | GAGCACCAAGTGGCATAATCACAAGCCCAGGCTGGCCTTCTGAAT | |
| | rgAlaProSerGlyIleIleThrSerProGlyTrpProSerGluT | |
| 271 | ATCCTGCAAAAATCAACTGTAGCTGGTTCATAAGGGCAAACCCAG | |
| | yrProAlaLysIleAsnCysSerTrpPheIleArgAlaAsnProG | |
| 316 | GCGAAATCATTACTATAAGTTTTCAGGATTTTGATATTCAAGGAT | |
| | lyGluIleIleThrIleSerPheGlnAspPheAspIleGlnGlyS | |
| 361 | CCAGAAGGTGCAATTTGGACTGGTTGACAATAGAAACATACAAGA | |
| | erArgArgCysAsnLeuAspTrpLeuThrIleGluThrTyrLysA | |
| 406 | ATATTGAAAGTTACAGAGCTTGTGGTTCCACAATTCCACCTCCGT | |
| | snIleGluSerTyrArgAlaCysGlySerThrIleProProProT | |
| 451 | ATATCTCTTCACAAGACCACATCTGGATTAGGTTTCATTCGGATG | |
| | yrIleSerSerGlnAspHisIleTrpIleArgPheHisSerAspA | |
| 496 | ACAACATCTCTAGAAAGGGTTTCAGACTGGCATATTTTTCAGGGA | |
| | spAsnIleSerArgLysGlyPheArgLeuAlaTyrPheSerGlyL | |
| 541 | AATCTGAGGAACCAAATTGTGCTTGTGATCAGTTTCGTTGTGGTA | |
| | ysSerGluGluProAsnCysAlaCysAspGlnPheArgCysGlyA | |
| 586 | ATGGAAAGTGTATACCAGAAGCCTGGAAATGCAATAACATGGATG | |
| | snGlyLysCysIleProGluAlaTrpLysCysAsnAsnMetAspG | |
| 631 | AATGTGGAGATAGTTCCGATGAAGAGATCTGTGCCAAAGAAGCAA | |
| | luCysGlyAspSerSerAspGluGluIleCysAlaLysGluAlaA | |
| 676 | ATCCTCCAACTGCTGCTGCTTTTCAACCCTGTGCTTACAACCAGT | |
| | snProProThrAlaAlaAlaPheGlnProCysAlaTyrAsnGlnP | |
| 721 | TCCAGTGTTTATCCCGTTTTACCAAAGTTTACACTTGCCTCCCCG | |
| | heGlnCysLeuSerArgPheThrLysValTyrThrCysLeuProG | |
| 766 | AATCTTTAAAATGTGATGGGAACATTGACTGCCTTGACCTAGGAG | |
| | luSerLeuLysCysAspGlyAsnIleAspCysLeuAspLeuGlyA | |
| 811 | ATGAGATAGACTGTGATGTGCCAACATGTGGGCAATGGCTAAAAT | |
| | spGluIleAspCysAspValProThrCysGlyGlnTrpLeuLysT | |
| 856 | ATTTTTATGGTACTTTTAATTCTCCCAATTATCCAGACTTTTATC | |
| | yrPheTyrGlyThrPheAsnSerProAsnTyrProAspPheTyrP | |
| 901 | CTCCTGGAAGCAATTGCACCTGGTTAATAGACACTGGTGATCACC | |
| | roProGlySerAsnCysThrTrpLeuIleAspThrGlyAspHisA | |
| 946 | GTAAAGTCATTTTACGCTTCACTGACTTTAAACTTGATGGTACTG | |
| | rgLysValIleLeuArgPheThrAspPheLysLeuAspGlyThrG | |
| 991 | GTTATGGTGATTATGTCAAAATATATGATGGATTAGAGGAGAATC | |
| | lyTyrGlyAspTyrValLysIleTyrAspGlyLeuGluGluAsnP | |
| 1036 | CACACAAGCTTTTGCGTGTGTTGACAGCTTTTGATTCTCATGCAC | |
| | roHisLysLeuLeuArgValLeuThrAlaPheAspSerHisAlaP | |
| 1081 | CTCTTACAGTTGTTTCTTCTTCTGGACAGATAAGGGTACATTTTT | |
| | roLeuThrValValSerSerSerGlyGlnIleArgValHisPheC | |
| 1126 | GTGCTGATAAAGTGAATGCTGCAAGGGGATTTAATGCTACTTACC | |
| | ysAlaAspLysValAsnAlaAlaArgGlyPheAsnAlaThrTyrG | |
| 1171 | AAGTAGATGGGTTCTGTTTGCCATGGGAAATACCCTGTGGAGGTA | |
| | lnValAspGlyPheCysLeuProTrpGluIleProCysGlyGlyA | |
| 1216 | ACTGGGGGTGTTATACTGAGCAGCAGCGTTGTGATGGGTATTGGC | |
| | snTrpGlyCysTyrThrGluGlnGlnArgCysAspGlyTyrTrpH | |
| 1261 | ATTGCCCAAATGGAAGGGATGAAACCAATTGTACCATGTGCCAGA | |
| | isCysProAsnGlyArgAspGluThrAsnCysThrMetCysGlnL | |
| 1306 | AGGAAGAATTTCCATGTTCCCGAAATGGTGTCTGTTATCCTCGTT | |
| | ysGluGluPheProCysSerArgAsnGlyValCysTyrProArgS | |
| 1351 | CTGATCGCTGCAACTACCAGAATCATTGCCCAAATGGCTCAGATG | |
| | erAspArgCysAsnTyrGlnAsnHisCysProAsnGlySerAspG | |
| 1396 | AAAAAAACTGCTTTTTTTGCCAACCAGGAAATTTCCATTGTAAAA | |
| | luLysAsnCysPhePheCysGlnProGlyAsnPheHisCysLysA | |
| 1441 | ACAATCGTTGTGTGTTTGAAAGTTGGGTGTGTGATTCTCAAGATG | |
| | snAsnArgCysValPheGluSerTrpValCysAspSerGlnAspA | |
| 1486 | ACTGTGGTGATGGCAGCGATGAAGAAAATTGCCCAGTAATCGTGC | |
| | spCysGlyAspGlySerAspGluGluAsnCysProValIleValP | |
| 1531 | CTACAAGAGTCATCACTGCTGCCGTCATAGGGAGCCTCATCTGTG | |
| | roThrArgValIleThrAlaAlaValIleGlySerLeuIleCysG | |
| 1576 | GCCTGTTACTCGTCATAGCATTGGGATGTACTTGTAAGCTTTATT | |
| | lyLeuLeuLeuValIleAlaLeuGlyCysThrCysLysLeuTyrS | |
| 1621 | CTCTGAGAATGTTTGAAAGAAGATCATTTGAAACACAGTTGTCAA | |
| | erLeuArgMetPheGluArgArgSerPheGluThrGlnLeuSerA | |
| 1666 | GAGTGGAAGCAGAATTGTTAAGAAGAGAAGCTCCTCCCTCGTATG | |
| | rgValGluAlaGluLeuLeuArgArgGluAlaProProSerTyrG | |
| 1711 | GACAATTGATTGCTCAGGGTTTAATTCCACCAGTTGAAGATTTTC | |
| | lyGlnLeuIleAlaGlnGlyLeuIleProProValGluAspPheP | |
| 1756 | CTGTTTGTTCACCTAATCAGGCTTCTGTTTTGGAAAATCTGAGGC | |
| | roValCysSerProAsnGlnAlaSerValLeuGluAsnLeuArgL | |
| 1801 | TAGCGGTACGATCTCAGCTTGGATTTACTTCAGTCAGGCTTCCTA | |
| | euAlaValArgSerGlnLeuGlyPheThrSerValArgLeuProM | |
| 1846 | TGGCAGGCAGATCAAGCAACATTTGGAACCGTATTTTTAATTTTG | |
| | etAlaGlyArgSerSerAsnIleTrpAsnArgIlePheAsnPheA | |
| 1891 | CAAGATCACGTCATTCTGGGTCATTGGCTTTGGTCTCAGCAGATG | |
| | laArgSerArgHisSerGlySerLeuAlaLeuValSerAlaAspG | |
| 1936 | GAGATGAGGTTGTCCCTAGTCAGAGTACCAGTAGAGAACCTGAGA | |
| | lyAspGluValValProSerGlnSerThrSerArgGluProGluA | |
| 1981 | GAAATCATACTCACAGAAGTTTGTTTTCCGTGGAGTCTGATGATA | |
| | rgAsnHisThrHisArgSerLeuPheSerValGluSerAspAspT | |
| 2026 | CAGACACAGAAAATGAGAGAAGAGATATGGCAGGAGCATCTGGTG | |
| | hrAspThrGluAsnGluArgArgAspMetAlaGlyAlaSerGlyG | |
| 2071 | GGGTTGCAGCTCCTTTGCCTCAAAAAGTCCCTCCCACAACGGCAG | |
| | lyValAlaAlaProLeuProGlnLysValProProThrThrAlaV | |
| 2116 | TAGAAGCGACAGTAGGAGCATGTGCAAGTTCCTCAACTCAGAGTA | |
| | alGluAlaThrValGlyAlaCysAlaSerSerSerThrGlnSerT | |
| 2161 | CCCGAGGTGGTCATGCAGATAATGGAAGGGATGTGACAAGTGTGG | |
| | hrArgGlyGlyHisAlaAspAsnGlyArgAspValThrSerValG | |
| 2206 | AACCCCCAAGTGTGAGTCCAGCACGTCACCAGCTTACAAGTGCAC | |
| | luProProSerValSerProAlaArgHisGlnLeuThrSerAlaL | |
| 2251 | TCAGTCGTATGACTCAGGGGCTACGCTGGGTACGTTTTACATTAG | |
| | euSerArgMetThrGlnGlyLeuArgTrpValArgPheThrLeuG | |
| 2296 | GACGATCAAGTTCCCTAAGTCAGAACCAGAGTCCTTTGAGACAAC | |
| | lyArgSerSerSerLeuSerGlnAsnGlnSerProLeuArgGlnL | |
| 2341 | TTGATAATGGGGTAAGTGGAAGAGAAGATGATGATGATGTTGAAA | |
| | euAspAsnGlyValSerGlyArgGluAspAspAspAspValGluM | |
| 2386 | TGCTAATTCCAATTTCTGATGGATCTTCAGACTTTGATGTGAATG | |
| | etLeuIleProIleSerAspGlySerSerAspPheAspValAsnA | |
| 2431 | ACTGCTCCAGACCTCTTCTTGATCTTGCCTCAGATCAAGGACAAG | |
| | spCysSerArgProLeuLeuAspLeuAlaSerAspGlnGlyGlnG | |
| 2476 | GGCTTAGACAACCATATAATGCAACAAATCCTGGAGTAAGGCCAA | |
| | lyLeuArgGlnProTyrAsnAlaThrAsnProGlyValArgProS | |
| 2521 | GTAATCGAGATGGCCCCTGTGAGCGCTGTGGTATTGTCCACACTG | |
| | erAsnArgAspGlyProCysGluArgCysGlyIleValHisThrA | |
| 2566 | CCCAGATACCAGACACTTGCTTAGAAGTAACACTGAAAAACGAAA | |
| | laGlnIleProAspThrCysLeuGluValThrLeuLysAsnGluT | |
| 2611 | CGAGTGATGATGAGGCTTTGTTACTTTGTTAGGTACGAATCACAT | |
| | hrSerAspAspGluAlaLeuLeuLeuCys | (SEQ ID NO:28) |
| 2656 | AAGGGAGATTGTATACAAGTTGGAGCAATATCCATTTATTATTTT | |
| 2701 | GTAACTTTACAGTTAAACTAGTTTTAGTTTAAAAAGAAAAAATGC | |
| 2746 | AGGGTGATTTCTTATTATTATATGTTAGCCTGCATGGTTAAATTC | |
| 2791 | GACAACTTGTAACTCTATGAACTTAGAGTTTACTATTTTAGCAGC | |
| 2836 | TAAAAATGCATCACATATTGCATATTGTTCAATAATGGTCCTTTC | |
| 2881 | ATTTGTTTCTGATTGTTTTCATCCTGATACTGTAGTTCACTGTAG | |
| 2926 | AAATGTGGCTGCTGAAACTCATTTGATTGTCATTTTTATCTATCC | |
| 2971 | TATGTTAAATGGTTTGTTTTTACAAAATAATACCTTATTTTAATT | |
| 3016 | GAAACGTTTATGCTTTTGCCAAGCACATCTTGTAACTTAATATAG | |
| 3061 | CTAGATGTTAAGGTTGTTAATGTACCAAAAAAAAAAAACCTTATA | |
| 3106 | CTCACCTGCGTTTTCATTTGTTTGACATTTGTCTATTATTGGATA | |
| 3151 | TCATTATCATATGAACTTGTCAGTGGGAAACAAACTGTCTAAAAA | |
| 3196 | TTTATCTCTTACGTTTAACATACAATCATGTGAGATTTAGGCAGA | |
| 3241 | GTTCGATAAATTACTGGCAAAAACAAAACTCATTTATAAAGATTT | |
| 3286 | TCTAATGTTGACTTTAATACTCTAACATGGTACAAACCANATGGT | |
| 3331 | AAAATC | (SEQ ID NO:27) |

BLASTP and BLASTX computer programs reveal that the protein encoded by Clone 17941787.0.1 (PROX14) has 816 of 820 residues (99%) identical to, and 818 of 820 residues (99%) positive with the 859 residue human ST7 protein (SPTREMBL-ACC:Q9Y561; deposited after the filing date of the present application), a putative transmembrane protein with altered expression in some human transformed and tumor-derived cell lines. In addition, the encoded protein was also found to have 301 of 586 residues (51%) identical to, and 397 of 586 residues (67%) positive with, the 770 residue human LDL receptor-related protein 105 (ACC:O75074). Furthermore, the encoded protein was found to have 816 of 820 residues 99%) identical to, and 818 of 820 residues (99%) positive with, a human 859 residue polypeptide identified by the signal sequence trap method (PCT Publication WO 9918126-A1, published Apr. 15, 1999).

RNA homologous to Clone 17941787.0.31 is found in mammary gland, as well as in fetal kidney and pituitary gland. A representation of the nucleotide sequence of clone 17941787.0.31 (PROX 15) is provided in Table 16 and includes a nucleotide sequence (SEQ ID NO:29) of 1498 bp. This nucleotide sequence has an open reading frame (ORF) encoding of polypeptide of 449 amino acid residues (SEQ ID NO:30) with a predicted molecular weight of 50654 Daltons. The start codon is located at nucleotides 120-122; and the stop codon is located at nucleotides 1467-1469. The protein (SEQ ID NO:30) encoded by Clone 17941787.0.31 was predicted by the PSORT computer program to be localized extracellularly with a certainty of 0.5660. The PSORT and SignalP computer programs predicted that there is a cleavable signal peptide, with the most likely cleavage site located between residues 27 and 28, at sequence VYG-NG. The nucleic acid (SEQ ID NO:29) and amino acid (SEQ ID NO:30) sequences of Clone 17941787.0.31 (PROX 15) are shown below in Table 16.

TABLE 16
Clone 17941787-0-31
Translated Protein - Frame: 3 - Wucleotide 120 to 1466

| | | |
|---|---|---|
| 1 | CGCCGGTGGCTCGGCGGCGGCGGCGGCGGCGGCGGCGGCGGCGGC | |
| 46 | GGCGGCGTCGTCTACCTCCAGCTCCTCCTCCCTCCTCCTCCGTCT | |
| 91 | CCTCCTCTCTCTCTCCATCTGCTGTGGTTATGGCCTGTCGCTGGA | |
| | MetAlaCysArgTrpS | |
| 136 | GCACAAAAGAGTCTCCGCGGTGGAGGTCTGCGTTGCTCTTGCTTT | |
| | erThrLysGluSerProArgTrpArgSerAlaLeuLeuLeuLeuP | |
| 181 | TCCTCGCTGGGGTGTACGGAAATGGTGCTCTTGCAGAACATTCTG | |
| | heLeuAlaGlyValTyrGlyAsnGlyAlaLeuAlaGluHisSerG | |
| 226 | AAAATGTGCATATTTCAGGAGTGTCAACTGCTTGTGGAGAGACTC | |
| | luAsnValHisIleSerGlyValSerThrAlaCysGlyGluThrP | |
| 271 | CAGAGCAAATACGAGCACCAAGTGGCATAATCACAAGCCCAGGCT | |
| | roGluGlnIleArgAlaProSerGlyIleIleThrSerProGlyT | |
| 316 | GGCCTTCTGAATATCCTGCAAAAATCAACTGTAGCTGGTTCATAA | |
| | rpProSerGluTyrProAlaLysIleAsnCysSerTrpPheIleA | |
| 361 | GGGCAAACCCAGGCGAAATCATTACTATAAGTTTTCAGGATTTTG | |
| | rgAlaAsnProGlyGluIleIleThrIleSerPheGlnAspPheA | |
| 406 | ATATTCAAGGATCCAGAAGGTGCAATTTGGACTGGTTGACAATAG | |
| | spIleGlnGlySerArgArgCysAsnLeuAspTrpLeuThrIleG | |
| 451 | AAACATACAAGAATATTGAAAGTTACAGAGCTTGTGGTTCCACAA | |
| | luThrTyrLysAsnIleGluSerTyrArgAlaCysGlySerThrI | |
| 496 | TTCCACCTCCGTATATCTCTTCACAAGACCACATCTGGATTAGGT | |
| | leProProProTyrIleSerSerGlnAspHisIleTrpIleArgP | |
| 541 | TTCATTCGGATGACAACATCTCTAGAAAGGGTTTCAGACTGGCAT | |
| | heHisSerAspAspAsnIleSerArgLysGlyPheArgLeuAlaT | |
| 586 | ATTTTTCAGGGAAATCTGAGGAACCAAATTGTGCTTGTGATCAGT | |
| | yrPheSerGlyLysSerGluGluProAsnCysAlaCysAspGlnP | |
| 631 | TTCGTTGTGGTAATGGAAAGTGTATACCAGAAGCCTGGAAATGTA | |
| | heArgCysGlyAsnGlyLysCysIleProGluAlaTrpLysCysA | |
| 676 | ATAACATGGATGAATGTGGAGATAGTTCCGATGAAGAGATCTGTG | |
| | snAsnMetAspGluCysGlyAspSerSerAspGluGluIleCysA | |
| 721 | CCAAAGAAGCAAATCCTCCAACTGCTGCTGCTTTTCAACCCTGTG | |
| | laLysGluAlaAsnProProThrAlaAlaAlaPheGlnProCysA | |
| 766 | CTTACAACCAGTTCCAGTGTTTATCCCGTTTTACCAAAGTTTACA | |
| | laTyrAsnGlnPheGlnCysLeuSerArgPheThrLysValTyrT | |
| 811 | CTTGCCTCCCCGAATCTTTAAAATGTGATGGGAACATTGACTGCC | |
| | hrCysLeuProGluSerLeuLysCysAspGlyAsnIleAspCysL | |
| 856 | TTGACCTAGGAGATGAGATAGACTGTGATGTGCCAACATGTGGGC | |
| | euAspLeuGlyAspGluIleAspCysAspValProThrCysGlyG | |
| 901 | AATGGCTAAAATATTTTTATGGTACTTTTAATTCTCCCAATTATC | |
| | lnTrpLeuLysTyrPheTyrGlyThrPheAsnSerProAsnTyrp | |
| 946 | CAGACTTTTATCCTCCTGGAAGCAATTGCACCTGGTTAATAGACA | |
| | roAspPheTyrProProGlySerAsnCysThrTrpLeuIleAspT | |
| 991 | CTGGTGATCACCGTAAAGTCATTTTACGCTTCACTGACTTTAAAC | |
| | hrGlyAspHisArgLysValIleLeuArgPheThrAspPheLysL | |
| 1036 | TTGATGGTACTGGTTATGGTGATTATGTCAAAATATATGATGGAT | |
| | euAspGlyThrGlyTyrGlyAspTyrValLysIleTyrAspGlyL | |
| 1081 | TAGAGGAGAATCCACACAAGCTTTTGCGTGTGTTGACAGCTTTTG | |
| | euGluGluAsnProHisLysLeuLeuArgValLeuThrAlaPheA | |
| 1126 | ATTCTCATGCACCTCTTACAGTTGTTTCTTCTTCTGGACAGATAA | |
| | spSerHisAlaProLeuThrValValSerSerSerGlyGlnIleA | |
| 1171 | GGGTACATTTTTGTGCTGATAAAGTGAATGCTGCAAGGGGATTTA | |
| | rgValHisPheCysAlaAspLysValAsnAlaAlaArgGlyPheA | |
| 1216 | ATGCTACTTACCAAGTAGATGGGTTCTGTTTGCCATGGGAAATAC | |
| | snAlaThrTyrGlnValAspGlyPheCysLeuProTrpGluIleP | |
| 1261 | CCTGTGGAGGTAACTGGGGGTGTTATACTGAGCAGCAGCGTCGTG | |
| | roCysGlyGlyAsnTrpGlyCysTyrThrGluGlnGlnArgArgA | |
| 1306 | ATGGGTATTGGCATTGCCCAAATGGAAGGGATGAAACCAATTGTA | |
| | spGlyTyrTrpHisCysProAsnGlyArgAspGluThrAsnCysT | |
| 1351 | CCATGTGCCAGAAGGAAGAATTTCCATGTTCCCGAAATGGTGTCT | |
| | hrMetCysGlnLysGluGluPheProCysSerArgAsnGlyValC | |
| 1396 | GTTATCCTCGTTCTGATCGCTGCAACTACCAGAATCATTGCCCAA | |
| | ysTyrProArgSerAspArgCysAsnTyrGlnAsnHisCysProA | |
| 1441 | ATGGCAAACAGAACCCATCTACTTGGTAAGTAGCATTAAATCCCC | |
| | snGlyLysGlnAsnProSerThrTrp | (SEQ ID NO:30) |
| 1486 | TTGCAGCATTCAC | (SEQ ID NO:29) |

BLASTP and BLASTX analyses reveal that the protein encoded by Clone 17941787.0.31 (PROX 15) has 441 of 442 residues (99%) identical to, and 441 of 442 residues (99%) positive with, the 859 residue human ST7 protein (ACC:AAD44360), a putative transmembrane protein with altered expression in some human transformed and tumor-derived cell lines. In addition, the protein encoded by Clone 1791787.0.31 was also found to have 301 of 586 residues (51%) identical to, and 397 of 586 residues (67%) positive with, the 770 residue human LDL receptor-related protein 105 (ACC:O75074). Furthermore, the encoded protein has 441 of 442 residues (99%) identical to and positive with, a human 859 residue polypeptide identified by the signal sequence trap method (PCT Publication WO 9918126A1, filed Apr. 15, 1999).

The proteins of the invention encoded by Clone 17941787.0. 1 (PROX 14) and Clone 17941787.0.31 (PROX 15) include the protein disclosed as being encoded by the ORFs described herein, as well as any mature protein arising therefrom as a result of post-translational modifications. Thus, the proteins of the invention encompass both a precursor and any active forms of the 17941787.0.1 and 17941787.0.31 proteins.

Experimental results presented in Example 16 show that, relative to cells from normal tissues, Clone 17941787 is strongly over-expressed in prostate cancer, ovarian cancer, breast cancer, lung cancer, renal cancer, CNS cancer, and pancreatic cancer cell lines. These results suggest that this clone may be used as a selective probe for detection or diagnosis of these cancers, and that the clones or their gene products may be useful targets in treatment of such cancers.

### PRO16 and PRO17 Nucleic Acids and Polypeptides

A PRO16 or PRO17 nucleic acid according to the invention includes the nucleic acid sequence represented in Clone 16467945.0.85 (PROX 16) and Clone 16467945.0.88 (PROX 17). These clones resemble each other in that the proteins they encode appear to be splice variants of one another. They are essentially identical at the amino-terminal portion, become dissimilar at the carboxyl-terminal portion of the shorter protein (i e., the protein encoded by Clone 16467945.0.85), and then only Clone 16467945.0.88 continues with an extended carboxyl-terminal sequence.

RNA homologous to Clone 16467945.0.85 (PROX 16) and Clone 16467945.0.88 (PROX 17) found in fetal lung, testis, and fetal kidney.

A representation of the nucleotide sequence of Clone16467945.0.85 (PROX 16) is presented in Table 17 and includes a nucleotide sequence (SEQ ID NO:3 1) of 691 bp. This nucleotide sequence has an open reading frame (ORF) encoding a polypeptide of 123 amino acid residues (SEQ ID NO:32) with a predicted molecular weight of 13844 Daltons. The start codon is located at nucleotides 203-205; and the stop codon is located at nucleotides 572-574. The protein (SEQ ID NO:32) encoded by Clone 16467945.0.85 (PROX 16) was predicted by the PSORT computer program to be localized extracellularly with a certainty of 0.7475. The PSORT and SignalP computer programs also predicted that there is a cleavable signal peptide, with the most likely cleavage site located between residues 19 and 20, at the sequence AAA-EY. The nucleic acid (SEQ ID NO:31) and amino acid (SEQ ID NO:32) sequences of Clone 16467945.0.85 (PROX 16) are shown below in Table 17.

TABLE 17
Clone 16467945.0.85
Translated Protein - Frame: 2 - Nucleotide 203 to 571

| | | |
|---|---|---|
| 1 | GGGAGGGGGCTCCGGGCGCCGCGCAGCAGACCTGCTCCGGCCGCG | |
| 46 | CGCCTCGCCGCTGTCCTCCGGGAGCGGCAGCAGTAGCCCGGGCGG | |
| 91 | CGAGGGCTGGGGGTTCCTCGAGACTCTCAGAGGGGCGCCTCCCAT | |
| 136 | CGGCGCCCACCACCCCAACCTGTTCCTCGCGCGCCACTGCGCTGC | |
| 181 | GCCCCAGGACCCGCTGCCCAACATGGATTTTCTCCTGGCGCTGGT | |
| | MetAspPheLeuLeuAlaLeuVa | |
| 226 | GCTGGTATCCTCGCTCTACCTGCAGGCGGCCGCCGAGTACGACGG | |
| | lLeuValSerSerLeuTyrLeuGlnAlaAlaAlaGluTyrAspGl | |
| 271 | GAGGTGGCCCAGGCAAATAGTGTCATCGATTGGCCTATGTCGTTA | |
| | yArgTrpProArgGlnIleValSerSerIleGlyLeuCysArgTy | |
| 316 | TGGTGGGAGGATTGACTGCTGCTGGGGCTGGGCTCGCCAGTCTTG | |
| | rGlyGlyArgIleAspCysCysTrpGlyTrpAlaArgGlnSerTr | |
| 361 | GGGACAGTGTCAGCCTTTCTACGTCTTAAGGCAGAGAATAGCCAG | |
| | pGlyGlnCysGlnProPheTyrValLeuArgGlnArgIleAlaAr | |
| 406 | GATAAGGTGCCAGCTCAAAGCTGTGTGCCAACCACGATGCAAACA | |
| | gIleArgCysGlnLeuLysAlaValCysGlnProArgCysLysHi | |
| 451 | TGGTGAATGTATCGGGCCAAACAAGTGCAAGTGTCATCCTGGTTA | |
| | sGlyGluCysIleGlyProAsnLysCysLysCysHisProGlyTy | |
| 496 | TGCTGGAAAAACCTGTAATCAAGCCGTAGGTTTTGAAAGATGTAT | |
| | rAlaGlyLysThrCysAsnGlnAlaValGlyPheGluArgCysMe | |
| 541 | GGTTCCAGCCGGGCGCCGTGGCTCTACCCTGTAATCCCAGCACTT | |
| | tValProAlaGlyArgArgGlySerThrLeu | (SEQ ID NO:32) |
| 586 | TGGAAGGCCGAGGCGGGCGGATCACGAGGTCAGGATATCGAGACC | |
| 631 | ATCCTGGCTAACACGGTGAAACCCCATCTCTACTAAAAATACAAA | |
| 676 | AAAAAAAAAAAAAAAA | (SEQ ID NO:31) |

Analysis of the sequence databases using the BLASTP and BLASTX computer programs revealed that the protein encoded by Clone 16467945.0.85 (PROX 16) has 77 of 131 residues (58%) identical to, and 83 of 131 residues (63%) positive with, the 509 residue human PRO334 protein. In addition, the encoded protein was also found to have 21 of 47 residues (44%) identical to, and 27 of 47 residues (57%) positive with, the 700 residue mouse hedgehog-interacting protein (ACC: AAD31172).

A representation of the nucleotide sequence of Clone16467945.0.88 (PROX 17) is given in Table 18 and includes a nucleotide sequence (SEQ ID NO:33) of 2112 bp. This nucleotide sequence has an open reading frame (ORF) encoding a polypeptide of 582 amino acid residues (SEQ ID NO:34) with a predicted molecular weight of 63992 Daltons. The start codon is located at nucleotides 203-205; and the stop codon is located at nucleotides 1949-1951. The protein (SEQ ID NO:34) encoded by Clone 16467945.0.88 (PROX 17) was predicted by the PSORT computer program to be localized extracellularly with a certainty of 0.7475. The PSORT and SignalP computer programs also predicted that there is a cleavable signal peptide, with the most likely cleavage site located between residues 19 and 20, at the sequence AAA-EF. The nucleic acid (SEQ ID NO:33) and amino acid (SEQ ID NO:34) sequences of Clone 16467945.0.88 (PROX 17) are shown below in Table 18.

TABLE 18
Clone 16467945.0.88
Translated Protein - Frame: 2 - Nucleotide 203 to 1948

| | | |
|---|---|---|
| 1 | GGGAGGGGGCTCCGGGCGCCGCGCAGCAGACCTGCTCCGGCCGCG | |
| 46 | CGCCTCGCCGCTGTCCTCCGGGAGCGGCAGCAGTAGCCCGGGCGG | |
| 91 | CGAGGGCTGGGGGTTCCTCGAGACTCTCAGAGGGGCGCCTCCCAT | |
| 136 | CGGCGCCCACCACCCCAACCTGTTCCTCGCGCGCCACTGCGCTGC | |
| 181 | GCCCCAGGACCCGCTGCCCAACATGGATTTTCTCCTGGCGCTGGT | |
| | MetAspPheLeuLeuAlaLeuVa | |
| 226 | GCTGGTATCCTCGCTCTACCTGCAGGCGGCCGCCGAGTTCGACGG | |
| | lLeuValSerSerLeuTyrLeuGlnAlaAlaAlaGluPheAspGl | |
| 271 | GAGGTGGCCCAGGCAAATAGTGTCATCGATTGGCCTATGTCGTTA | |
| | yArgTrpProArgGlnIleValSerSerIleGlyLeuCysArgTy | |
| 316 | TGGTGGGAGGATTGACTGCTGCTGGGGCTGGGCTCGCCAGTCTTG | |
| | rGlyGlyArgIleAspCysCysTrpGlyTrpAlaArgGlnSerTr | |
| 361 | GGGACAGTGTCAGCCTTTCTACGTCTTAAGGCAGAGAATAGCCAG | |
| | pGlyGlnCysGlnProPheTyrValLeuArgGlnArgIleAlaAr | |
| 406 | GATAAGGTGCCAGCTCAAAGCTGTGTGCCAACCACGATGCAAACA | |
| | gIleArgCysGlnLeuLysAlaValCysGlnProArgCysLysHi | |
| 451 | TGGTGAATGTATCGGGCCAAACAAGTGCAAGTGTCATCCTGGTTA | |
| | sGlyGluCysIleGlyProAsnLysCysLysCysHisProGlyTy | |
| 496 | TGCTGGAAAAACCTGTATTCAAGTTTTAAATGAGTGTGGCCTGAA | |
| | rAlaGlyLysThrCysIleGlnValLeuAsnGluCysGlyLeuLy | |
| 541 | GCCCCGGCCCTGTAAGCACAGGTGCATGAACACTTACGGCAGCTA | |
| | sProArgProCysLysHisArgCysMetAsnThrTyrGlySerTy | |
| 586 | CAAGTGCTACTGTCTCAACGGATATATGCTCATGCCGGATGGTTC | |
| | rLysCysTyrCysLeuAsnGlyTyrMetLeuNetProAspGlySe | |
| 631 | CTGCTCAAGTGCCCTGACCTGCTCCATGGCAAACTGTCAGTATGG | |
| | rCysSerSerAlaLeuThrCysSerMetAlaAsnCysGlnTyrGl | |
| 676 | CTGTGATGTTGTTAAAGGACAAATACGGTGCCAGTGCCCATCCCC | |
| | yCysAspValValLysGlyGlnIleArgCysGlnCysProSerPr | |
| 721 | TGGCCTGCAGCTGGCTCCTGATGGGAGGACCTGTGTAGATGTTGA | |
| | oGlyLeuGlnLeuAlaProAspGlyArgThrCysValAspValAs | |
| 766 | TGAATGTGCTACAGGAAGAGCCTCCTGCCCTAGATTTAGGCAATG | |
| | pGluCysAlaThrGlyArgAlaSerCysProArgPheArgGlnCy | |
| 811 | TGTCAACACTTTTGGGAGCTACATCTGCAAGTGTCATAAAGGCTT | |
| | sValAsnThrPheGlySerTyrIleCysLysCysHisLysGlyPh | |
| 856 | CGATCTCATGTATATTGGAGGCAAATATCAATGTCATGACATAGA | |
| | eAspLeuMetTyrIleGlyGlyLysTyrGlnCysHisAspIleAs | |
| 901 | CGAATGCTCACTTGGTCAGTATCAGTGCAGCAGCTTTGCTCGATG | |
| | pGluCysSerLeuGlyGlnTyrGlnCysSerSerPheAlaArgCy | |
| 946 | TTATAACGTACGTGGGTCCTACAAGTGCAAATGTAAAGAAGGATA | |
| | sTyrAsnValArgGlySerTyrLysCysLysCysLysGluGlyTy | |
| 991 | CCAGGGTGATGGACTGACTTGTGTGTATATCCCAAAAGTTATGAT | |
| | rGlnGlyAspGlyLeuThrCysValTyrIleProLysValMetIl | |
| 1036 | TGAACCTTCAGGTCCAATTCATGTACCAAAGGGAAATGGTACCAT | |
| | eGluProSerGlyProIleHisValProLysGlyAsnGlyThrIl | |
| 1081 | TTTAAAGGGTGACACAGGAAATAATAATTGGATTCCTGATGTTGG | |
| | eLeuLysGlyAspThrGlyAsnAsnAsnTrpIleProAspValGl | |
| 1126 | AAGTACTTGGTGGCCTCCGAAGACACCATATATTCCTCCTATCAT | |
| | ySerThrTrpTrpProProLysThrProTyrIleProProIleIl | |
| 1171 | TACCAACAGGCCTACTTCTAAGCCAACAACAAGACCTACACCAAA | |
| | eThrAsnArgProThrSerLysProThrThrArgProThrProLy | |
| 1216 | GCCAACACCAATTCCTACTCCACCACCACCACCACCCCTGCCAAC | |
| | sProThrProIleProThrProProProProProProLeuProTh | |
| 1261 | AGAGCTCAGAACACCTCTACCACCTACAACCCCAGAAAGGCCAAC | |
| | rGluLeuArgThrProLeuProProThrThrProGluArgProTh | |
| 1306 | CACCGGACTGACAACTATAGCACCAGCTGCCAGTACACCTCCAGG | |
| | rThrGlyLeuThrThrIleAlaProAlaAlaSerThrProProGl | |
| 1351 | AGGGATTACAGTTGACAACAGGGTACAGACAGACCCTCAGAAACC | |
| | yGlyIleThrValAspAsnArgValGlnThrAspProGlnLysPr | |
| 1396 | CAGAGGAGATGTGTTCATTCCACGGCAACCTTCAAATGACTTGTT | |
| | oArgGlyAspValPheIleProArgGlnProSerAsnAspLeuPh | |
| 1441 | TGAAATATTTGAAATAGAAAGAGGAGTCAGTGCAGACGATGAAGC | |
| | eGluIlePheGluIleGluArgGlyValSerAlaAspAspGluAl | |
| 1486 | AAAGGATGATCCAGGTGTTCTGGTACACAGTTGTAATTTTGACCA | |
| | aLysAspAspProGlyValLeuValHisSerCysAsnPheAspHi | |
| 1531 | TGGACTTTGTGGATGGATCAGGGAGAAAGACAATGACTTGCACTG | |
| | sGlyLeuCysGlyTrpIleArgGluLysAspAsnAspLeuHisTr | |
| 1576 | GGAACCAATCAGGGACCCAGCAGGTGGACAATATCTGACAGTGTC | |
| | pGluProIleArgAspProAlaGlyGlyGlnTyrLeuThrValSe | |
| 1621 | GGCAGCCAAAGCCCCAGGGGGAAAAGCTGCACGCTTGGTGCTACC | |
| | rAlaAlaLysAlaProGlyGlyLysAlaAlaArgLeuValLeuPr | |
| 1666 | TCTCGGCCGCCTTATGCATTCAGGGGACCTGTGCCTGTCATTCAG | |
| | oLeuGlyArgLeuMetHisSerGlyAspLeuCysLeuSerPheAr | |
| 1711 | GCACAAGGTGACGGGGCTGCACTCTGGCACACTCCAGGTGTTTGT | |
| | gHisLysValThrGlyLeuHisSerGlyThrLeuGlnValPheVa | |
| 1756 | GAGAAAACACGGTGCCCACGGAGCAGCCCTGTGGGGAAGAAATGG | |
| | lArgLysHisGlyAlaHisGlyAlaAlaLeuTrpGlyArgAsnGl | |
| 1801 | TGGCCATGGCTGGAGGCAAACACAGATCACCTTGCGAGGGGCTGA | |
| | yGlyHisGlyTrpArgGlnThrGlnIleThrLeuArgGlyAlaAs | |
| 1846 | CATCAAGAGCGTCGTCTTCAAAGGTGAAAAAAGGCGTGGTCACAC | |
| | pIleLysSerValValPheLysGlyGluLysArgArgGlyHisTh | |
| 1891 | TGGGGAGATTGGATTAGATGATGTGAGCTTGAAAAAAGGCCACTG | |
| | rGlyGluIleGlyLeuAspAspValSerLeuLysLysGlyHisCy | |
| 1936 | CTCTGAAGAACGCTAACAACTCCAGAACTAACAATGAACTCCTAT | |
| | sSerGluGluArg | (SEQ ID NO:34) |
| 1981 | GTTGCTCTATCCTCTTTTTCCAATTCTCATCTTCTCTCCTCTTCT | |
| 2026 | CCCTTTTATCAGGCCTAGGAGAAGAGTGGGTCAGTGGGTCAGAAG | |
| 2071 | GAAGTCTATTTGGTGACCCAGGTTCTTCTGGCCTGCTTTTGT | (SEQ ID NO:33) |

Analysis of the sequence databases using the BLASTP and BLASTX computer programs revealed that the protein encoded by Clone 16467945.0.88 (PROX 17) has 326 of 332 residues (98%) identical to, and 327 of 332 residues (98%) positive with, the 509 residue human PRO334 protein (ACC: Y13397). In addition, the encoded protein was also found to have 326 of 332 residues (98%) identical to, and 327 of 332 residues (98%) positive with, the 1221 residues mouse protein fibulin-2 (ACC: AAD34456). Furthermore, the encoded protein also has approximately 60% identity, and is approximately 80% positive with, the human 553 residue epidermal growth factor repeat-containing protein (TREMBLNEW-ACC:AAF27812, made public after the filing date of the present invention).

The proteins of the invention encoded by Clone 16467945.0.85 (PROX 16) and Clone 16467945.0.88 (PROX 17) include the proteins disclosed as being encoded by the ORFs described herein, as well as any mature protein arising therefrom as a result of post-translational modifications. Thus, the proteins of the invention encompass both a precursor and any active forms of the 16467945.0.85 and 16467945.0.88 proteins.

Experimental results presented in Example 16 show that, relative to cells from normal tissues, the proteins encoded by Clone 16467945.0.85 (PROX 16) and Clone 16467945.0.88 (PROX 17) are highly over-expressed in certain breast cancer cell lines, ovarian cancer cell lines, renal cancer cell lines, and colon cancer cell lines. In addition, the encoded proteins are strongly suppressed in lung cancer cell lines in comparison with normal lung cells. These results suggest that this clone may be used as a selective probe for detection or diagnosis of these cancers, and that the clones or their gene products may be useful therapeutics or targets in treatment of such cancers.

### PROX Nucleic Acids

The novel nucleic acids of the invention include those that encode a PROX or PROX-like protein, or biologically-active portions thereof. The nucleic acids include nucleic acids encoding polypeptides that include the amino acid sequence of one or more of SEQ ID NO:2n (wherein n=1 to 17). The encoded polypeptides can thus include, e.g., the amino acid sequences of SEQ ID NO:2, 4, 6, 8, 10, 12, 14, 16, 18, 20, 22, 24, 26, 28, 30, 32, and/or 34.

In some embodiments, a nucleic acid encoding a polypeptide having the amino acid sequence of one or more of SEQ ID NO:2n (wherein n=1 to 17) includes the nucleic acid sequence of any of SEQ ID NO:2n-1 (wherein n=1 to 17), or a fragment thereof, and can thus include, e.g., the nucleic acid sequences of SEQ ID NO:1, 3, 5, 7, 9, 11, 13, 15, 17, 19, 21, 23, 25, 27, 29, 31, and/or 33. Additionally, the invention includes mutant or variant nucleic acids of any of SEQ ID NO:2n-1 (wherein n=1 to 17), or a fragment thereof, any of whose bases may be changed from the disclosed sequence while still encoding a protein that maintains its PROX-like biological activities and physiological functions. The invention further includes the complement of the nucleic acid sequence of any of SEQ ID NO:2n-1 (wherein n=1 to 17), including fragments, derivatives, analogs and homologs thereof. The invention additionally includes nucleic acids or nucleic acid fragments, or complements thereto, whose structures include chemical modifications.

Also included are nucleic acid fragments sufficient for use as hybridization probes to identify PROX-encoding nucleic acids (e.g., PROX mRNA) and fragments for use as polymerase chain reaction (PCR) primers for the amplification or mutation of PROX nucleic acid molecules. As used herein, the term “nucleic acid molecule” is intended to include DNA molecules (e.g, cDNA or genomic DNA), RNA molecules (e.g., mRNA), analogs of the DNA or RNA generated using nucleotide analogs, and derivatives, fragments, and homologs thereof. The nucleic acid molecule can be single-stranded or double-stranded, but preferably is double-stranded DNA.

As utilized herein, the term “probes” refer to nucleic acid sequences of variable length, preferably between at least about 10 nucleotides (nt), 100 nt, or as many as about, e.g., 6,000 nt, depending upon the specific use. Probes are used in the detection of identical, similar, or complementary nucleic acid sequences. Longer length probes are usually obtained from a natural or recombinant source, are highly specific and much slower to hybridize than oligomers. Probes may be single- or double-stranded, and may also be designed to have specificity in PCR, membrane-based hybridization technologies, or ELISA-like technologies.

As utilized herein, the term “isolated” nucleic acid molecule is a nucleic acid that is separated from other nucleic acid molecules that are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated PROX nucleic acid molecule can contain less than approximately 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

As used herein, the term a “mature” form of a polypeptide or protein is the product of a naturally occurring polypeptide or precursor form or PROX-protein. The naturally occurring polypeptide, precursor or PROX-protein includes, by way of non-limiting example, the full length gene product, encoded by the corresponding gene. Alternatively, it may be defined as the polypeptide, precursor or PROX-protein encoded by an open reading frame described herein. The product “mature” form arises, again by way of non-limiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristylation, or phosphorylation. In general, a mature polypeptide or protein may result from the operation of only one of these processes, or a combination of any of them.

A nucleic acid molecule of the invention, e.g., a nucleic acid molecule having the nucleotide sequence of SEQ ID NO:2n-1 (wherein n=1 to 17), or a complement of any of these nucleotide sequences, can be isolated using standard molecular biology techniques and the sequence information provided herein. Using all or a portion of the nucleic acid sequence of any of SEQ ID NO:2n-1 (wherein n=1 to 17) as a hybridization probe, PROX nucleic acid sequences can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to PROX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment, an oligonucleotide comprising a nucleic acid molecule less than 100 nt in length would further comprise at lease 6 contiguous nucleotides of any of SEQ ID NO:2n-1 (wherein n=1 to 17), or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in any of SEQ ID NO:2n-1 (wherein n=1 to 17). In still another embodiment, an isolated nucleic acid molecule of the invention comprises a nucleic acid molecule that is a complement of the nucleotide sequence shown in any of SEQ ID NO:2n-1 (wherein n=1 to 17), or a portion of this nucleotide sequence. A nucleic acid molecule that is complementary to the nucleotide sequence shown in is one that is sufficiently complementary to the nucleotide sequence shown in of any of SEQ ID NO:2n-1 (wherein n=1 to 17) that it can hydrogen bond with little or no mismatches to the nucleotide sequence shown in of any of SEQ ID NO:2n-1 (wherein n=1 to 17), thereby forming a stable duplex.

As used herein, the term “complementary” refers to Watson-Crick or Hoogsteen base-pairing between nucleotides units of a nucleic acid molecule, whereas the term “binding” is defined as the physical or chemical interaction between two polypeptides or compounds or associated polypeptides or compounds or combinations thereof. Binding includes ionic, non-ionic, Von der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Additionally, the nucleic acid molecule of the invention can comprise only a portion of the nucleic acid sequence of any of SEQ ID NO:2n-1 (wherein n=1 to 17), e.g., a fragment that can be used as a probe or primer, or a fragment encoding a biologically active portion of PRO. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild-type.

Derivatives and analogs may be full-length or other than full-length, if the derivative or analog contains a modified nucleic acid or amino acid, as described infra. Derivatives or analogs of the nucleic acids or proteins of the invention include, but are not limited to, molecules comprising regions that are substantially homologous to the nucleic acids or proteins of the invention, in various embodiments, by at least about 70%, 80%, 85%, 90%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489), which is incorporated herein by reference in its entirety.

As utilized herein, the term “homologous nucleic acid sequence” or “homologous amino acid sequence,” or variations thereof, refer to sequences characterized by a homology at the nucleotide level or amino acid level as discussed supra. Homologous nucleotide sequences encode those sequences coding for isoforms of PROX polypeptide. Isoforms can be expressed in different tissues of the same organism as a result of, e.g., alternative splicing of RNA. Alternatively, isoforms can be encoded by different genes. In the invention, homologous nucleotide sequences include nucleotide sequences encoding for a PROX polypeptide of species other than humans, including, but not limited to, mammals, and thus can include, e.g., mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the nucleotide sequence encoding human PROX protein. Homologous nucleic acid sequences include those nucleic acid sequences that encode conservative amino acid substitutions (see below) in any of SEQ ID NO:2n (wherein n=1 to 17) as well as a polypeptide having PROX activity. Biological activities of the PROX proteins are described below. A homologous amino acid sequence does not encode the amino acid sequence of a human PROX polypeptide.

The nucleotide sequence determined from the cloning of the human PROX gene allows for the generation of probes and primers designed for use in identifying the cell types disclosed and/or cloning PROX homologues in other cell types, e.g., from other tissues, as well as PROX homologues from other mammals. The probe/primer typically comprises a substantially-purified oligonucleotide. The oligonucleotide typically comprises a region of nucleotide sequence that hybridizes under stringent conditions to at least about 12, 25, 50, 100, 150, 200, 250, 300, 350 or 400 or more consecutive sense strand nucleotide sequence of SEQ ID NO:2n-1 (wherein n=1 to 17); or an anti-sense strand nucleotide sequence of SEQ ID NO:2n-1 (wherein n=1 to 17); or of a naturally occurring mutant of SEQ ID NO:2n-1 (wherein n=1 to 17).

Probes based upon the human PROX nucleotide sequence can be used to detect transcripts or genomic sequences encoding the same or homologous proteins. In various embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which mis-express a PROX protein, such as by measuring a level of a PROX-encoding nucleic acid in a sample of cells from a subject e.g., detecting PROX mRNA levels or determining whether a genomic PROX gene has been mutated or deleted.

As utilized herein, the term “a polypeptide having a biologically-active portion of PRO” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of PRO” can be prepared by isolating a portion of SEQ ID NO:2n-1 (wherein n=1 to 17), that encodes a polypeptide having a PROX biological activity , expressing the encoded portion of PROX protein (e.g., by recombinant expression in vitro), and assessing the activity of the encoded portion of PRO.

### PROX Variants

The invention further encompasses nucleic acid molecules that differ from the disclosed PROX nucleotide sequences due to degeneracy of the genetic code. These nucleic acids therefore encode the same PROX protein as those encoded by the nucleotide sequence shown in SEQ ID NO:2n-1 (wherein n=1 to 17). In another embodiment, an isolated nucleic acid molecule of the invention has a nucleotide sequence encoding a protein having an amino acid sequence shown in any of SEQ ID NO:2n (wherein n=1 to 17).

In addition to the human PROX nucleotide sequence shown in any of SEQ ID NO:2n-1 (wherein n=1 to 17), it will be appreciated by those skilled in the art that DNA sequence polymorphisms that lead to changes in the amino acid sequences of PROX may exist within a population (e.g., the human population). Such genetic polymorphism in the PROX gene may exist among individuals within a population due to natural allelic variation. As used herein, the terms “gene” and “recombinant gene” refer to nucleic acid molecules comprising an open reading frame encoding a PROX protein, preferably a mammalian PROX protein. Such natural allelic variations can typically result in 1-5% variance in the nucleotide sequence of the PROX gene. Any and all such nucleotide variations and resulting amino acid polymorphisms in PROX that are the result of natural allelic variation and that do not alter the functional activity of PROX are intended to be within the scope of the invention.

Additionally, nucleic acid molecules encoding PROX proteins from other species, and thus that have a nucleotide sequence that differs from the human sequence of any of SEQ ID NO:2n-1 (wherein n=1 to 17), are intended to be within the scope of the invention. Nucleic acid molecules corresponding to natural allelic variants and homologues of the PROX cDNAs of the invention can be isolated based on their homology to the human PROX nucleic acids disclosed herein using the human cDNAs, or a portion thereof, as a hybridization probe according to standard hybridization techniques under stringent hybridization conditions.

In another embodiment, an isolated nucleic acid molecule of the invention is at least 6 nucleotides in length and hybridizes under stringent conditions to the nucleic acid molecule comprising the nucleotide sequence of any of SEQ ID NO:2n-1 (wherein n=1 to 17). In another embodiment, the nucleic acid is at least 10, 25, 50, 100, 250, 500 or 750 nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding PROX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tₘ) for the specific sequence at a defined ionic strength and pH. The Tₘ is the temperature (under defined ionic strength, pH and nucleic acid concentration) at which 50% of the probes complementary to the target sequence hybridize to the target sequence at equilibrium. Since the target sequences are generally present at excess, at Tₘ, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. An isolated nucleic acid molecule of the invention that hybridizes under stringent conditions to the sequence of any of SEQ ID NO:2n-1 (wherein n=1 to 17) corresponds to a naturally occurring nucleic acid molecule. As used herein, a “naturally-occurring” nucleic acid molecule refers to an RNA or DNA molecule having a nucleotide sequence that occurs in nature (e g, encodes a natural protein).

In a second embodiment, a nucleic acid sequence that is hybridizable to the nucleic acid molecule comprising the nucleotide sequence of any of SEQ ID NO:2n-1 (wherein n=1 to 17), or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well known in the art. See, e.g, Ausubel et al. (eds.), 1993, CURRENT PROTOCOLS IN MOLECULAR BIOLOGY, John Wiley & Sons, NY, and Kriegler, 1990. GENE TRANSFER AND EXPRESSION, A LABORATORY MANUAL, Stockton Press, NY.

In a third embodiment, a nucleic acid that is hybridizable to the nucleic acid molecule comprising the nucleotide sequence of any of SEQ ID NO:2n-1 (wherein n=1 to 17), or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al., (eds.), 1993. CURRENT PROTOCOLS IN MOLECULAR BIOLOGY, John Wiley & Sons, NY, and Kxiegler, 1990. GENE TRANSFER AND EXPRESSION, A LABORATORY MANUAL, Stockton Press, NY; Shilo and Weinberg, 1981. Proc. Natl. Acad. Sci USA 78: 6789-6792.

### Conservative Mutations

In addition to naturally-occurring allelic variants of the PROX sequence that may exist in the population, the skilled artisan will further appreciate that changes can be introduced by mutation into the nucleotide sequence of any of SEQ ID NO:2n-1 (wherein n=1 to 17), thereby leading to changes in the amino acid sequence of the encoded PROX protein, without altering the functional ability of the PROX protein. For example, nucleotide substitutions leading to amino acid substitutions at “non-essential” amino acid residues can be made in the sequence of any of SEQ ID NO:2n-1 (wherein n=1 to 17). A “non-essential” amino acid residue is a residue that can be altered from the wild-type sequence of PROX without altering the biological activity, whereas an “essential” amino acid residue is required for biological activity. For example, amino acid residues that are conserved among the PROX proteins of the invention, are predicted to be particularly non-amenable to such alteration.

Amino acid residues that are conserved among members of a PROX family members are predicted to be less amenable to alteration. For example, a PROX protein according to the invention can contain at least one domain that is a typically conserved region in a PROX family member. As such, these conserved domains are not likely to be amenable to mutation. Other amino acid residues, however, (e g., those that are not conserved or only semi-conserved among members of the PROX family) may not be as essential for activity and thus are more likely to be amenable to alteration.

Another aspect of the invention pertains to nucleic acid molecules encoding PROX proteins that contain changes in amino acid residues that are not essential for activity. Such PROX proteins differ in amino acid sequence from any of any of SEQ ID NO:2n (wherein n=1 to 17), yet retain biological activity. In one embodiment, the isolated nucleic acid molecule comprises a nucleotide sequence encoding a protein, wherein the protein comprises an amino acid sequence at least about 75% homologous to the amino acid sequence of any of SEQ ID NO:2n (wherein n=1 to 17). Preferably, the protein encoded by the nucleic acid is at least about 80% homologous to any of SEQ ID NO:2n (wherein n=1 to 17), more preferably at least about 90%, 95%, 98%, and most preferably at least about 99% homologous to SEQ ID NO:2n (wherein n=1 to 17).

An isolated nucleic acid molecule encoding a PROX protein homologous to the protein of any of SEQ ID NO:2n (wherein n=1 to 17) can be created by introducing one or more nucleotide substitutions, additions or deletions into the corresponding nucleotide sequence (i.e., SEQ ID NO:2n-1 for the corresponding n), such that one or more amino acid substitutions, additions or deletions are introduced into the encoded protein.

Mutations can be introduced into SEQ ID NO:2n-1 (wherein n=1 to 17) by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Thus, a predicted nonessential amino acid residue in PROX is replaced with another amino acid residue from the same side chain family. Alternatively, in another embodiment, mutations can be introduced randomly along all or part of a PROX coding sequence, such as by saturation mutagenesis, and the resultant mutants can be screened for PROX biological activity to identify mutants that retain activity. Following mutagenesis of SEQ ID NO:2n-1 (wherein n=1 to 17). the encoded protein can be expressed by any recombinant technology known in the art and the activity of the protein can be determined.

In one embodiment, a mutant PROX protein can be assayed for: (i) the ability to form protein:protein interactions with other PROX proteins, other cell-surface proteins, or biologically-active portions thereof; (ii) complex formation between a mutant PROX protein and a PROX receptor; (iii) the ability of a mutant PROX protein to bind to an intracellular target protein or biologically active portion thereof; (e.g., avidin proteins); (iv) the ability to bind BRA protein; or (v) the ability to specifically bind an anti-PROX protein antibody.

### Antisense Nucleic Acids

Another aspect of the invention pertains to isolated antisense nucleic acid molecules that are hybridizable to or complementary to the nucleic acid molecule comprising the nucleotide sequence of SEQ ID NO:2n-1 (wherein n=1 to 17), or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire PROX coding strand, or to only a portion thereof. Nucleic acid molecules encoding fragments, homologs, derivatives and analogs of a PROX protein of any of SEQ ID NO:2n (wherein n=1 to 17) or antisense nucleic acids complementary to a PROX nucleic acid sequence of SEQ ID NO:2n-1 (wherein n=1 to 17) are additionally provided.

In one embodiment, an antisense nucleic acid molecule is antisense to a “coding region” of the coding strand of a nucleotide sequence encoding PRO. The term “coding region” refers to the region of the nucleotide sequence comprising codons which are translated into amino acid residues (eg., the protein coding region of a human PROX that corresponds to any of SEQ ID NO:2n (wherein n=1 to 17)). In another embodiment, the antisense nucleic acid molecule is antisense to a “non-coding region” of the coding strand of a nucleotide sequence encoding PRO. The term “non-coding region” refers to 5′ and 3′ sequences which flank the coding region that are not translated into amino acids (i e., also referred to as 5′ and 3′ non-translated regions).

Given the coding strand sequences encoding PROX disclosed herein (e.g., SEQ ID NO:2n-1 (wherein n=1 to 17) ), antisense nucleic acids of the invention can be designed according to the rules of Watson and Crick or Hoogsteen base-pairing. The antisense nucleic acid molecule can be complementary to the entire coding region of PROX mRNA, but more preferably is an oligonucleotide that is antisense to only a portion of the coding or non-coding region of PROX mRNA. For example, the antisense oligonucleotide can be complementary to the region surrounding the translation start site of PROX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids, e.g, phosphorothioate derivatives and acridine-substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a PROX protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual α-units, the strands run parallel to each other (Gaultier, et al., 1987. Nucl. Acids Res. 15: 6625-6641). The antisense nucleic acid molecule can also comprise a 2′-o-methylribonucleotide (Inoue, et al., 1987. Nucl. Acids Res. 15: 6131-6148) or a chimeric RNA-DNA analogue (Inoue, et al., 1987. FEBS Lett. 215: 327-330).

### Ribozymes and PNA Moieties

Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In still another embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes; described by Haselhoff and Gerlach, 1988. Nature 334: 585-591) can be used to catalytically-cleave PROX mRNA transcripts to thereby inhibit translation of PROX mRNA. A ribozyme having specificity for a PROX-encoding nucleic acid can be designed based upon the nucleotide sequence of a PROX DNA disclosed herein (i.e., SEQ ID NO:2n-1 (wherein n=1 to 17)). For example, a derivative of a Tetrahymena L-19 IVS RNA can be constructed in which the nucleotide sequence of the active site is complementary to the nucleotide sequence to be cleaved in a PROX-encoding mRNA. See, e.g., Cech, et al., U.S. Pat. No. 4,987,071; and Cech, et al., U.S. Pat. No. 5,116,742. Alternatively, PROX mRNA can be used to select a catalytic RNA having a specific ribonuclease activity from a pool of RNA molecules (Bartel, et al., 1993. Science 261: 1411-1418).

Alternatively, PROX gene expression can be inhibited by targeting nucleotide sequences complementary to the regulatory region of the PROX (e.g., the PROX promoter and/or enhancers) to form triple helical structures that prevent transcription of the PROX gene in target cells. See, e.g., Helene, 1991. Anticancer Drug Des. 6: 569-84; Helene, et al., 1992. Ann. N.Y. Acad Sci. 660: 27-36; and Maher, 1992. Bioassays 14: 807-15.

In various embodiments, the nucleic acids of PROX can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (Hyrup, et al., 1996. Bioorg Med. Chem. 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of PROX can be used in therapeutic and diagnostic applications. For example, PNAs can be used as antisense or antigene agents for sequence-specific modulation of gene expression by, e.g, inducing transcription or translation arrest or inhibiting replication. PNAs of PROX can also be used, e.g., in the analysis of single base pair mutations in a gene by, e.g., PNA directed PCR clamping; as artificial restriction enzymes when used in combination with other enzymes, e.g., S1 nucleases (see, Hyrup, 1996., supra); or as probes or primers for DNA sequence and hybridization (see, Hyrup, et al., 1996.; Perry-O'Keefe, 1996., supra).

In another embodiment, PNAs of PROX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of PROX can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, 1996., supra). The synthesis of PNA-DNA chimeras can be performed as described in Finn, et al., (1996. Nucl. Acids Res. 24: 3357-3363). For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Mag, et al., 1989. Nucl. Acid Res. 17: 5973-5988). PNA monomers are then coupled in a stepwise manner to produce a chimeric molecule with a 5′ PNA segment and a 3′ DNA segment (see, Finn, et al., 1996., supra). Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO 88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

### Characterization of PROX Polypeptides

A polypeptide according to the invention includes a polypeptide including the amino acid sequence of PROX polypeptides whose sequences are provided in any SEQ ID NO:2n (wherein n=1 to 17) and includes SEQ ID NOS:2, 4, 6, 8, 10, 12, 14, 16, 18, 20, 22, 24, 26, 28, 30, 32, and/or 34. The invention also includes a mutant or variant protein any of whose residues may be changed from the corresponding residues shown in SEQ ID NOS:2, 4, 6, 8, 10, 12, 14, 16, 18, 20, 22, 24, 26, 28, 30, 32, and/or 34, while still encoding a protein that maintains its PROX activities and physiological functions, or a functional fragment thereof.

In general, a PROX variant that preserves PROX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated PROX proteins, and biologically-active portions thereof, or derivatives, fragments, analogs or homologs thereof. Also provided are polypeptide fragments suitable for use as immunogens to raise anti-PROX antibodies. In one embodiment, native PROX proteins can be isolated from cells or tissue sources by an appropriate purification scheme using standard protein purification techniques. In another embodiment, PROX proteins are produced by recombinant DNA techniques. Alternative to recombinant expression, a PROX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the PROX protein is derived, or substantially free from chemical precursors or other chemicals when chemically synthesized. The language “substantially free of cellular material” includes preparations of PROX proteins in which the protein is separated from cellular components of the cells from which it is isolated or recombinantly-produced. In one embodiment, the language “substantially free of cellular material” includes preparations of PROX proteins having less than about 30% (by dry weight) of non-PROX proteins (also referred to herein as a “contaminating protein”) , more preferably less than about 20% of non-PROX proteins, still more preferably less than about 10% of non-PROX proteins, and most preferably less than about 5% of non-PROX proteins. When the PROX protein or biologically-active portion thereof is recombinantly-produced, it is also preferably substantially free of culture medium, i.e., culture medium represents less than about 20%, more preferably less than about 10%, and most preferably less than about 5% of the volume of the PROX protein preparation.

As utilized herein, the phrase “substantially free of chemical precursors or other chemicals” includes preparations of PROX protein in which the protein is separated from chemical precursors or other chemicals that are involved in the synthesis of the protein. In one embodiment, the language “substantially free of chemical precursors or other chemicals” includes preparations of PROX protein having less than about 30% (by dry weight) of chemical precursors or non-PROX chemicals, more preferably less than about 20% chemical precursors or non-PROX chemicals, still more preferably less than about 10% chemical precursors or non-PROX chemicals, and most preferably less than about 5% chemical precursors or non-PROX chemicals.

Biologically-active portions of a PROX protein include peptides comprising amino acid sequences sufficiently homologous to or derived from the amino acid sequence of the PROX protein which include fewer amino acids than the full-length PROX proteins, and exhibit at least one activity of a PROX protein. Typically, biologically-active portions comprise a domain or motif with at least one activity of the PROX protein. A biologically-active portion of a PROX protein can be a polypeptide which is, for example, 10, 25, 50, 100 or more amino acids in length.

A biologically-active portion of a PROX protein of the invention may contain at least one of the above-identified conserved domains. Moreover, other biologically active portions, in which other regions of the protein are deleted, can be prepared by recombinant techniques and evaluated for one or more of the functional activities of a native PROX protein.

In an embodiment, the PROX protein has an amino acid sequence shown in any of SEQ ID NO:2n (wherein n=1 to 17). In other embodiments, the PROX protein is substantially homologous to any of SEQ ID NO:2n (wherein n=1 to 17) and retains the functional activity of the protein of any of SEQ ID NO:2n (wherein n=1 to 17), yet differs in amino acid sequence due to natural allelic variation or mutagenesis, as described in detail below. Accordingly, in another embodiment, the PROX protein is a protein that comprises an amino acid sequence at least about 45% homologous, and more preferably about 55, 65, 70, 75, 80, 85, 90, 95, 98 or even 99% homologous to the amino acid sequence of any of SEQ ID NO:2n (wherein n=1 to 17) and retains the functional activity of the PROX proteins of the corresponding polypeptide having the sequence of SEQ ID NO:2n (wherein n=1 to 17).

### Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”) .

The nucleic acid sequence homology may be determined as the degree of identity between two sequences. The homology may be determined using computer programs known in the art, such as GAP software provided in the GCG program package. See, Needleman and Wunsch, 1970. J. Mol. Biol. 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NO:2n-1 (wherein n=1 to 17), e.g., SEQ ID NOS:1, 3, 5, 7, 9, 11, 13, 15, 17, 19, 21, 23, 25, 27, 29, 31, 33, 35, and/or 37.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

### Chimeric and Fusion Proteins

The invention also provides PROX chimeric or fusion proteins. As used herein, a PROX “chimeric protein” or “fusion protein” comprises a PROX polypeptide operatively-linked to a non-PROX polypeptide. An “PROX polypeptide” refers to a polypeptide having an amino acid sequence corresponding to a PROX protein shown in SEQ ID NO:2n (wherein n=1 to 17), [e.g., SEQ ID NO:2, 4, 6, 8, 10, 12, 14, 16, 18, 20, 22, 24, 26, 28, 30, 32, and/or 34], whereas a “non-PROX polypeptide” refers to a polypeptide having an amino acid sequence corresponding to a protein that is not substantially homologous to the PROX protein (e.g., a protein that is different from the PROX protein and that is derived from the same or a different organism). Within a PROX fusion protein the PROX polypeptide can correspond to all or a portion of a PROX protein. In one embodiment, a PROX fusion protein comprises at least one biologically-active portion of a PROX protein. In another embodiment, a PROX fusion protein comprises at least two biologically-active portions of a PROX protein. In yet another embodiment, a PROX fusion protein comprises at least three biologically-active portions of a PROX protein. Within the fusion protein, the term “operatively-linked” is intended to indicate that the PROX polypeptide and the non-PROX polypeptide are fused in-frame with one another. The non-PROX polypeptide can be fused to the amino-terminus or carboxyl-terminus of the PROX polypeptide.

In one embodiment, the fusion protein is a GST-PROX fusion protein in which the PROX sequences are fused to the carboxyl-terminus of the GST (glutathione S-transferase) sequences. Such fusion proteins can facilitate the purification of recombinant PROX polypeptides.

In another embodiment, the fusion protein is a PROX protein containing a heterologous signal sequence at its amino-terminus. In certain host cells (e.g., mammalian host cells), expression and/or secretion of PROX can be increased through use of a heterologous signal sequence.

In yet another embodiment, the fusion protein is a PROX-immunoglobulin fusion protein in which the PROX sequences are fused to sequences derived from a member of the immunoglobulin protein family. The PROX-immunoglobulin fusion proteins of the invention can be incorporated into pharmaceutical compositions and administered to a subject to inhibit an interaction between a PROX ligand and a PROX protein on the surface of a cell, to thereby suppress PROX-mediated signal transduction in vivo. The PROX-immunoglobulin fusion proteins can be used to affect the bioavailability of a PROX cognate ligand. Inhibition of the PROX ligand/PROX interaction may be useful therapeutically for both the treatment of proliferative and differentiative disorders, as well as modulating (e.g., promoting or inhibiting) cell survival. Moreover, the PROX-immunoglobulin fusion proteins of the invention can be used as immunogens to produce anti-PROX antibodies in a subject, to purify PROX ligands, and in screening assays to identify molecules that inhibit the interaction of PROX with a PROX ligand.

A PROX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A PROX-encoding nucleic acid can be cloned into such an expression vector such that the fusion moiety is linked in-frame to the PROX protein.

### PROX Agonists and Antagonists

The invention also pertains to variants of the PROX proteins that function as either PROX agonists (i.e., mimetics) or as PROX antagonists. Variants of the PROX protein can be generated by mutagenesis (e.g., discrete point mutation or truncation of the PROX protein). An agonist of a PROX protein can retain substantially the same, or a subset of, the biological activities of the naturally-occurring form of a PROX protein. An antagonist of a PROX protein can inhibit one or more of the activities of the naturally occurring form of a PROX protein by, for example, competitively binding to a downstream or upstream member of a cellular signaling cascade which includes the PROX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the PROX proteins.

Variants of the PROX proteins that function as either PROX agonists (i.e., mimetics) or as PROX antagonists can be identified by screening combinatorial libraries of mutants (e.g., truncation mutants) of the PROX proteins for PROX protein agonist or antagonist activity. In one embodiment, a variegated library of PROX variants is generated by combinatorial mutagenesis at the nucleic acid level and is encoded by a variegated gene library. A variegated library of PROX variants can be produced by, for example, enzymatically-ligating a mixture of synthetic oligonucleotides into gene sequences such that a degenerate set of potential PROX sequences is expressible as individual polypeptides, or alternatively, as a set of larger fusion proteins (e.g., for phage display) containing the set of PROX sequences therein. There are a variety of methods which can be used to produce libraries of potential PROX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential PROX sequences. Methods for synthesizing degenerate oligonucleotides are well-known within the art. See, e.g., Narang, 1983. Tetrahedron 39: 3; Itakuia, et al., 1984. Annu. Rev. Biochem. 53: 323; Itakura, et al., 1984. Science 198: 1056; Ike, et al., 1983. Nucl. Acids Res. 11: 477.

### Polypeptide Libraries

In addition, libraries of fragments of the PROX protein coding sequences can be used to generate a variegated population of PROX fragments for screening and subsequent selection of variants of a PROX protein. In one embodiment, a library of coding sequence fragments can be generated by treating a double-stranded PCR fragment of a PROX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with SI nuclease, and ligating the resulting fragment library into an expression vector. By this method, expression libraries can be derived which encodes amino-terminal and internal fragments of various sizes of the PROX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of PROX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify PROX variants. See, e.g., Arkin and Yourvan, 1992. Proc. Natl. Acad. Sci. USA 89: 7811-7815; Delgrave, et al., 1993. Protein Engineering 6:327-331.

### Anti-PROX Antibodies

The invention encompasses antibodies and antibody fragments, such as F_{ab} or (F_{ab})₂. that bind immunospecifically to any of the PROX polypeptides of said invention.

An isolated PROX protein, or a portion or fragment thereof, can be used as an immunogen to generate antibodies that bind to PROX polypeptides using standard techniques for polyclonal and monoclonal antibody preparation. The full-length PROX proteins can be used or, alternatively, the invention provides antigenic peptide fragments of PROX proteins for use as immunogens. The antigenic PROX peptides comprises at least 4 amino acid residues of the amino acid sequence shown in SEQ ID NO:2n (wherein n=1 to 17) and encompasses an epitope of PROX such that an antibody raised against the peptide forms a specific immune complex with PRO. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

In certain embodiments of the invention, at least one epitope encompassed by the antigenic peptide is a region of PROX that is located on the surface of the protein (e.g., a hydrophilic region). As a means for targeting antibody production, hydropathy plots showing regions of hydrophilicity and hydrophobicity may be generated by any method well known in the art, including, for example, the Kyte-Doolittle or the Hopp-Woods methods, either with or without Fourier transformation (see, e.g., Hopp and Woods, 1981. Proc. Nat. Acad Sci. USA 78: 3824-3828; Kyte and Doolittle, 1982. J. Mol. Biol. 157: 105-142, each incorporated herein by reference in their entirety).

As disclosed herein, PROX protein sequences of SEQ ID NO:2n (wherein n=1 to 17), or derivatives, fragments, analogs, or homologs thereof, may be utilized as immunogens in the generation of antibodies that immunospecifically-bind these protein components. The term “antibody” as used herein refers to immunoglobulin molecules and immunologically-active portions of immunoglobulin molecules, i.e., molecules that contain an antigen binding site that specifically-binds (immunoreacts with) an antigen, such as PRO. Such antibodies include, but are not limited to, polyclonal, monoclonal, chimeric, single chain, F_{ab} and _{(F ab ′) 2} fragments, and an F_{ab} expression library. In a specific embodiment, antibodies to human PROX proteins are disclosed. Various procedures known within the art may be used for the production of polyclonal or monoclonal antibodies to a PROX protein sequence of SEQ ID NO:2n (wherein n=1 to 17), or a derivative, fragment, analog, or homolog thereof. Some of these proteins are discussed, infra.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly-expressed PROX protein or a chemically-synthesized PROX polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against PROX can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of PRO. A monoclonal antibody composition thus typically displays a single binding affinity for a particular PROX protein with which it immunoreacts. For preparation of monoclonal antibodies directed towards a particular PROX protein, or derivatives, fragments, analogs or homologs thereof, any technique that provides for the production of antibody molecules by continuous cell line culture may be utilized. Such techniques include, but are not limited to, the hybridoma technique (see, e.g., Kohler & Milstein, 1975. Nature 256: 495-497); the trioma technique; the human B-cell hybridoma technique (see, e.g., Kozbor, et al., 1983. Immunol. Today 4: 72) and the EBV hybridoma technique to produce human monoclonal antibodies (see, e.g., Cole, et al., 1985. In: MONOCLONAL ANTIBODIES AND CANCER THERAPY, Alan R. Liss, Inc., pp. 77-96). Human monoclonal antibodies may be utilized in the practice of the invention and may be produced by using human hybridomas (see, e.g., Cote, et al., 1983. Proc. Natl. Acad. Sci. USA 80: 2026-2030) or by transforming human B-cells with Epstein Barr Virus in vitro (see, e.g., Cole, et al., 1985. In: MONOCLONAL ANTIBODIES AND CANCER THERAPY, Alan R. Liss, Inc., pp. 77-96). Each of the above citations is incorporated herein by reference in their entirety.

According to the invention, techniques can be adapted for the production of single-chain antibodies specific to a PROX protein (see, e.g., U.S. Pat. No. 4,946,778). In addition, methods can be adapted for the construction of F_{ab} expression libraries (see, e.g., Huse, et al., 1989. Science 246: 1275-1281) to allow rapid and effective identification of monoclonal F_{ab} fragments with the desired specificity for a PROX protein or derivatives, fragments, analogs or homologs thereof. Non-human antibodies can be “humanized” by techniques well known in the art. See, e.g., U.S. Pat. No. 5,225,539. Antibody fragments that contain the idiotypes to a PROX protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Additionally, recombinant anti-PROX antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. Nos. 4,816,567; 5,225,539; European Patent Application No. 125,023; Better, et al., 1988. Science 240: 1041-1043; Liu, et al., 1987. Proc. Natl. Acad. Sci. USA 84: 3439-3443; Liu, et al., 1987. J. Immunol. 139: 3521-3526; Sun, et al., 1987. Proc. Natl. Acad. Sci USA 84: 214-218; Nishimura, et al., 1987. Cancer Res. 47: 999-1005; Wood, et al., 1985. Nature 314 :446-449; Shaw, et al., 1988. J. Natl. Cancer Inst. 80: 1553-1559); Morrison (1985) Science 229:1202-1207; Oi, et al. (1986) BioTechniques 4:214; Jones, et al., 1986. Nature 321: 552-525; Verhoeyan, et al., 1988. Science 239: 1534; and Beidler, et al., 1988. J. Immunol. 141: 4053-4060. Each of the above citations are incorporated herein by reference in their entirety.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of a PROX protein is facilitated by generation of hybridomas that bind to the fragment of a PROX protein possessing such a domain. Thus, antibodies that are specific for a desired domain within a PROX protein, or derivatives, fragments, analogs or homologs thereof, are also provided herein.

Anti-PROX antibodies may be used in methods known within the art relating to the localization and/or quantitation of a PROX protein (e.g., for use in measuring levels of the PROX protein within appropriate physiological samples, for use in diagnostic methods, for use in imaging the protein, and the like). In a given embodiment, antibodies for PROX proteins, or derivatives, fragments, analogs or homologs thereof, that contain the antibody derived binding domain, are utilized as pharmacologically-active compounds (hereinafter “Therapeutics”).

An anti-PROX antibody (e.g., monoclonal antibody) can be used to isolate a PROX polypeptide by standard techniques, such as affinity chromatography or immunoprecipitation. An anti-PROX antibody can facilitate the purification of natural PROX polypeptide from cells and of recombinantly-produced PROX polypeptide expressed in host cells. Moreover, an anti-PROX antibody can be used to detect PROX protein (e.g., in a cellular lysate or cell supernatant) in order to evaluate the abundance and pattern of expression of the PROX protein. Anti-PROX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidinfbiotin and avidinlbiotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

### PROX Recombinant Expression Vectors and Host Cells

Another aspect of the invention pertains to vectors, preferably expression vectors, containing a nucleic acid encoding a PROX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present Specification, “plasmid” and “vector” can be used interchangeably, as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

As utilized herein, the phrase “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., PROX proteins, mutant forms of PROX proteins, fusion proteins, etc.).

The recombinant expression vectors of the invention can be designed for expression of PROX proteins in prokaryotic or eukaryotic cells. For example, PROX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T₇ promoter regulatory sequences and T₇ polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xₐ, thrombin, and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion Escherichia coli expression vectors include pTrc (Amrann et al., (1988) Gene 69:301-315) and pET 11d (Studier, et al., GENE EXPRESSION TECHNOLOGY: METHODS IN ENZYMOLOGY 185, Academic Press, San Diego, Calif. (1990) 60-89).

One strategy to maximize recombinant protein expression in Escherichia coli is to express the protein in a host bacteria with an impaired capacity to proteolytically-cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in Escherichia coli (see, e.g., Wada, et al., 1992. Nucl. Acids Res. 20: 2111-2118). Such alteration of nucleic acid sequences of the invention can be carried out by standard DNA synthesis techniques.

In another embodiment, the PROX expression vector is a yeast expression vector. Examples of vectors for expression in yeast Saccharomyces cerivisae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, PROX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; see, Pinkert, et al., 1987. Genes Dev. 1: 268-277), lymphoid-specific promoters (see, Calame and Eaton, 1988. Adv. Immunol. 43: 235-275), in particular promoters of T cell receptors (see, Winoto and Baltimore, 1989. EMBO J. 8: 729-733) and immunoglobulins (see, Banerji, et al., 1983. Cell 33: 729-740; Queen and Baltimore, 1983. Cell 33: 741-748), neuron-specific promoters (e.g., the neurofilament promoter; see, Byrne and Ruddle, 1989. Proc. Natl. Acad. Sci. USA 86: 5473-5477), pancreas-specific promoters (see, Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (see, Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to PROX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, PROX protein can be expressed in bacterial cells such as Escherichia coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin, and methotrexate. Nucleic acid encoding a selectable marker can be introduced into a host cell on the same vector as that encoding PROX or can be introduced on a separate vector. Cells stably-transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) PROX protein. Accordingly, the invention further provides methods for producing PROX protein using the host cells of the invention. In one embodiment, the method comprises culturing the host cell of invention (i.e., into which a recombinant expression vector encoding PROX protein has been introduced) in a suitable medium such that PROX protein is produced. In another embodiment, the method further comprises isolating PROX protein from the medium or the host cell.

### Transgenic Animals

The host cells of the invention can also be used to produce non-human transgenic animals. For example, in one embodiment, a host cell of the invention is a fertilized oocyte or an embryonic stem cell into which PROX protein-coding sequences have been introduced. These host cells can then be used to create non-human transgenic animals in which exogenous PROX sequences have been introduced into their genome or homologous recombinant animals in which endogenous PROX sequences have been altered. Such animals are useful for studying the function and/or activity of PROX protein and for identifying and/or evaluating modulators of PROX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc.

A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous PROX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing PROX-encoding nucleic acid into the male pronuclei of a fertilized oocyte (e.g., by micro-injection, retroviral infection) and allowing the oocyte to develop in a pseudopregnant female foster animal. The human PROX cDNA sequences of SEQ ID NO:2n-1 (wherein n=1 to 17), can be introduced as a transgene into the genome of a non-human animal. Alternatively, a non-human homologue of the human PROX gene, such as a mouse PROX gene, can be isolated based on hybridization to the human PROX cDNA (described further supra) and used as a transgene. Intronic sequences and polyadenylation signals can also be included in the transgene to increase the efficiency of expression of the transgene. A tissue-specific regulatory sequence(s) can be operably-linked to the PROX transgene to direct expression of PROX protein to particular cells. Methods for generating transgenic animals via embryo manipulation and micro-injection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the PROX transgene in its genome and/or expression of PROX mRNA in tissues or cells of the animals. A transgenic founder animal can then be used to breed additional animals carrying the transgene. Moreover, transgenic animals carrying a transgene-encoding PROX protein can further be bred to other transgenic animals carrying other transgenes.

To create a homologous recombinant animal, a vector is prepared which contains at least a portion of a PROX gene into which a deletion, addition or substitution has been introduced to thereby alter, e.g., functionally disrupt, the PROX gene. The PROX gene can be a human gene (e.g., the cDNA of SEQ ID NO:2n-1 (wherein n=1 to 17)), but more preferably, is a non-human homologue of a human PROX gene. For example, a mouse homologue of human PROX gene of SEQ ID NO:2n-1 (wherein n=1 to 17), can be used to construct a homologous recombination vector suitable for altering an endogenous PROX gene in the mouse genome. In one embodiment, the vector is designed such that, upon homologous recombination, the endogenous PROX gene is functionally disrupted (i e., no longer encodes a functional protein; also referred to as a “knock out” vector).

Alternatively, the vector can be designed such that, upon homologous recombination, the endogenous PROX gene is mutated or otherwise altered but still encodes functional protein (e.g., the upstream regulatory region can be altered to thereby alter the expression of the endogenous PROX protein). In the homologous recombination vector, the altered portion of the PROX gene is flanked at its 5′- and 3′-termini by additional nucleic acid of the PROX gene to allow for homologous recombination to occur between the exogenous PROX gene carried by the vector and an endogenous PROX gene in an embryonic stem cell. The additional flanking PROX nucleic acid is of sufficient length for successful homologous recombination with the endogenous gene. Typically, several kilobases (Kb) of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced PROX gene has homologously-recombined with the endogenous PROX gene are selected. See, e.g., Li, et al., 1992. Cell 69: 915.

The selected cells are then micro-injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-human animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte, and then transferred to pseudopregnant female foster animal. The offspring borne of this female foster animal will be a clone of the animal from which the cell (e.g., the somatic cell) is isolated.

### Pharmaceutical Compositions

The PROX nucleic acid molecules, PROX proteins, and anti-PROX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically-acceptable carrier. As used herein, “pharmaceutically-acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and other non-aqueous (i.e., lipophilic) vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a PROX protein or anti-PROX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

### Screening and Detection Methods

The nucleic acid molecules, proteins, protein homologues, and antibodies described herein can be used in one or more of the following methods: (i) screening assays; (ii) detection assays (e.g., chromosomal mapping, cell and tissue typing, forensic biology), (iii) predictive medicine (e.g., diagnostic assays, prognostic assays, monitoring clinical trials, and pharmacogenomics); and (iv) methods of treatment (e.g., therapeutic and prophylactic).

The isolated nucleic acid molecules of the present invention can be used to express PROX protein (e.g., via a recombinant expression vector in a host cell in gene therapy applications), to detect PROX mRNA (e.g., in a biological sample) or a genetic lesion in an PROX gene, and to modulate PROX activity, as described further, infra. In addition, the PROX proteins can be used to screen drugs or compounds that modulate the PROX protein activity or expression as well as to treat disorders characterized by insufficient or excessive production of PROX protein or production of PROX protein forms that have decreased or aberrant activity compared to PROX wild-type protein. In addition, the anti-PROX antibodies of the present invention can be used to detect and isolate PROX proteins and modulate PROX activity.

The invention further pertains to novel agents identified by the screening assays described herein and uses thereof for treatments as described, supra.

### Screening Assays

The invention provides a method (also referred to herein as a “screening assay”) for identifying modulators, i.e., candidate or test compounds or agents (e.g., peptides, peptidomimetics, small molecules or other drugs) that bind to PROX proteins or have a stimulatory or inhibitory effect on, e.g., PROX protein expression or PROX protein activity. The invention also includes compounds identified in the screening assays described herein.

In one embodiment, the invention provides assays for screening candidate or test compounds which bind to or modulate the activity of the membrane-bound form of a PROX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of PROX protein, or a biologically-active portion thereof, on the cell surface is contacted with a test compound and the ability of the test compound to bind to a PROX protein determined. The cell, for example, can of mammalian origin or a yeast cell. Determining the ability of the test compound to bind to the PROX protein can be accomplished, for example, by coupling the test compound with a radioisotope or enzymatic label such that binding of the test compound to the PROX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of PROX protein, or a biologically-active portion thereof, on the cell surface with a known compound which binds PROX to form an assay mixture, contacting the assay mixture with a test compound, and determining the ability of the test compound to interact with a PROX protein, wherein determining the ability of the test compound to interact with a PROX protein comprises determining the ability of the test compound to preferentially bind to PROX protein or a biologically-active portion thereof as compared to the known compound.

In another embodiment, an assay is a cell-based assay comprising contacting a cell expressing a membrane-bound form of PROX protein, or a biologically-active portion thereof, on the cell surface with a test compound and determining the ability of the test compound to modulate (e.g., stimulate or inhibit) the activity of the PROX protein or biologically-active portion thereof. Determining the ability of the test compound to modulate the activity of PROX or a biologically-active portion thereof can be accomplished, for example, by determining the ability of the PROX protein to bind to or interact with a PROX target molecule. As used herein, a “target molecule” is a molecule with which a PROX protein binds or interacts in nature, for example, a molecule on the surface of a cell which expresses a PROX interacting protein, a molecule on the surface of a second cell, a molecule in the extracellular milieu, a molecule associated with the internal surface of a cell membrane or a cytoplasmic molecule. An PROX target molecule can be a non-PROX molecule or a PROX protein or polypeptide of the invention. In one embodiment, a PROX target molecule is a component of a signal transduction pathway that facilitates transduction of an extracellular signal (e.g. a signal generated by binding of a compound to a membrane-bound PROX molecule) through the cell membrane and into the cell. The target, for example, can be a second intercellular protein that has catalytic activity or a protein that facilitates the association of downstream signaling molecules with PRO.

Determining the ability of the PROX protein to bind to or interact with a PROX target molecule can be accomplished by one of the methods described above for determining direct binding. In one embodiment, determining the ability of the PROX protein to bind to or interact with a PROX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a PROX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a PROX protein or biologically-active portion thereof with a test compound and determining the ability of the test compound to bind to the PROX protein or biologically-active portion thereof. Binding of the test compound to the PROX protein can be determined either directly or indirectly as described above. In one such embodiment, the assay comprises contacting the PROX protein or biologically-active portion thereof with a known compound which binds PROX to form an assay mixture, contacting the assay mixture with a test compound, and determining the ability of the test compound to interact with a PROX protein, wherein determining the ability of the test compound to interact with a PROX protein comprises determining the ability of the test compound to preferentially bind to PROX or biologically-active portion thereof as compared to the known compound.

In still another embodiment, an assay is a cell-free assay comprising contacting PROX protein or biologically-active portion thereof with a test compound and determining the ability of the test compound to modulate (e.g. stimulate or inhibit) the activity of the PROX protein or biologically-active portion thereof. Determining the ability of the test compound to modulate the activity of PROX can be accomplished, for example, by determining the ability of the PROX protein to bind to a PROX target molecule by one of the methods described above for determining direct binding. In an alternative embodiment, determining the ability of the test compound to modulate the activity of PROX protein can be accomplished by determining the ability of the PROX protein further modulate a PROX target molecule. For example, the catalytic/enzymatic activity of the target molecule on an appropriate substrate can be determined as described, supra.

In yet another embodiment, the cell-free assay comprises contacting the PROX protein or biologically-active portion thereof with a known compound which binds PROX protein to form an assay mixture, contacting the assay mixture with a test compound, and determining the ability of the test compound to interact with a PROX protein, wherein determining the ability of the test compound to interact with a PROX protein comprises determining the ability of the PROX protein to preferentially bind to or modulate the activity of a PROX target molecule.

The cell-free assays of the invention are amenable to use of both the soluble form or the membrane-bound form of PROX protein. In the case of cell-free assays comprising the membrane-bound form of PROX protein, it may be desirable to utilize a solubilizing agent such that the membrane-bound form of PROX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton®X-100, Triton®X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either PROX protein or its target molecule to facilitate separation of complexed from uncomplexed forms of one or both of the proteins, as well as to accommodate automation of the assay. Binding of a test compound to PROX protein, or interaction of PROX protein with a target molecule in the presence and absence of a candidate compound, can be accomplished in any vessel suitable for containing the reactants. Examples of such vessels include microtiter plates, test tubes, and micro-centrifuige tubes. In one embodiment, a fusion protein can be provided that adds a domain that allows one or both of the proteins to be bound to a matrix. For example, GST-PROX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or PROX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of PROX protein binding or activity determined using standard techniques.

Other techniques for immobilizing proteins on matrices can also be used in the screening assays of the invention. For example, either the PROX protein or its target molecule can be immobilized utilizing conjugation of biotin and streptavidin. Biotinylated PROX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with PROX protein or target molecules, but which do not interfere with binding of the PROX protein to its target molecule, can be derivatized to the wells of the plate, and unbound target or PROX protein trapped in the wells by antibody conjugation. Methods for detecting such complexes, in addition to those described above for the GST-immobilized complexes, include immunodetection of complexes using antibodies reactive with the PROX protein or target molecule, as well as enzyme-linked assays that rely on detecting an enzymatic activity associated with the PROX protein or target molecule.

In another embodiment, modulators of PROX protein expression are identified in a method wherein a cell is contacted with a candidate compound and the expression of PROX mRNA or protein in the cell is determined. The level of expression of PROX mRNA or protein in the presence of the candidate compound is compared to the level of expression of PROX mRNA or protein in the absence of the candidate compound. The candidate compound can then be identified as a modulator of PROX mRNA or protein expression based upon this comparison. For example, when expression of PROX mRNA or protein is greater (i.e., statistically significantly greater) in the presence of the candidate compound than in its absence, the candidate compound is identified as a stimulator of PROX mRNA or protein expression. Alternatively, when expression of PROX mRNA or protein is less (statistically significantly less) in the presence of the candidate compound than in its absence, the candidate compound is identified as an inhibitor of PROX mRNA or protein expression. The level of PROX mRNA or protein expression in the cells can be determined by methods described herein for detecting PROX mRNA or protein.

In yet another aspect of the invention, the PROX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with PROX (“PROX-binding proteins” or “PROX-bp”) and modulate PROX activity. Such PROX-binding proteins are also likely to be involved in the propagation of signals by the PROX proteins as, for example, upstream or downstream elements of the PROX pathway.

The two-hybrid system is based on the modular nature of most transcription factors, which consist of separable DNA-binding and activation domains. Briefly, the assay utilizes two different DNA constructs. In one construct, the gene that codes for PROX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a PROX-dependent complex, the DNA-binding and activation domains of the transcription factor are brought into close PROX imity. This PROX imity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with PRO.

The invention further pertains to novel agents identified by the aforementioned screening assays and uses thereof for treatments as described herein.

### Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, infra.

### Chromosome Mapping

Once the sequence (or a portion of the sequence) of a gene has been isolated, this sequence can be used to map the location of the gene on a chromosome. This process is called chromosome mapping. Accordingly, portions or fragments of the PROX sequences shown in SEQ ID NO:2n-1 (wherein n=1 to 17), or fragments or derivatives thereof, can be used to map the location of the PROX genes, respectively, on a chromosome. The mapping of the PROX sequences to chromosomes is an important first step in correlating these sequences with genes associated with disease.

Briefly, PROX genes can be mapped to chromosomes by preparing PCR primers (preferably 15-25 bp in length) from the PROX sequences. Computer analysis of the PRO, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the PROX sequences will yield an amplified fragment.

Somatic cell hybrids are prepared by fusing somatic cells from different mammals (eg., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. -Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the PROX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, NY 1988).

Reagents for chromosome mapping can be used individually to mark a single chromosome or a single site on that chromosome, or panels of reagents can be used for marking multiple sites and/or multiple chromosomes. Reagents corresponding to non-coding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Additionally, differences in the DNA sequences between individuals affected and unaffected with a disease associated with the PROX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

### Tissue Typing

The PROX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” as described in U.S. Pat. No. 5,272,057).

Furthermore, the sequences of the invention can be used to provide an alternative technique that determines the actual base-by-base DNA sequence of selected portions of an individual's genome. Thus, the PROX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The PROX sequences of the invention uniquely represent portions of the human genome. Allelic variation occurs to some degree in the coding regions of these sequences, and to a greater degree in the non-coding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the non-coding regions, fewer sequences are necessary to differentiate individuals. The non-coding sequences can comfortably provide positive individual identification with a panel of perhaps 10 to 1,000 primers that each yield a non-coding amplified sequence of 100 bases. If predicted coding sequences, such as those in SEQ ID NO:2n-1 (wherein n=1 to 17) are used, a more appropriate number of primers for positive individual identification would be 500-2,000.

### Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining PROX protein and/or nucleic acid expression as well as PROX activity, in the context of a biological sample (e.g., blood, serum, cells, tissue) to thereby determine whether an individual is afflicted with a disease or disorder, or is at risk of developing a disorder, associated with aberrant PROX expression or activity. The invention also provides for prognostic (or predictive) assays for determining whether an individual is at risk of developing a disorder associated with PROX protein, nucleic acid expression or activity. For example, mutations in a PROX gene can be assayed in a biological sample. Such assays can be used for prognostic or predictive purpose to thereby prophylactically treat an individual prior to the onset of a disorder characterized by or associated with PROX protein, nucleic acid expression or activity.

Another aspect of the invention provides methods for determining PROX protein, nucleic acid expression or PROX activity in an individual to thereby select appropriate therapeutic or prophylactic agents for that individual (referred to herein as “pharmacogenomics”) . Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of PROX in clinical trials.

### Use of Partial PROX Sequences in Forensic Biology

DNA-based identification techniques can also be used in forensic biology. Forensic biology is a scientific field employing genetic typing of biological evidence found at a crime scene as a means for positively identifying, e.g., a perpetrator of a crime. To make such an identification, PCR technology can be used to amplify DNA sequences taken from very small biological samples such as tissues (e.g., hair or skin, or body fluids, e.g., blood, saliva, or semen found at a crime scene). The amplified sequence can then be compared to a standard, thereby allowing identification of the origin of the biological sample.

The sequences of the invention can be used to provide polynucleotide reagents, e.g., PCR primers, targeted to specific loci in the human genome, that can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to non-coding regions of SEQ ID NO:2n-1 (where n=1 to 17) are particularly appropriate for this use as greater numbers of polymorphisms occur in the non-coding regions, making it easier to differentiate individuals using this technique. Examples of polynucleotide reagents include the PROX sequences or portions thereof, e.g., fragments derived from the non-coding regions of one or more of SEQ ID NO:2n-1 (where n=1 to 17), having a length of at least 20 bases, preferably at least 30 bases.

The PROX sequences described herein can further be used to provide polynucleotide reagents, e.g., labeled or label-able probes that can be used, for example, in an in situ hybridization technique, to identify a specific tissue (e.g., brain tissue, etc). This can be very useful in cases where a forensic pathologist is presented with a tissue of unknown origin. Panels of such PROX probes can be used to identify tissue by species and/or by organ type.

In a similar fashion, these reagents, e.g., PROX primers or probes can be used to screen tissue culture for contamination (i.e., screen for the presence of a mixture of different types of cells in a culture).

### Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining PROX protein and/or nucleic acid expression as well as PROX activity, in the context of a biological sample (e.g., blood, serum, cells, tissue) to thereby determine whether an individual is afflicted with a disease or disorder, or is at risk of developing a disorder, associated with aberrant PROX expression or activity. The invention also provides for prognostic (or predictive) assays for determining whether an individual is at risk of developing a disorder associated with PROX protein, nucleic acid expression or activity. For example, mutations in a PROX gene can be assayed in a biological sample. Such assays can be used for prognostic or predictive purpose to thereby prophylactically treat an individual prior to the onset of a disorder characterized by or associated with PROX protein, nucleic acid expression, or biological activity.

Another aspect of the invention provides methods for determining PROX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of PROX in clinical trials. These and other agents are described in further detail in the following sections.

### Diagnostic Assays

An exemplary method for detecting the presence or absence of PROX in a biological sample involves obtaining a biological sample from a test subject and contacting the biological sample with a compound or an agent capable of detecting PROX protein or nucleic acid (e.g., mRNA, genomic DNA) that encodes PROX protein such that the presence of PROX is detected in the biological sample. An agent for detecting PROX mRNA or genomic DNA is a labeled nucleic acid probe capable of hybridizing to PROX mRNA or genomic DNA. The nucleic acid probe can be, for example, a full-length PROX nucleic acid, such as the nucleic acid of SEQ ID NO:2n-1 (wherein n=1 to 17), or a portion thereof, such as an oligonucleotide of at least 15, 30, 50, 100, 250 or 500 nucleotides in length and sufficient to specifically hybridize under stringent conditions to PROX mRNA or genomic DNA. Other suitable probes for use in the diagnostic assays of the invention are described herein.

An agent for detecting PROX protein is an antibody capable of binding to PROX protein, preferably an antibody with a detectable label. Antibodies can be polyclonal, or more preferably, monoclonal. An intact antibody, or a fragment thereof (e.g., F_{ab} or F_{(ab)2}) can be used. As utilized herein, the term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. As utilized herein, the term “biological sample” is intended to include tissues, cells and biological fluids isolated from a subject, as well as tissues, cells and fluids present within a subject. That is, the detection method of the invention can be used to detect PROX mRNA, protein, or genomic DNA in a biological sample in vitro as well as in vivo. For example, in vitro techniques for detection of PROX mRNA include Northern hybridizations and in situ hybridizations. In vitro techniques for detection of PROX protein include enzyme linked immunosorbent assays (ELISAs), Western blots, immunoprecipitations, and immunofluorescence. In vitro techniques for detection of PROX genomic DNA include Southern hybridizations. Furthermore, in vivo techniques for detection of PROX protein include introducing into a subject a labeled anti-PROX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting PROX protein, mRNA, or genomic DNA, such that the presence of PROX protein, mRNA or genomic DNA is detected in the biological sample, and comparing the presence of PROX protein, mRNA or genomic DNA in the control sample with the presence of PROX protein, mRNA or genomic DNA in the test sample.

The invention also encompasses kits for detecting the presence of PROX in a biological sample. For example, the kit can comprise: a labeled compound or agent capable of detecting PROX protein or mRNA in a biological sample; means for determining the amount of PROX in the sample; and means for comparing the amount of PROX in the sample with a standard. The compound or agent can be packaged in a suitable container. The kit can further comprise instructions for using the kit to detect PROX protein or nucleic acid.

### Prognostic Assays

The diagnostic methods described herein can furthermore be utilized to identify subjects having or at risk of developing a disease or disorder associated with aberrant PROX expression or activity. For example, the assays described herein, such as the preceding diagnostic assays or the following assays, can be utilized to identify a subject having or at risk of developing a disorder associated with PROX protein, nucleic acid expression or activity. Alternatively, the prognostic assays can be utilized to identify a subject having or at risk for developing a disease or disorder. Thus, the invention provides a method for identifying a disease or disorder associated with aberrant PROX expression or activity in which a test sample is obtained from a subject and PROX protein or nucleic acid (e.g., mRNA, genomic DNA) is detected, wherein the presence of PROX protein or nucleic acid is diagnostic for a subject having or at risk of developing a disease or disorder associated with aberrant PROX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant PROX expression or activity. For example, such methods can be used to determine whether a subject can be effectively treated with an agent for a disorder. Thus, the invention provides methods for determining whether a subject can be effectively treated with an agent for a disorder associated with aberrant PROX expression or activity in which a test sample is obtained and PROX protein or nucleic acid is detected (e.g., wherein the presence of PROX protein or nucleic acid is diagnostic for a subject that can be administered the agent to treat a disorder associated with aberrant PROX expression or activity).

The methods of the invention can also be used to detect genetic lesions in a PROX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a PROX-protein, or the mis-expression of the PROX gene. For example, such genetic lesions can be detected by ascertaining the existence of at least one of: (i) a deletion of one or more nucleotides from a PROX gene; (ii) an addition of one or more nucleotides to a PROX gene; (iii) a substitution of one or more nucleotides of a PROX gene, (iv) a chromosomal rearrangement of a PROX gene; (v) an alteration in the level of a messenger RNA transcript of a PROX gene; (vi) aberrant modification of a PROX gene, such as of the methylation pattern of the genomic DNA; (vii) the presence of a non-wild-type splicing pattern of a messenger RNA transcript of a PROX gene; (viii) a non-wild-type level of a PROX protein, (ix) allelic loss of a PROX gene; and (x) inappropriate post-translational modification of a PROX protein. As described herein, there are a large number of assay techniques known in the art which can be used for detecting lesions in a PROX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the PROX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a PROX gene under conditions such that hybridization and amplification of the PROX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a PROX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in PROX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in PROX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the PROX gene and detect mutations by comparing the sequence of the sample PROX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. BioTechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the PROX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type PROX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in PROX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a PROX sequence, e.g., a wild-type PROX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in PROX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control PROX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a PROX gene.

Furthermore, any cell type or tissue, preferably peripheral blood leukocytes, in which PROX is expressed may be utilized in the prognostic assays described herein. However, any biological sample containing nucleated cells may be used, including, for example, buccal mucosal cells.

### Pharmacogenomics

Agents, or modulators that have a stimulatory or inhibitory effect on PROX activity (e.g., PROX gene expression), as identified by a screening assay described herein can be administered to individuals to treat (prophylactically or therapeutically) disorders (e.g., cancer or immune disorders associated with aberrant PROX activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of PROX protein, expression of PROX nucleic acid, or mutation content of PROX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol. 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of PROX protein, expression of PROX nucleic acid, or mutation content of PROX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a PROX modulator, such as a modulator identified by one of the exemplary screening assays described herein.

### Monitoring of Effects During Clinical Trials

Monitoring the influence of agents (e.g., drugs, compounds) on the expression or activity of PROX (e.g., the ability to modulate aberrant cell proliferation and/or differentiation) can be applied not only in basic drug screening, but also in clinical trials. For example, the effectiveness of an agent determined by a screening assay as described herein to increase PROX gene expression, protein levels, or upregulate PROX activity, can be monitored in clinical trails of subjects exhibiting decreased PROX gene expression, protein levels, or downregulated PROX activity. Alternatively, the effectiveness of an agent determined by a screening assay to decrease PROX gene expression, protein levels, or downregulate PROX activity, can be monitored in clinical trails of subjects exhibiting increased PROX gene expression, protein levels, or upregulated PROX activity. In such clinical trials, the expression or activity of PROX and, preferably, other genes that have been implicated in, for example, a cellular proliferation or immune disorder can be used as a “read out” or markers of the immune responsiveness of a particular cell.

By way of example, and not of limitation, genes, including PRO, that are modulated in cells by treatment with an agent (e.g., compound, drug or small molecule) that modulates PROX activity (e.g., identified in a screening assay as described herein) can be identified. Thus, to study the effect of agents on cellular proliferation disorders, for example, in a clinical trial, cells can be isolated and RNA prepared and analyzed for the levels of expression of PROX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of PROX or other genes. In this manner, the gene expression pattern can serve as a marker, indicative of the physiological response of the cells to the agent. Accordingly, this response state may be determined before, and at various points during, treatment of the individual with the agent

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a PROX protein, mRNA, or genomic DNA in the pre-administration sample; (iii) obtaining one or more post-administration samples from the subject; (iv) detecting the level of expression or activity of the PROX protein, mRNA, or genomic DNA in the post-administration samples; (v) comparing the level of expression or activity of the PROX protein, mRNA, or genomic DNA in the pre-administration sample with the PROX protein, mRNA, or genomic DNA in the post administration sample or samples; and (vi) altering the administration of the agent to the subject accordingly. For example, increased administration of the agent may be desirable to increase the expression or activity of PROX to higher levels than detected, i.e., to increase the effectiveness of the agent. Alternatively, decreased administration of the agent may be desirable to decrease expression or activity of PROX to lower levels than detected, i.e., to decrease the effectiveness of the agent.

### Methods of Treatment

The invention provides for both prophylactic and therapeutic methods of treating a subject at risk of (or susceptible to) a disorder or having a disorder associated with aberrant PROX expression or activity. These methods of treatment will be discussed more fully, infra.

### Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof, (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endoggenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof, or an agonist that increases bioavailability.

Increased or decreased levels can be readily detected by quantifying peptide and/or RNA, by obtaining a patient tissue sample (e g, from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

### Prophylactic Methods

In one aspect, the invention provides a method for preventing, in a subject, a disease or condition associated with an aberrant PROX expression or activity, by administering to the subject an agent that modulates PROX expression or at least one PROX activity. Subjects at risk for a disease that is caused or contributed to by aberrant PROX expression or activity can be identified by, for example, any or a combination of diagnostic or prognostic assays as described herein. Administration of a prophylactic agent can occur prior to the manifestation of symptoms characteristic of the PROX aberrancy, such that a disease or disorder is prevented or, alternatively, delayed in its progression. Depending upon the type of PROX aberrancy, for example, a PROX agonist or PROX antagonist agent can be used for treating the subject. The appropriate agent can be determined based on screening assays described herein.

### Therapeutic Methods

Another aspect of the invention pertains to methods of modulating PROX expression or activity for therapeutic purposes. The modulatory method of the invention involves contacting a cell with an agent that modulates one or more of the activities of PROX protein activity associated with the cell. An agent that modulates PROX protein activity can be an agent as described herein, such as a nucleic acid or a protein, a naturally-occurring cognate ligand of a PROX protein, a peptide, a PROX peptidomimetic, or other small molecule. In one embodiment, the agent stimulates one or more PROX protein activity. Examples of such stimulatory agents include active PROX protein and a nucleic acid molecule encoding PROX that has been introduced into the cell. In another embodiment, the agent inhibits one or more PROX protein activity. Examples of such inhibitory agents include antisense PROX nucleic acid molecules and anti-PROX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a PROX protein or nucleic acid molecule. In one embodiment, the method involves administering an agent (e.g., an agent identified by a screening assay described herein), or combination of agents that modulates (e.g., up-regulates or down-regulates) PROX expression or activity. In another embodiment, the method involves administering a PROX protein or nucleic acid molecule as therapy to compensate for reduced or aberrant PROX expression or activity

Stimulation of PROX activity is desirable in situations in which PROX is abnormally downregulated and/or in which increased PROX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., pre-clampsia).

### Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

### Prophylactic and Therapeutic Uses of the Compositions of the Invention

The PROX nucleic acids and proteins of the invention may be useful in a variety of potential prophylactic and therapeutic applications. By way of a non-limiting example, a cDNA encoding the PROX protein of the invention may be useful in gene therapy, and the protein may be useful when administered to a subject in need thereof.

Both the novel nucleic acids encoding the PROX proteins, and the PROX proteins of the invention, or fragments thereof, may also be useful in diagnostic applications, wherein the presence or amount of the nucleic acid or the protein are to be assessed. These materials are further useful in the generation of antibodies which immunospecifically-bind to the novel substances of the invention for use in therapeutic or diagnostic methods.

The invention will be further illustrated in the following non-limiting examples.

### EXAMPLE 1

### Mapping the Chromosomal Location of PRO1 and PRO3 Nucleic Acid Sequences

Radiation hybrid mapping, using human chromosome markers, was performed for PRO1 and PRO3 nucleic acid sequences. The procedure used to obtain these results was analogous to that described in Steen, et al., 1999. A High-Density Integrated Genetic Linkage and Radiation Hybrid Map of the Laboratory Rat, Genome Res. (Published Online on May 21, 1999) 9: AP1-AP8. A panel of 93 cell clones containing randomized radiation-induced human chromosomal fragments was screened in 96 well plates using PCR primers designed to identify the sought clones in a unique fashion. Table 19 shows the two markers between which each of two clones of the present invention (i e., Clone 20468752.0.18 (PROX 1) and Clone 11692010.0.51 (PROX 3)) are found, and their distances from the clones.

TABLE 19

| | | Distance from | Distance from |
|---|---|---|---|
| Clone | Chromosome | Marker, cR | Marker, cR |
| 20468752.0.18 | 11 | WI-6150, 2.8 cR | WI-5256, 3.8 cR |
| 11692010.0.51 | 20 | D20S172, 3.9 cR | NIB1603,7.5 cR |

### EXAMPLE 2

### Molecular Cloning of a Clone 20468752.0.18-U, PRO2 Nucleic Acid

The cDNAs coding for both the full-length 720 residue protein predicted for Clone 20468752.0.18-U (a PRO 2 nucleic acid) and the mature polypeptide with the 21 residue signal peptide removed were targeted for cloning.

A. Mature Protein

The following oligonucleotide primers were used to clone the cDNA coding for the mature form:

| | | |
|---|---|---|
| 20468752 Eco Forward: | GAA TTC TTG CCA AGA GAG TAC ACA GTC ATT AAT G | (SEQ ID NO:35) |
| 20468752 Hind Forward: | AAG CTTTTGCCAAGAGAGTACACAGTCATTAATG | (SEQ ID NO:36) |
| 20468752 New Reverse: | CTC GAG TTT CAT ATT TCT TTC AAT CCA GTC | (SEQ ID NO:37) |

For downstream cloning purposes, the forward primers include either an in frame EcoRI or HindIII restriction site, whereas the reverse primer contains an in frame XhoI restriction site.

A PCR amplification reaction was performed using a total of 5 ng of human placenta cDNA as template. The reaction mixtures contained the following reagents: 1 μM of each of the 20468752 Eco Forward or 20468752 Hind Forward primers in combination with the 20468752 New Reverse primer; 5 μmoles of dNTP mixture (Clontech Laboratories; Palo Alto, Calif.) and 1 μl of 50× Advantage-HF 2 polymerase (Clontech Laboratories; Palo Alto, Calif.) in a 50 μl total reaction volume. The following PCR amplification reaction conditions were used:

(a) 96° C. 3 minutes

(b) 96° C. 30 seconds denaturation

(c) 60° C. 30 seconds, primer annealing

(d) 72° C. 4 minute extension

Repeat steps (b)-(d) a total of 35-times

e) 72° C. 5 minutes final extension

An amplified product having the expected size of approximately 2 kbp was detected by agarose gel electrophoresis. The fragment was then purified from the agarose gel and ligated to the pCR2.1 vector (Invitrogen; Carlsbad, Calif.) following the manufacturer's recommendation. The cloned insert was sequenced, using vector-specific M13 Forward and M13 Reverse primers in combination with the following gene-specific primers:

| | | |
|---|---|---|
| 20468752 Seq1: | TGT GGC CAG GTT CTG CGA | (SEQ ID NO:38) |
| 20468752 Seq2: | CTT GAC AAG GCT GGA TCT | (SEQ ID NO:39) |
| 20468752 Seq3: | CCT ACC AAG AAG CCA GCC | (SEQ ID NO:40) |
| 20468752 Seq4: | TCG CAG AAC CTG GCC ACA | (SEQ ID NO:41) |
| 20468752 Seq5: | AGA TCC AGC CTT GTC AAG | (SEQ ID NO:42) |
| 20468752 Seq6: | GGC TGG CTT CTT GGT AGG | (SEQ ID NO:43) |
| 20468752 S7: | CAG GCA GCC ATC TAC AGG AGG | (SEQ ID NO:44) |
| 20468752 S8: | CCT CCT GTA GAT GGC TGC CTG | (SEQ ID NO:45) |
| 20468752 S9: | CAG GAG TCC CAC ATC ACT | (SEQ ID NO:46) |
| 20468752 S10: | AGT GAT GTG GGA CTC CTG | (SEQ ID NO:47) |

The insert was verified as an open reading frame (ORF) coding for the predicted 20468752.0.18-U protein (PROX 2) between residues 22 and 720. The translated amino acid sequence is 100% identical to that predicted for the mature form of clone 20468752.0.18-U. The construct was designated pCR2.1-20468752-S414A.

B. Full-Length Clone 20468752.0.18-U

In order to clone the full-length cDNA, PCR primers were designed to amplify the 5′ portion of the cDNA from the ATG start site to a unique BamHI site. The following primers were used:

| | | |
|---|---|---|
| 20468752 Nat Forw: | GCTAGCCACCATGGAGCTGGGTTGCTGGACGCAGTTGG | (SEQ ID NO:48) |
| 20468752 Nat Rev: | AGGACGTGGAGTGAGGATCCTATGCTCTGGATAGG | (SEQ ID NO:49) |

The forward primer contains an NheI restriction site and a consensus Kozak sequence (CCACC). The reverse primer spans the region that contains a BamHI restriction site in position 759 of the cDNA sequence.

A PCR amplification reaction was performed using a total of 5 ng of human placenta cDNA as template. The reaction mixtures contained the following reagents: 1 μM of each of the 20468752 Nat Forw primer in combination with the 20468752 Nat Rev primer; 5 μmoles of dNTP mixture (Clontech Laboratories; Palo Alto, Calif.); and 1 μl of 50× Advantage-HF 2 polymerase (Clontech Laboratories; Palo Alto, Calif.) in a 50 μl total reaction volume. The reaction conditions were the same as set forth above, except that the extension time in step (d) was 2 minutes.

An amplified product having the expected size was detected by agarose gel electrophoresis. The PCR product was then isolated from the agarose gel and cloned into the pCR2.1 vector. The sequence of the construct was verified as the 5′ segment of Clone 20468752 from the ATG start site spanning to the BamHI-759 site. The resulting construct was designated called pCR2.1-20468752-Nat-S530-17C.

The expression construct containing the mature 20468752.0.18-U segment (designated pCEP4/Sec-20468752; see, Example 4, infra) was digested with NheI and BamHI and the linearized vector was gel purified. pCR2.1-20468752-Nat-S530-17C was also digested with NheI and BamHI, and the resulting fragment (which contained the ATG start site up to the BamHI-759 site) was isolated. This fragment was subsequently ligated to the linearized expression vector. The sequence of the cloned polynucleotide was found to encode a polypeptide whose sequence is identical to that predicted for the protein encoded by Clone 20468752.0.18-U, from residue 1 to residue 678.

### EXAMPLE 3

### Preparation of Mammalian Expression Vector pCEP4/Sec

Two oligonucleotide primers were designed to amplify a fragment from the pcDNA3.1-V5His (Invitrogen, Carlsbad, Calif.) expression vector that includes V5 and His6. These primers include:

| | | |
|---|---|---|
| pSec-V5-His Forward: | CTCGTCCTCGAGGGTAAGCCTATCCCTAAC | (SEQ ID NO:50) |
| pSec-V5-His Reverse: | CTCGTCGGGCCCCTGATCAGCGGGTTTAAAC | (SEQ ID NO:51) |

Following PCR amplification, the product was digested with XhoI and ApaI and ligated into the XhoI/ApaI-digested pSecTag2 B vector harboring an i kappa leader sequence (Invitrogen; Carlsbad, Calif.). The correct structure of the resulting vector (designated pSecV5His), including an in-frame i-kappa leader and V5-His6, was verified by DNA sequence analysis. The vector pSecV5His was then digested with PmeI and NheI to provide a fragment retaining the above elements in the correct frame. The PmeI/NheI-digested fragment was ligated into the BamHI/Klenow- and NheI-treated vector pCEP4 (Invitrogen; Carlsbad, Calif.). The resulting vector was designated pCEP4/Sec, and included an in-frame i kappa leader, a site for insertion of a clone of interest, and V5 and His6 sites under control of the PCMV and/or the PT7 promoter. pCEP4/Sec is an expression vector that allows heterologous protein expression and secretion by fusing any protein to the i Kappa chain signal peptide. Detection and purification of the expressed protein was aided by the presence of the V5 epitope tag and 6× His tag at the carboxyl-terminus (Invitrogen; Carlsbad, Calif.).

### EXAMPLE 4

### Expression of 20468752.0.18-U in Human Embryonic Kidney 293 Cells

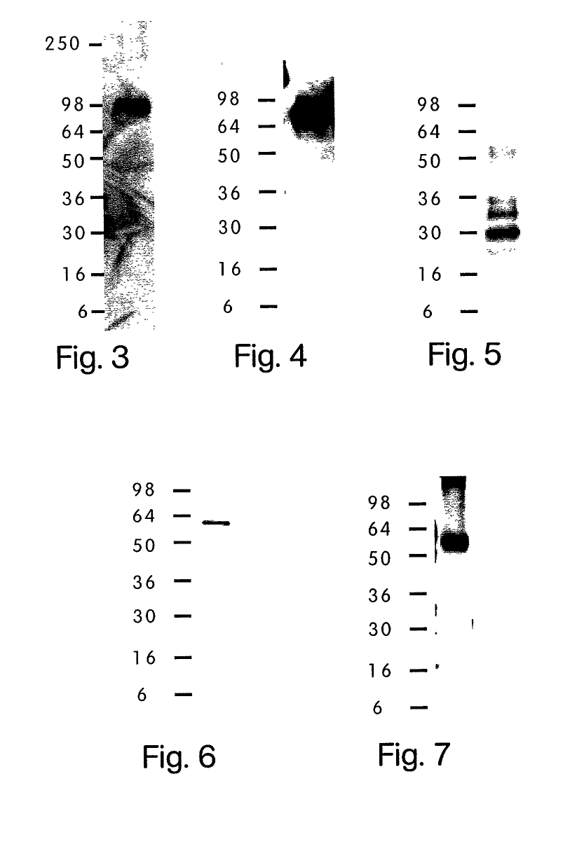

The EcoRI-XhoI fragment containing the mature 20468752.0.18-U sequence was isolated from pCR2.1-20468752-S414A (Example 2, supra) and subcloned into the vector pET28a (Novagen; Madison, Wis.). The resulting vector (designated pET28a-20468752) was partially-digested with BamHI, and then completely-digested with XhoI. The resulting 2.0 kb fragment was isolated and ligated into BamHI-XhoI digested pCEP4/Sec (see, Example AB3, supra) to generate an expression vector designated pCEP4/Sec-20468752. The pCEP4/Sec-20468752 vector was subsequently transfected into Human Embryonic Kidney 293 cells using the LipofectaminePlus® reagent following the manufacturer's instructions (Gibco/BRL; Rockville, Md.). The cell pellet and supernatant were harvested approximately 72 hours after transfection and examined for h20468752 expression by Western blotting (under reducing conditions) with an anti-V5 antibody. FIG. 3 shows that the mature 20468752.0.18-U is expressed as a protein with an apparent molecular weight (Mr) of approximately 9800Daltons which is secreted by the 293 cells.

### EXAMPLE 5

### Molecular Cloning of 11692010.0.51

The predicted open reading frame (ORF) of Clone 11692010.0.51 encodes a 649 amino acid Type Ia transmembrane protein. The SIGNALP computer program predicted a signal sequence, with a peptidase cleavage site most likely located between residues 28 and 29. The PSORT computer program predicted the transmembrane region to be located between residues 532 and 548. Therefore, a cDNA encoding the mature form of the extracellular segment (i.e., between residues 29 and 531) was selected for subsequent cloning. The following oligonucleotide primers were designed to PCR amplify this cDNA:

| | | |
|---|---|---|
| 11692010 Forward: | GGATCC AAA TCC TGT CCA TCT GTG TGT CGC TG | (SEQ ID NO:52) |
| 11692010 Reverse: | CTCGAG AGC CAA AGG TAA ATT GGG GTT TTT GTA AG | (SEQ ID NO: 53) |

For downstream cloning purposes, the forward primer included an in-frame BamHI restriction site, whereas the reverse primer contained an in-frame XhoI restriction site. In the sequences for 11692010 Forward and 11692010 Reverse, above, the restriction site sequences are underlined.

A PCR amplification reaction was performed using a total of 5 ng of human fetal brain cDNA as template. The reaction mixtures contained the following reagents: 1 μM each, of the 11692010 Forward and 11692010 Reverse primers; 5 μmoles of dNTP mixture (Clontech Laboratories; Palo Alto, Calif.) and 1 μl of 50× Advantage-HF 2 polymerase (Clontech Laboratories; Palo Alto, Calif.) in a 50 μl total reaction volume. The reaction conditions as previously described in Example 2, Section B) were utilized.

An amplified product, having the expected size of approximately 1500 bp, was detected by agarose gel electrophoresis. The fragment was purified from the gel, and ligated into the pCR2.1 vector (Invitrogen; Carlsbad, Calif.) following the manufacturer's recommendation. The cloned insert was then sequenced (using vector-specific M13 Forward and M13 Reverse primers) in combination with the following gene-specific primers:

| | |
|---|---|
| 11692010 Seq1: | |
| CGA GAC AGC AAC TAT CTC | (SEQ ID NO:54) |
| 11692010 Seq2: | |
| CGA CTG GAT ATG TCC AAT | (SEQ ID NO:55) |
| 11692010 Seq3: | |
| ACA ATT ACT GTG AAG TCT | (SEQ ID NO:56) |
| 11692010 Seq4: | |
| GAG ATA GTT GCT GTC TCG | (SEQ ID NO:57) |
| 11692010 Seq5: | |
| ATT GGA CAT ATC CAG TCG | (SEQ ID NO:58) |
| 11692010 Seq6: | |
| AGA CTT CAC AGT AAT TGT | (SEQ ID NO:59) |

The insert was verified as an open reading frame (ORF) encoding the predicted 11692010.0.51 protein between residues 29 and 351. The construct was designated 11692010.0.51-pCR2.1-S214-3C. The translated protein sequence encoded by this construct was found to be 100% identical to the corresponding portion of Clone 11692010.0.51.

### EXAMPLE 6

### Expression of 11692010.0.51 in Human Embryonic Kidney 293 Cells

The BamHI/XhoI fragment containing the cloned fragment of the 11692010.0.51 sequence was isolated from the 11692010-in pCR2.1 vector-S214-3C (see, Example 5, supra) and subcloned into BamHI/XhoI-digested pCEP4/Sec (see, Example 3, supra) to generate an expression vector designated CEP41Sec-11692010. The pCEP41Sec-11692010 construct was then transfected into Human Embryonic Kidney 293 cells using the LipofectaminePlus® reagent following the manufacturer's instructions (Gibco/BRL; Rockville, Md.). The cell pellet and supernatant were harvested approximately 72 hours after transfection and examined for 11692010 expression by Western blotting (under reducing conditions) with an anti-V5 antibody. FIG. 4 shows that 11692010 is expressed as a protein with a Mr of approximately 80000 Daltons which is secreted by the 293 cells.

### EXAMPLE 7

### Molecular Cloning of Clone 27835981.0.1, PRO4 Nucleic Acid

Oligonucleotide primers were designed to PCR amplify a DNA segment, representing an ORF, encoding the mature form of the 27835981.0.1 protein (i.e., from residues 25 to 160). The forward primer included an in-frame BamHI restriction site, whereas the reverse primer contained an in-frame XhoI restriction site. These primers had the following sequences:

| | | |
|---|---|---|
| 27835981 Forward: | GGATCC GAG GCT GAA GGC AAT GCA AGC TGC ACA G | (SEQ ID NO:60) |
| 27835981 Reverse: | TCGAG CAG TGG AAT GTA GGT GCT GTG AAT GCA G | (SEQ ID NO:61) |

PCR amplification reactions were performed using 5 ng of human pancreas cDNA template; 1 μM of each of the 27835981 Forward primer (SEQ ID NO:85) and 27835981 Reverse primer (SEQ ID NO:87); 5 μmoles of dNTP mixture (Clontech Laboratories; Palo Alto, Calif.); and 1 μl of 50× Advantage-HF 2 polymerase (Clontech Laboratories; Palo Alto, Calif.) in a 50 μl total reaction volume. The following PCR amplification reaction conditions were used:

| | | |
|---|---|---|
| (a) | 96° C. | 3 minutes |
| (b) | 96° C. | 30 seconds denaturation |
| (c) | 70° C. | 30 seconds, primer annealing. This temperature was |
| | | gradually decreased by 1° C./cycle |
| (d) | 72° C. | 1 minute extension. |
| Repeat steps (b)-(d) a total of 10-times | | |
| (e) | 96° C. | 30 seconds denaturation |
| (f) | 60° C. | 30 seconds annealing |
| (g) | 72° C. | 1 minute extension |
| Repeat steps (e)-(g) a total of 25-times | | |
| (h) | 72° C. | 5 minutes, final extension |

An amplified product, having a size of approximately 400 bp, was detected by agarose gel electrophoresis. The product was then isolated by use of the QIAEX II ® Gel Extraction System (QUIAGEN, Inc; Valencia, Calif.) in a final volume of 20 μl.

The isolated product was subsequently ligated into the pCR2.1 vector and sequenced. The sequence verified that the insert was as an ORF encoding a sequence which was 100% identical to the mature 27835981.0.1 protein. The construct was designated pCR2.1-27835981-S216.

### EXAMPLE 8

### Expression of 27835981.0.1 in Human Embryonic Kidney 293 Cells

The BamHI/XhoI fragment containing the 27835981.0.1 sequence was isolated from the pCR2.1-27835981-S216 construct (see, Example 7, supra) and subcloned into BamHI/XhoI-digested pCEP4/Sec (see, Example 3, supra) to generate a new construct designated pCEP4/Sec-27835981. The pCEP4/Sec-27835981 construct was then transfected into Human Embryonic Kidney 293 cells using the LipofectaminePlus® reagent following the manufacturer's instructions (Gibco/BRL; Rockville, Md.). The cell pellet and supernatant were harvested approximately 72 hours after transfection and examined for 27835981.0.1 expression by Western blotting (under reducing conditions) with an anti-V5 antibody. FIG. 5 shows that 27835981.0.1 is expressed as a protein with an approximate Mr of 30000 Daltons and is secreted by the 293 cells.

### EXAMPLE 9

### Molecular Cloning of Clone 21399247.0.1, a PRO5 nucleic acid

The predicted open reading frame (ORF) of Clone 21399247.0.1 encodes a 580 amino acid residue protein. The SIGNALP computer program predicted a secretory signal sequence, with a cleavage site most likely located between residues 16 and 17. Oligonucleotide primers were designed to PCR amplify a DNA segment, representing the ORF, encoding the mature 21399247.0.1 protein (i.e., from residues 17 to 580). The forward primer included an in-frame BamHI restriction site, whereas the reverse primer contained an in-frame XhoI restriction site. The primers had the following sequences:

| | | |
|---|---|---|
| 21399247 Forward: | GGATCC GCG GTC CTG TGG AAG CAT GTG CGG CTG | (SEQ ID NO:62) |
| 21399247 Reverse: | CTCGAG CGT GTT GCA CAC CAG CAC ATC TGC | (SEQ ID NO:63) |

PCR amplification reactions were performed using 5 ng of human thyroid cDNA template; 1 μM each of the 21399247 Forward (SEQ ID NO:89) and the 21399247 Reverse primer (SEQ ID NO:91); 5 μmoles of dNTP mixture (Clontech Laboratories; Palo Alto, Calif.); and 1 μl of 50× Advantage-HF 2 polymerase (Clontech Laboratories; Palo Alto, Calif.) in a 50 μl total reaction volume. The amplification reaction conditions were the same as those used in Example 7, with the exception of the extensions in steps (d) and (g) were performed for 3 minutes.

A 1.7 kbp amplification product was detected by agarose gel electrophoresis. The product was isolated using the QIAEX II Gel Extraction System® (QUIAGEN, Inc; Valencia, Calif.) in a final total volume of 20 μl.

The isolated product was ligated into pCR2.1 vector and sequenced using vector specific and the following gene specific primers:

| | | |
|---|---|---|
| 21399247 Seq1: | GAC GTG GCC CTC ATC GCC AAC | (SEQ ID NO:64) |
| 21399247 Seq2: | CTA GGC GAG GAG TAC ATT CTG | (SEQ ID NO:65) |
| 21399247 Seq3: | CTG GAC CGG GCT GAG CAA | (SEQ ID NO:66) |
| 21399247 Seq4: | GTT GGC GAT GAG GGC CAC GTC | (SEQ ID NO:67) |
| 21399247 Seq5: | CAG AAT GTA CTC CTC GCC TAG | (SEQ ID NO:68) |
| 21399247 Seq6: | TTG CTC AGC CCG GTC CAG | (SEQ ID NO:69) |

The sequence analysis verified that the insert was an ORF encoding a polypeptide that is 100% identical to the corresponding mature 21399247.0.1 protein. The construct was designated pCR2.1-21399247-S203#15.

### EXAMPLE 10

### Expression of 21399247.0.1 in Human Embryonic Kidney 293 Cells

The BamHI/XhoI fragment containing the mature 21399247.0.1 sequence was isolated from the pCR2.1-21399247-S203#15 construct (see, Example 9, supra) and subcloned into BamHI/XhoI-digested pCEP4/Sec (see, Example 3, infra) to generate a new construct designated pCEP4/Sec-21399247. The pCEP4/Sec-21399247 construct was then transfected into Human Embryonic Kidney 293 cells using the LipofectaminePlus reagent® following the manufacturer's instructions (Gibco/BRL; Rockville, Md.). The cell pellet and supernatant were harvested approximately 72 hours after transfection and examined for expression of 21399247.0.1 by Western blotting (under reducing conditions) with an anti-V5 antibody. FIG. 6 shows that 21399247.0.1 is expressed as a protein with a Mr of approximately 62000 Daltons and is secreted by the 293 cells.

### EXAMPLE 11

### Molecular Cloning of Clonel17941787.0.1, aPRO14 Nucleic Acid

The predicted open reading frame (ORF) of Clone 17941787.0.1 was shown to encode a protein of 840 amino acid residues. The SIGNALP computer program predicted a secretory signal sequence, with a cleavage site most-likely located between amino acid residues 27 and 28. The PSORT computer program predicted a transmembrane domain, located between amino acid residues 477 and 493. Oligonucleotide primers were then designed to PCR amplify a DNA segment encoding the mature 17941787.0.1 protein (i.e., from amino acid residues 28 to 476). The forward primer included an in-frame KpnI restriction site, whereas the reverse primer contained an in-frame XhoI restriction site. The primers had the following sequences:

| | | |
|---|---|---|
| 17941787 Forward: | GGT ACC TGT GGA GAG ACT CCA GAG CAA ATA CGA | (SEQ ID NO:70) |
| 17941787 Reverse: | CTC GAG AGT GAT GAC TCT TGT AGG CAC GAT TAC | (SEQ ID NO:71) |

PCR amplification reactions were performed using 5 ng of human mammary gland cDNA template; 1 μM each of the 17941787 Forward (SEQ ID NO:105) and the 17941787 Reverse primer (SEQ ID NO:107); 5 μmoles of a dNTP mixture (Clontech Laboratories; Palo Alto, Calif.); and 1 μl of 50× Advantage-HF 2 polymerase (Clontech Laboratories; Palo Alto, Calif.) in a 50 μl total reaction volume. The PCR amplification reaction conditions were identical to those utilized in Example 9.

A PCR amplification product having a size of approximately 1.3 kbp was detected by agarose gel electrophoresis. The product was isolated by use of the QIAEX II Gel Extraction System® (QUIAGEN, Inc; Valencia, Calif.) in a final volume of 20 μl.

The isolated PCR amplification product was then ligated into the pCR2.1 vector and sequenced concomitant use of both vector-specific and gene specific primers. The sequences of the gene-specific primers were as follows:

| | |
|---|---|
| 17941787 Seq1: | |
| GCT TGT GAT CAG TTT CGT | (SEQ ID NO:72) |
| 17941787 Seq2: | |
| TGC ACC TGG TTA ATA GAC | (SEQ ID NO:73) |
| 17941787 Seq3: | |
| ACT GAG CAG CAG CGT TGT | (SEQ ID NO:74) |
| 17941787 Seq4: | |
| ACG AAA CTG ATC ACA AGC | (SEQ ID NO:75) |
| 17941787 Seq5: | |
| TAT TAA CCA GGT GCA ATT | (SEQ ID NO:76) |
| 17941787 Seq6: | |
| ACA ACG CTG CTG CTC AGT | (SEQ ID NO:77) |

The sequence obtained by DNA sequence analysis verified the insert as being an ORF that was 100% identical to the mature 17941787.0.1. The construct was designated pCR2.1-17941787-S323-6C.

### EXAMPLE 12

### Expression of 17941787.0.1 in Human Embryonic Kidney 293 Cells

The KpnI/XhoI fragment containing the 17941787.0.1 sequence was isolated from the pCR2.1-17941787-S323-6C construct (see, Example 11, supra) and then subcloned into KpnI/XhoI-digested pCEP4/Sec (see, Example 3, supra) to generate the new construct pCEP4/Sec-17941787. The pCEP4/Sec-17941787 construct was subsequently transfected into Human Embryonic Kidney 293 cells using the LipofectaminePlus reagent® following the manufacturer's instructions (Gibco/BRL; Rockville, Md.). The cell pellet and supernatant were harvested approximately 72 hours after transfection and examined for 17941787.0.1 expression by Western blotting (under reducing conditions) with an anti-V5 antibody. FIG. 7 shows that 17941787.0.1 is expressed intracellularly as a protein having a Mr of approximately 55 kDa by the 293 cells.

### EXAMPLE 13

### Molecular Cloning of Clone 16467945.0.85, a PRO16 Nucleic Acid, and Clone 16467945.0.88, a PRO17 Nucleic Acid

A. Cloning of Mature Soluble 16467945.0.85

The predicted open reading frame (ORF) encodes a protein comprising 123 amino acid residues. The SIGNALP computer program predicted a secretory signal sequence, with a cleavage site most-likely located between amino acid residues 19 and 20. Accordingly, oligonucleotide primers were designed to PCR amplify a DNA segment encoding the mature 16467945.0.85 (i.e., from amino acid residues 20 to 123). The forward primer included an in-frame BamHI restriction site and the reverse primer contains an in frame XhoI restriction site. The sequences of the primers are the following:

| | | |
|---|---|---|
| 16467945.8588 Forward: | GGATCC GAG TAC GAC GGG AGG TGG CCC AGG | (SEQ ID NO:78) |
| 16467945.85 Reverse: | CTCGAG CAG GGT AGA GCC ACG GCG CCC GGC TGG AAC | (SEQ ID NO:79) |

PCR amplification reactions were performed using 5 ng of human fetal lung cDNA template; 1 μM each of the 16467945.8588 Forward primer and the 16467945.85 Reverse primer; 5 μmoles of a dNTP mixture (Clontech Laboratories; Palo Alto, Calif.); and 1 μl of 50× Advantage-HF 2 polymerase (Clontech Laboratories; Palo Alto, Calif.) in 50 μl total reaction volume. The PCR amplification reaction were identical to those utilized in Example 9.

An amplification product having a size of approximately 300 bp was detected by agarose gel electrophoresis. The product was isolated by use of the QIAEX II Gel Extraction System® (QUIAGEN, Inc; Valencia, Calif.) in a final volume of 20 μl.

The isolated PCR amplification product was then ligated into the pCR2.1 vector and sequenced using vector-specific primers. The nucleotide sequence which was obtained, as well as the amino acid sequence of the translated polypeptide are shown in Table 20.

TABLE 20
(1) Nucleic Acid Sequence of 16467945.0.85-S259.A:

| | |
|---|---|
| GAGTACGACGGGAGGTGGCCCAGGCAAATAGTGTCATCGATTGGCCTATGTCGTTATGGTGGGAGGATTGACTGC | (SEQ ID NO:80) |
| TGCTGGGGCTGGGCTCGCCAGTCTTGGGGACAGTGTCAGCCTGTGTGCCAACCACGATGCAACATGGTGAATGTA | |
| TCGGGCCAAACAAGTGCAAGTGTCATCCTGGTTATGCTGGAAAAACCTGTAATCAAGCCGTAGGTTTTGAAAGAT | |
| GTATGGTTCCAGCCGCGCGCCGTGGCTCTACCCTG | |

(2) Amino Acid Sequence of 16467945.0.85-S259.A:

EYDGRWPRQIVSSIGLCRYGGRIDCCWGWARQSWGQCQPVCQPRCKHGECIGPNKCKCHPGYAGKTCNQAVGFERC MVPAGRRGSTL (SEQ ID NO:81)

The nucleic acid sequencing verified the insert as an ORF encoding the mature 16467945.85. The construct was designated pCR2.1-16467945.85 -S259A.

B. Cloning Mature 16467945.0.88

The identical PCR conditions which used to amplify 16467945.0.88 were used in the amplification of 16467945.0.88. The resulting construct was designated 16467945.0.88-S261.D. The nucleotide sequence (SEQ ID NO:81) and the amino acid sequence (SEQ ID NO:82) are presented below in Table 21.

TABLE 21
(1) Nucleic Acid Sequence of 16467945.O.88-S261.D

| | | |
|---|---|---|
| 1 | GAGTTCGACGGGAGGTGGCCCAGGCAAATAGTGTCATCGATTGGCCTATGTCGTTATGGTGGGAGGATTGACTGCTGCTG | (SEQ ID NO:81) |
| 81 | GGGCTGGGCTCGCCAGTCTTGGGGACAGTGTCAGCCTGTGTGCCAACCACGATGCAAACATGGTGAATGTATCGGGCCAA | |
| 161 | ACAAGTGCAAGTGTCATCCTGGTTATGCTGGAAAAACCTGTATTCAAGTTTTAAATGAGTGTGGCCTGAAGCCCCGGCCC | |
| 241 | TGTAAGCACAGGTGCATGAACACTTACGGCAGCTACAAGTGCTACTGTCTCAACGGATATATGCTCATGCCGGATGGTTC | |
| 321 | CTGCTCAAGTGCCCTGACCTGCTCCATGGCAAACTGTCAGTATGGCTGTGATGTTGTTAAAGGACAAATACGGTGCCAGT | |
| 401 | GCCCATCCCCTCGCCTGCAGCTGGCTCCTGATGGGAGGACCTGTGTAGATGTTGATGAATGTGCTACAGGAAGAGCCTCC | |
| 481 | TGCCCTAGATTTAGGCAATGTGTCAACACTTTTGGGAGCTACATCTGCAAGTGTCATAAAGGCTTCGATCTCATGTATAT | |
| 561 | TGGAGGCAAATATCAATGTCATGACATAGACGAATGCTCACTTGGTCAGTATCAGTGCAGCAGCTTTGCTCGATGTTATA | |
| 641 | ACGTACGTGGGTCCTACAAGTGCAAATGTAAAGAAGGATACCAGGGTGATGGACTGACTTGTGTGTATATCCCAAAAGTT | |
| 721 | ATGATTGAACCTTCAGGTCCAATTCATGTACCAAAGGGAAATGGTACCATTTTAAAGGGTGACACAGGAAATAATAATTG | |
| 801 | OATTCCTGATGTTGGAAGTACTTGGTGGCCTCCGAAGACACCATATATTCCTCCTATCATTACCAACAOGCCTACTTCTA | |
| 881 | AGCCAACAACAAGACCTACACCAAAGCCAACACCAATTCCTACTCCACCACCACCACCACCCCTGCCAACAGAGCTCAGA | |
| 961 | ACACCTCTACCACCTACAACCCCAGAAAGGCCAACCACCGGACTGACAACTATAGCACCAGCTGCCAGTACACCTCCAGG | |
| 1041 | AGGGATTACAGTTGACAACAGGGTACAGACAGACCCTCAGAAACCCAGAGGAGATGTGTTCATTCCACGGCAACCTTCAA | |
| 1121 | ATGACTTGTTTGAAATATTTGAAATAGAAAGAGGAGTCAGTGCAGACGATGAAGCAAAGGATGATCCAGGTGTTCTGGTA | |
| 1201 | CACAGTTGTAATTTTGACCATGGACTTTGTGGATGGATCAGGGAGAAAGACAATGACTTGCACTGGGAACCAATCAGGGA | |
| 1281 | CCCAGCAGGTGGACAATATCTGACAGTGTCGGCAGCCAAAGCCCCAGGGGGAAAAGCTGCACGCTTGGTGCTACCTCTCG | |
| 1361 | GCCGCCTTATGCATTCAGGGGACCTGTGCCTGTCATTCAGGCACAAGGTGACGOGGCTGCACTCTGGCACACTCCAGGTG | |
| 1441 | TTTGTGAOAAAACACOGTGCCCACOGAGCAGCCCTGTGGGGAAGAAATGGTGGCCATGGCTGGAGOCAAACACAGATCAC | |
| 1521 | CTTGCGAGGGGCTGACATCAAGAGCGTCGTCTTCAAAGGTGAAAAAAGGCGTGGTCACACTGGGOAGATTGGATTAGATG | |
| 1601 | ATGTGAGCTTGAAAAAAGGCCACTGCTCTGAAGAACGC | |

(2) Amino Acid Sequence of 16467945.O.88-S261.D

| | | |
|---|---|---|
| 1 | EYDGRWPRQIVSSIGLCRYGGRIDCCWGWARQSWGQCQPVCQPRCKEGECIGPNKCKCHPGYAGKTCIQVLNECGLKPRP | (SEQ ID NO:82) |
| 81 | CKHRCMNTYGSYKCYCLNGYMLMPDGSCSSALTCSMANCQYGCDVVKGQIRCQCPSPGLQLAPDGRTCVDVDECATGRAS | |
| 161 | CPRFRQCVNTFGSYICKCHKGFDLMYIGGKYQCHDIDECSLGQYQCSSFARCYNVRGSYKCKCKEGYQGDGLTCVYIPKV | |
| 241 | MIEPSGPIUVPKGNGTILKGDTGNNNWIPDVOSTWWPPKTPYIPPIITNRPTSKPTTRPTPKPTPIPTPPPPPPLPTELR | |
| 321 | TPLPPTTPERPTTGLTTIAPAASTPPGGITVDNRVQTDPQKPRGDVFIPRQPSNDLFEIFEIERGVSADDEAXDDPGVLV | |
| 401 | HSCNFDHGLCGWIREKDNDLEWEPIRDPAGOQYLTVSAAKAPGGKAARLVLPLGRLMHSGDLCLSFRHKVTGLHSGTLQV | |
| 481 | FVRKHGAHGAALWGRNGGHGWRQTQITLRGADIKSVVFKGEKRRGHTGEIGLDDVSLKKGECSEER | |

While the nucleic acid and amino acid sequences of 16467945.0.85-S259.A and the nucleic acid and amino acid sequences of 16467945.0.88-S261.D overlap with one another, both sets of sequences confirm that they represent a splice variant with respect to the nucleic acid and amino acid sequences presented above for Clone 16467945.0.85 and Clone 16467945.0.88 (SEQ ID NO:33 and SEQ ID NO:34, respectively). Specifically, the results of the molecular cloning in the present Example (i.e., construct 16467945.0.85-S259.A and construct 16467945.0.88-S261.D) include a deletion when compared to the sequences of Clone 16467945.0.85 and Clone 16467945.0.88. This relationship is pictorially-shown below. It should be noted that only the region of sequence which includes the deletion is shown, below.

EYDGRWPRQIVSSIGLCRYGGRIDCCWGWARQSWGQCQPFYVLRQRIARIRCQLKAVCQPR
::::::::::::::::::::::::::::::::::::::: :::::
EYDGRWPRQIVSSIGLCRYGGRIDCCWGWARQSWCQCQP-----------------VCQPR

### EXAMPLE 14

### Expression of 16467945.0.88 in Human Embryonic Kidney 293 Cells

The KpnI/XhoI fragment containing the 16467945.0.88 sequence (see, Example 13, supra) was isolated from 16467945.0.88-in pCR2.1 vector (i.e., S323-6c) and subcloned into BamHI/XhoI-digested pCEP4/Sec (see, Example 3, supra) to generate the new construct pCEP4/Sec-16467945.0.88. The pCEP4/Sec-16467945.0.88 construct was then transfected into Human Embryonic Kidney 293 cells using the LipofectaminePlus reagent® following the manufacturer's instructions (Gibco/BRL; Rockville, Md). The cell pellet and supernatant were harvested approximately 72 hours after transfection and examined for 16467945.0.88 expression by Western blotting (under reducing conditions) with an anti-V5 antibody. FIG. AG2 shows that 16467945.0.88 is expressed as two proteins with molecular weights of approximately 95000 Daltons and 23000 Daltons, as secreted by the 293 cells. The 23000 Dalton protein is believed to be a degradation product of the 95000 Dalton protein.

### EXAMPLE 15

### Quantitative Analysis of the Tissue Distribution of Expression of PROX Nucleic Acids

The quantitative expression of various clones of the invention was assessed in 41 normal and 55 tumor samples (identified in the Tables that follow) by real-time quantitative PCR analysis (TAQMAN®) performed on a Perkin-Elmer Biosystems ABI PRISM® 7700 Sequence Detection System.

In the following Tables, these abbreviations are used:

ca.=carcinoma

*=established from metastasis

met=metastasis

s cell var=small cell variant

non-s=non-sm=non-small

squam=squamous

pl. eff=pl effusion=pleural effusion

glio=glioma

astro=astrocytoma

neuro=neuroblastoma

In this analysis, 96 RNA samples were initially normalized to β-actin and GAPDH. RNA (˜50 ng total or ˜1 ng poly(A)+) was converted to cDNA using the TAQMAN® Reverse Transcription Reagents Kit (PE Biosystems; Foster City, Calif.; Catalog No. N808-0234) and random hexamers, according to the manufacturer's protocols. Reactions were performed in 20 μl total reaction volumes and incubated for 30 minutes at 48° C. cDNA (5 μl) was then transferred to a separate plate for the TAQMAN® reaction using β-actin and GAPDH TAQMAN® Assay Reagents (PE Biosystems; Catalog No. 4310881E and No. 4310884E, respectively) and TAQMAN® Universal PCR Master Mix (PE Biosystems; Catalog No. 4304447), according to the manufacturer's protocol. Reactions were performed in a 25 μl reaction volume using the following parameters: 2 minutes at 50° C.; 10 minutes at 95° C.; and 15 seconds at 95° C./1 minute at 60° C. (for a total of 40 cycles). Results were recorded as CT values (cycle at which a given sample crosses a threshold level of fluorescence) using a logarithmic scale. The difference in RNA concentration between a given sample and the sample with the lowest CT value was represented as 2 to the power of delta CT (i e., 2^{δCT}). The percent relative expression was then obtained by taking the reciprocal of this RNA difference and multiplying by 100. The average CT values obtained for β-actin and GAPDH were used to normalize the RNA samples. The RNA sample which generated the highest CT value required no further diluting, while all other samples were diluted relative to this sample according to their specific β-actin /GAPDH average CT values.

Normalized RNA (5 μl ) was converted to cDNA and analyzed via TAQMAN® using One Step RT-PCR Master Mix Reagents (PE Biosystems; Catalog No 4309169) and gene-specific primers, according to the manufacturer's protocols. Probes and primers were designed for each assay according to Perkin Elmer Biosystem's Primer Express Software package (Version I for Apple Computer's Macintosh Power PC) using the sequence of Clone 10326230.0.38 as input. Default settings were used for reaction conditions and various parameters were set before selecting the primers to be utilized. These parameters included: primer concentration=250 nM; primer melting temperature (Tₘ) range=58°-60° C.; primer optimal Tₘ=59° C.; maximum primer difference=2° C. (when the probe does not have 5′-terminal G, the probe Tₘ must be 10° C. greater than the primer Tₘ); and amplicon size=75 bp to 100 bp. The probes and primers which were selected (see, infra) were synthesized by Synthegen (Houston, Tex.). Probes were double-purified by HPLC to remove uncoupled dye, and then evaluated by mass spectroscopy to verify coupling of reporter and quencher dyes to the 5′- and 3′-termini of the probe, respectively. The final probe concentrations were: forward and reverse primers=900 nM each; and probe=200 nM.

The following PCR amplification reaction conditions were utilized. Normalized RNA from each tissue and cell line type was then spotted in the individual wells of a 96-well PCR plate (Perkin Elmer Biosystems). The PCR amplification reaction mixtures included the following reagents: 2 probes (an PROX-specific and another gene-specific probe multiplexed with the PROX-specific); 1× TaqMan™ PCR Master Mix for the PE Biosystems 7700; 5 mM MgCl₂; dNTP mixture (dA, G, C, U at 1:1:1:2 ratios); 0.25 U/ml AmpliTaq Gold™ (PE Biosystems); 0.4 U/μl RNase inhibitor; and 0.25 U/μt reverse transcriptase. Reverse transcription was then performed at 48° C. for 30 minutes followed by PCR amplification cycles using the following parameters: 95° C. 10 minutes; and 95° C. for 15 seconds/60° C. for 1 minute for a total of 40 cycles.

In the following sections, numerous Tables provide the sequences used for the primers and the probe of the invention, as well as the relative expression results which were obtained for the various cell cultures employed.

A. Clone 20468752

Table 22 and Table 23 provide primer sequence information and the relative expression results, respectively, for Clone 20468752. The relative expression results for Clone 20468752shown in Table 23 indicate relatively high expression in certain central nervous system tumors and melanomas, and suppression in most colon cancer, breast cancer, ovarian cancer, prostate cancer, lung cancer, and liver cancer samples, compared to the respective normal cell samples from the same tissues.

TABLE 22
Gene: 20468752 Probe Designation: Ag79

| | | Start |
|---|---|---|
| Primer/Probe | Sequence | Position |
| Forward | 5′-CAGTCAATGGGTACCAGAAAATAACA-3′ | 984 |
| | (SEQ ID NO:83) | |
| Probe | FAM-5′-CCTGGGCTTATCAACGGACGCCA-3′-TAMRA | 1016 |
| | (SEQ ID NO:84) | |
| Reverse | 5′-ACCACGGTGCCAATTTTAGC-3′ | 1040 |
| | (SEQ ID NO:85) | |

TABLE 23
Relative Expression

| Tissue Name | Relative Expression, % |
|---|---|
| Endothelial cells | 0.03 |
| Endothelial cells (treated) | 0.02 |
| Pancreas | 4.94 |
| Pancreatic Ca. CAPAN 2 | 0.02 |
| Adipose | 1.61 |
| Adrenal gland | 17.42 |
| Thyroid | 16.71 |
| Salivary gland | 2.58 |
| Pituitary gland | 60.34 |
| Brain (fetal) | 0.91 |
| Brain (whole) | 15.15 |
| Brain (amygdala) | 14.65 |
| Brain (cerebellum) | 5.25 |
| Brain (hippocampus) | 41.64 |
| Brain (substantia nigra) | 15.74 |
| Brain (thalamus) | 13.93 |
| Brain (hypothalamus) | 18.06 |
| Spinal cord | 41.82 |
| CNS ca. (glio/astro) U87-MG | 79.68 |
| CNS ca. (glio/astro) U-118-MG | 0.45 |
| CNS ca. (astro) SW1783 | 12.95 |
| CNS ca.* (neuro; met) SK-N-AS | 0.01 |
| CNS ca (astro) SF-539 | 0.26 |
| CNS ca. (astro) SNB-75 | 0 |
| CNS ca. (glio) SNB-19 | 0.44 |
| CNS ca. (glio) U251 | 0.23 |
| CNS ca. (glio) SF-295 | 15.48 |
| Heart | 28.98 |
| Skeletal muscle | 6.05 |
| Bone marrow | 2.62 |
| Thymus | 8.46 |
| Spleen | 11.5 |
| Lymph node | 3.06 |
| Colon (ascending) | 2.14 |
| Stomach | 10.43 |
| Small intestine | 58.02 |
| Colon ca. SW480 | 0.02 |
| Colon ca.* (SW480 met)SW620 | 0.02 |
| Colon ca. HT29 | 0.16 |
| Colon ca. HCT-116 | 0.04 |
| Colon ca. CaCo-2 | 15.17 |
| Colon ca. HCT-15 | 0.16 |
| Colon ca. HCC-2998 | 0.05 |
| Gastric ca.* (liver met) NCI-N87 | 0.04 |
| Bladder | 9.56 |
| Trachea | 14.44 |
| Kidney | 6.71 |
| Kidney (fetal) | 11.86 |
| Renal ca. 786-0 | 0.45 |
| Renal ca. A498 | 0.23 |
| Renal ca. RXF 393 | 0.67 |
| Renal ca. ACHN | 2.34 |
| Renal ca. UO-31 | 1.17 |
| Renal ca. TK-10 | 0.01 |
| Liver | 24.48 |
| Liver (fetal) | 8.03 |
| Liver ca. (hepatoblast) HepG2 | 0.02 |
| Lung | 0.39 |
| Lung (fetal) | 12.41 |
| Lung ca. (small cell) LX-1 | 0.09 |
| Lung ca. (small cell) NCI-H69 | 6.25 |
| Lung ca. (s.cell var.) SHP-77 | 0.02 |
| Lung ca. (large cell) NCI-H460 | 0.02 |
| Lung ca. (non-sm. cell) A549 | 0.54 |
| Lung ca. (non-s.cell) NCI-H23 | 0.16 |
| Lung Ca (non-s.cell) HOP-62 | 1.24 |
| Lung ca. (non-s.cl) NCI-H522 | 0.01 |
| Lung ca. (squam.) SW 900 | 0.17 |
| Lung ca. (squam.) NCI-H596 | 1.98 |
| Mammary gland | 11.42 |
| Breast ca.* (pl. effusion) MCF-7 | 0 |
| Breast ca.* (pl.ef) MDA-MB-231 | 0.02 |
| Breast ca.* (pl. effusion) T47D | 2.34 |
| Breast ca. BT-549 | 0.03 |
| Breast ca. MDA-N | 0.23 |
| Ovary | 16.48 |
| Ovarian ca. OVCAR-3 | 0.72 |
| Ovarian ca. OVCAR-4 | 1.10 |
| Ovarian ca. OVCAR-5 | 0.37 |
| Ovarian ca. OVCAR-8 | 0.26 |
| Ovarian ca. IGROV-1 | 0.10 |
| Ovarian ca.* (ascites) SK-OV-3 | 0.01 |
| Uterus | 5.33 |
| Placenta | 100 |
| Prostate | 12.32 |
| Prostate ca.* (bone met)PC-3 | 0.03 |
| Testis | 1.24 |
| Melanoma Hs688(A).T | 55.36 |
| Melanomas (met) Hs688(B).T | 48.07 |
| Melanoma UACC-62 | 0.03 |
| Melanoma M14 | 0.59 |
| Melanoma LOX IMVI | 0.16 |
| Melanoma* (met) SK-MEL-5 | 0.02 |
| Melanoma SK-MEL-28 | 0.13 |

Table 24 and Table 25 provide primer sequence information and the relative expression results, respectively, for Clone 11692010.0.51. As is shown in Table 25, high levels of expression, relative to normal cells, is found in certain ovarian cancer cell lines, in gastric cancer, and a colon cancer cell line. In addition, the protein encoded by this clone is also broadly expressed in lung cancers and certain CNS cancer cells.

TABLE 24
Gene: 11692010 Probe Designation: Ag92

| Primer/Probe | Sequence | Start Position |
|---|---|---|
| Forward | 5′-GCTAAATCCTGTCCATCTGTGT-3′ | 538 |
| | (SEQ ID NO:86) | |
| Probe | TET-5′-TGAAACCCGCATCGCAGCGA-3′-TAMRA | |
| | (SEQ ID NO:87) | |
| Reverse | 5′-ATGGATGTCAGAAAGCGATCA-3′ | 592 |
| | (SEQ ID NO:88) | |

TABLE 25
Relative Expression

| Tissue Name | Relative Expression, % |
|---|---|
| Endothelial cells | 0.03 |
| Endothelial cells (treated) | 0.1 |
| Pancreas | 1.63 |
| Pancreatic ca. CAPAN 2 | 3.26 |
| Adipose | 8.54 |
| Adrenal gland | 0.91 |
| Thyroid | 4.12 |
| Salivary gland | 0.19 |
| Pituitary gland | 0.57 |
| Brain (fetal) | 2.57 |
| Brain (whole) | 16.27 |
| Brain (amygdala) | 0.4 |
| Brain (cerebellum) | 100 |
| Brain (hippocampus) | 12.16 |
| Brain (substantia nigra) | 0.17 |
| Brain (thalamus) | 2.88 |
| Brain (hypothalamus) | 1.5 |
| Spinal cord | 1.10 |
| CNS ca. (glio/astro) U87-MG | 0.10 |
| CNS ca. (glio/astro) U-118-MG | 0.08 |
| CNS ca. (astro) SW1783 | 0.22 |
| CNS ca.* (neuro; met) SK-N-AS | 1.13 |
| CNS ca. (astro) SF-539 | 0 |
| CNS ca. (astro) SNB-75 | 9.47 |
| CNS ca. (glio) SNB-19 | 4.36 |
| CNS ca. (glio) U251 | 0 |
| CNS ca. (glio) SF-295 | 0 |
| Heart | 0.48 |
| Skeletal muscle | 2.22 |
| Bone marrow | 0 |
| Thymus | 13.77 |
| Spleen | 0.03 |
| Lymph node | 0.15 |
| Colon (ascending) | 3.42 |
| Stomach | 13.12 |
| Small intestine | 1.23 |
| Colon ca. SW480 | 0.06 |
| Colon ca.* (SW480 met)SW620 | 0 |
| Colon ca. HT29 | 1.00 |
| Colon ca. HCT-116 | 0 |
| Colon ca. CaCo-2 | 20.88 |
| Colon ca. HCT-15 | 0.77 |
| Colon ca. HCC-2998 | 0.4 |
| Gastric ca.* (liver met) NCI-N87 | 19.89 |
| Bladder | 1.95 |
| Trachea | 4.54 |
| Kidney | 7.75 |
| Kidney (fetal) | 20.73 |
| Renal ca. 786-0 | 0.45 |
| Renal ca. A498 | 0.39 |
| Renal ca. RXF 393 | 0.37 |
| Renal ca. ACHN | 0.91 |
| Renal ca. UO-31 | 0.77 |
| Renal ca. TK-10 | 7.80 |
| Liver | 2.59 |
| Liver (fetal) | 2.9 |
| Liver ca. (hepatoblast) HepG2 | 0 |
| Lung | 3.10 |
| Lung (fetal) | 10.73 |
| Lung ca. (small cell) LX-1 | 0.95 |
| Lung ca. (small cell) NCI-H69 | 5.26 |
| Lung ca. (s.cell var.) SHP-77 | 0 |
| Lung ca. (large cell)NCI-H460 | 0 |
| Lung ca. (non-sm. Cell) A549 | 5.79 |
| Lung ca. (non-s.cell) NCI-H23 | 0.3 |
| Lung ca. (non-s.cell) HOP-62 | 2.74 |
| Lung ca. (non-s.cl) NCI-H522 | 1.63 |
| Lung ca. (squam.) SW 900 | 4.27 |
| Lung ca. (squam.) NCI-H596 | 6 |
| Mammary gland | 2.54 |
| Breast ca.* (pl. effusion) MCF-7 | 4.45 |
| Breast ca.* (pl.ef) MDA-MB-231 | 0 |
| Breast ca.* (pl. effusion) T47D | 0.09 |
| Breast ca. BT-549 | 0 |
| Breast ca. MDA-N | 1.46 |
| Ovary | 0.86 |
| Ovarian ca. OVCAR-3 | 0.85 |
| Ovarian ca. OVCAR-4 | 0.55 |
| Ovarian ca. OVCAR-5 | 16.27 |
| Ovarian ca. OVCAR-8 | 0.59 |
| Ovarian ca. IGROV-1 | 6.93 |
| Ovarian ca.* (ascites) SK-OV-3 | 2.76 |
| Uterus | 10.15 |
| Placenta | 1.6 |
| Prostate | 6.38 |
| Prostate ca.* (bone met)PC-3 | 0 |
| Testis | 22.22 |
| Melanoma Hs688(A).T | 0.22 |
| Melanoma* (met) Hs688(B).T | 0.15 |
| Melanoma UACC-62 | 1.26 |
| Melanoma M14 | 1.30 |
| Melanoma LOX IMVI | 0.08 |
| Melanoma* (met) SK-MEL-5 | 0.64 |
| Melanoma SK-MEL-28 | 0.96 |

C. Clone 27835981.0.1

Table 26 and Table 27 provide primer sequence information and the relative expression results, respectively, for Clone 27835981.0.1. The relative expression level for Clone 27835981.0.1, as shown in Table 27, indicate that the protein encoded by this clone is over-expressed, relative to the respective normal cell lines for the same tissues, in virtually all cancer cell lines examined.

TABLE 26
Gene: 27835981 Probe Designation: Ag99

| | | |
|---|---|---|
| Primer/Probe | Sequence | Start Position |

| | | |
|---|---|---|
| Forward 5′CAGTCACACAGCTGCTCTATTCTCA-3′ | 820 | |
| | (SEQ ID NO:99) | |
| Probe | FAM-5′AAATCTACCCCTTGCGTGGCTGGAAC-3′-TAMRA | 848 |
| | (SEQ ID NO:100) | |
| Reverse | 5′-GGACACCTCCAGGGAAACGT-3′ | 876 |
| | (SEQ ID NO:101) | |

TABLE 27
Relative Expression Levels

| Tissue Name | Relative Expression, % |
|---|---|
| Endothelial cells | 78.31 |
| Endothelial cells (treated) | 47.36 |
| Pancreas | 6.92 |
| Pancreatic ca. CAPAN 2 | 47.36 |
| Adipose | 0.75 |
| Adrenal gland | 5.14 |
| Thyroid | 9.26 |
| Salivary gland | 6.92 |
| Pituitary gland | 0 |
| Brain (fetal) | 6.92 |
| Brain (whole) | 2.02 |
| Brain (amygdala) | 5.14 |
| Brain (cerebellum) | 1.46 |
| Brain (hippocampus) | 3.79 |
| Brain (substantia nigra) | 5.14 |
| Brain (thalamus) | 5.14 |
| Brain (hypothalamus) | 5.14 |
| Spinal cord | 6.92 |
| CNS ca. (glio/astro) U87-MG | 36.56 |
| CNS ca. (glio/astro) U-118-MG | 36.56 |
| CNS ca. (astro) SW1783 | 47.36 |
| CNS ca.* (neuro; met) SK-N-AS | 36.56 |
| CNS ca. (astro) SF-539 | 36.56 |
| CNS ca. (astro) SNB-75 | 21.45 |
| CNS ca. (glio) SNB-19 | 0 |
| CNS ca. (glio) U251 | 28.08 |
| CNS ca. (glio) SF-295 | 21.45 |
| Heart | 12.32 |
| Skeletal muscle | 28.08 |
| Bone marrow | 21.45 |
| Thymus | 5.14 |
| Spleen | 12.32 |
| Lymph node | 9.26 |
| Colon (ascending) | 3.79 |
| Stomach | 5.14 |
| Small intestine | 9.26 |
| Colon ca. SW480 | 61.04 |
| Colon ca.* (SW480 met)SW620 | 61.04 |
| Colon ca. HT29 | 47.36 |
| Colon ca. HCT-116 | 100 |
| Colon ca. CaCo-2 | 21.45 |
| Colon ca. HCT-15 | 0 |
| Colon ca. HCC-2998 | 36.56 |
| Gastric ca.* (liver met) NCI-N87 | 12.32 |
| Bladder | 9.26 |
| Trachea | 5.14 |
| Kidney | 6.92 |
| Kidney (fetal) | 2.78 |
| Renal ca. 786-0 | 28.08 |
| Renal ca. A498 | 21.45 |
| Renal ca. RXF 393 | 36.56 |
| Renal ca. ACHN | 47.36 |
| Renal ca. UO-31 | 28.08 |
| Renal ca. TK-10 | 28.08 |
| Liver | 9.26 |
| Liver (fetal) | 0 |
| Liver ca. (hepatoblast) HepG2 | 47.36 |
| Lung | 0 |
| Lung (fetal) | 6.92 |
| Lung ca. (small cell) LX-1 | 36.56 |
| Lung ca. (small cell) NCI-H69 | 9.26 |
| Lung ca. (s.cell var.) SHP-77 | 61.04 |
| Lung ca. (large cell)NCI-H460 | 61.04 |
| Lung ca. (non-sm. cell) A549 | 21.45 |
| Lung ca. (non-s.cell) NCI-H23 | 28.08 |
| Lung ca. (non-s.cell) HOP-62 | 28.08 |
| Lung ca. (non-s.cl) NCI-H522 | 28.08 |
| Lung ca. (squam.) SW900 | 9.26 |
| Lung ca. (squam.) NCI-H596 | 12.32 |
| Mammary gland | 2.02 |
| Breast ca.* (pl. effusion) MCF-7 | 21.45 |
| Breast ca.* (pl.ef) MDA-MB-231 | 61.04 |
| Breast ca.* (pl. effusion) T47D | 9.26 |
| Breast ca. BT-549 | 78.31 |
| Breast ca. MDA-N | 28.08 |
| Ovary | 9.26 |
| Ovarian ca. OVCAR-3 | 28.08 |
| Ovarian ca. OVCAR-4 | 36.56 |
| Ovarian ca. OVCAR-5 | 6.92 |
| Ovarian ca. OVCAR-8 | 0 |
| Ovarian ca. IGROV-1 | 36.56 |
| Ovarian ca.* (ascites) SK-OV-3 | 28.08 |
| Uterus | 5.14 |
| Placenta | 6.92 |
| Prostate | 5.14 |
| Prostate ca.* (bone met)PC-3 | 78.31 |
| Testis | 0.75 |
| Melanoma Hs688(A).T | 47.36 |
| Melanoma* (met) Hs688(B).T | 28.08 |
| Melanoma UACC-62 | 78.31 |
| Melanoma M14 | 0 |
| Melanoma LOX IMVI | 21.45 |
| Melanoma* (met) SK-MEL-5 | 47.36 |
| Melanoma SK-MEL-28 | 21.45 |

D. Clone 21399247.0.1

Table 28 provides primer sequence information for Clone 21399247.0.1. The expression analysis for Clone 21399247.0.1 was replicated a total of six-times. The protein encoded by the clone is broadly expressed in most of the tissues examined (i.e., the same cell lines as in the other Tables included in this section of the Specific Example). Furthermore, the encoded protein is also particularly strongly expressed in certain cancers, such as melanoma, prostate cancer, lung cancer, and colon cancer.

TABLE 28
Gene: 21399247 Probe Designation: Ag109

| Primer/Probe | Sequence | Start Position |
|---|---|---|
| Forward | 5′-CCTGCAAAGCCGTGAGGT-3′ | 1547 |
| | (SEQ ID NO:102) | |
| Probe | FAM-5′-ACGGCATCTCTGTTGCCGGAACC-3′-TAMRA | 1568 |
| | (SEQ ID NO:103) | |
| Reverse | 5′-GGTGTCCTGGTAGATTCGGAAG 3′ | 1601 |
| | (SEQ ID NO:104) | |

E. Clone 17132296

Table 29 and Table 30 provide primer sequence information and the relative expression results, respectively, for Clone 17132296. The expression analysis for Clone 17132296, shown in Table 30, demonstrates that the protein encoded by this clone is over-expressed, relative to normal tissue cell lines, in ovarian cancer, breast cancer, and colon cancer.

TABLE 29
Gene: 17132296 Probe Designation: Ag162

| Primer/Probe | Sequence |
|---|---|
| Forward | 5′-GTACTGCCGCCAGCTTACCT-3′ |
| | (SEQ ID NO:105) |
| Probe | TET-5′CACAGAGCCAGCAGTGACACATGACAAA-3′-TAMRA |
| | (SEQ ID NO:106) |
| Reverse | 5′-GACATGGCTTTCGTAAATAATGCA-3′ |
| | (SEQ ID NO:107) |

TABLE 30
Relative Expression Levels

| Tissue Name | Relative Expression, % |
|---|---|
| Endothelial cells | 0.04 |
| Endothelial cells (treated) | 3.12 |
| Pancreas | 1.4 |
| Pancreatic ca. CAPAN 2 | 0 |
| Adipose | 1.21 |
| Adrenal gland | 2.55 |
| Thyroid | 3.22 |
| Salivary gland | 1.60 |
| Pituitary gland | 3.83 |
| Brain (fetal) | 4 |
| Brain (whole) | 27.73 |
| Brain (amygdala) | 11.87 |
| Brain (cerebellum) | 41.5 |
| Brain (hippocampus) | 11.55 |
| Brain (substantia nigra) | 22.40 |
| Brain (thalamus) | 21.92 |
| Brain (hypothalamus) | 2.04 |
| Spinal cord | 15.21 |
| CNS ca. (glio/astro) U87-MG | 23.57 |
| CNS ca. (glio/astro) U-118-MG | 0.01 |
| CNS ca. (astro) SW1783 | 0.66 |
| CNS ca.* (neuro; met) SK-N-AS | 18.37 |
| CNS ca. (astro) SF-539 | 0 |
| CNS ca. (astro) SNB-75 | 21.94 |
| CNS ca. (glio) SNB-19 | 46.12 |
| CNS ca. (glio) U251 | 8.94 |
| CNS ca. (glio) SF-295 | 0 |
| Heart | 2.97 |
| Skeletal muscle | 6.45 |
| Bone marrow | 5.19 |
| Thymus | 1.35 |
| Spleen | 0.9 |
| Lymph node | 2.81 |
| Colon (ascending) | 1.39 |
| Stomach | 2.22 |
| Small intestine | 1.54 |
| Colon ca. SW480 | 1.20 |
| Colon ca.* (SW480 met)SW620 | 0 |
| Colon ca. HT29 | 0 |
| Colon ca. HCT-116 | 0.01 |
| Colon ca. CaCo-2 | 2.51 |
| Colon ca. HCT-15 | 10.11 |
| Colon ca. HCC-2998 | 17.88 |
| Gastric ca.* (liver met) NCI-N87 | 5.16 |
| Bladder | 12.24 |
| Trachea | 1.84 |
| Kidney | 27.13 |
| Kidney (fetal) | 4.71 |
| Renal ca. 786-0 | 8.94 |
| Renal ca. A498 | 10.6 |
| Renal ca. RXF 393 | 2.25 |
| Renal ca. ACHN | 7.53 |
| Renal ca. UO-31 | 0.86 |
| Renal ca. TK-10 | 10.03 |
| Liver | 15.14 |
| Liver (fetal) | 4.52 |
| Liver ca. (hepatoblast) HepG2 | 0.02 |
| Lung | 4.91 |
| Lung (fetal) | 1.04 |
| Lung ca. (small cell) LX-1 | 0.22 |
| Lung ca. (small cell) NCI-H69 | 3.26 |
| Lung ca. (s.cell var.) SHP-77 | 0 |
| Lung ca. (large cell)NCI-H460 | 0 |
| Lung ca. (non-sm. cell) A549 | 3.21 |
| Lung ca. (non-s.cell) NCI-H23 | 12.72 |
| Lung ca. (non-s.cell) HOP-62 | 0.5 |
| Lung ca. (non-s.d) NCI-H522 | 15.49 |
| Lung ca. (squam.) SW 900 | 1.11 |
| Lung ca. (squam.)NCI-H596 | 1.33 |
| Mammary gland | 3.85 |
| Breast ca* (pl. effusion) MCF-7 | 17.73 |
| Breast ca.* (pl.ef) MDA-MB-231 | 2.49 |
| Breast ca.* (pl. effusion) T47D | 4.21 |
| Breast ca. BT-549 | 0.01 |
| Breast ca. MDA-N | 27.8 |
| Ovary | 1.15 |
| Ovarian ca. OVCAR-3 | 1.9 |
| Ovarian ca. OVCAR-4 | 2.81 |
| Ovarian ca. OVCAR-5 | 1.15 |
| Ovarian ca. OVCAR-8 | 8.27 |
| Ovarian ca. IGROV-1 | 4.47 |
| Ovarian ca.* (ascites) SK-OV-3 | 97.72 |
| Uterus | 24.78 |
| Placenta | 8.9 |
| Prostate | 1.99 |
| Prostate ca.* (bone met)PC-3 | 0.01 |
| Testis | 100 |
| Melanoma Hs688(A).T | 16.52 |
| Melanoma* (met) Hs688(B).T | 13.53 |
| Melanoma UACC-62 | 14.35 |
| Melanoma M14 | 12.97 |
| Melanoma LOX IMVI | 10.82 |
| Melanoma* (met) SK-MEL-5 | 24.65 |
| Melanoma SK-MEL-28 | 21.34 |

F. Clone 17931354

Table 31 and Table 32 provide primer sequence information and the relative expression results, respectively, for Clone 17931354. The expression analysis results for Clone 17931354 are shown in Table 32. Interestingly, the protein encoded by this clone is prominently detected in two lung cancer cell lines, but not within normal lung cells.

TABLE 31
Gene: 17931354 Probe Name: Ag124

| Primer/Probe | Sequence | Start Position |
|---|---|---|
| Forward | 5′-CGCCGCTACAACCGCAT-3′ | 3070 |
| | (SEQ ID NO:108) | |
| Probe | FAM-5′-CCATAGAGTCAGCGTTTGACAATCCAACTTACG-3′-TAMRA | 3089 |
| | (SEQ ID NO:109) | |
| Reverse | 5′-CTGCAAAGGAAAGAGATCCAGTC-3′ | 3123 |
| | (SEQ ID NO:110) | |

TABLE 32
Relative Expression Levels

| Tissue Name | Relative Expression, % |
|---|---|
| Endothelial cells | 0.11 |
| Endothelial cells (treated) | 0.07 |
| Pancreas | 0.19 |
| Pancreatic ca. CAPAN 2 | 0.07 |
| Adipose | 0 |
| Adrenal gland | 0 |
| Thyroid | 0.01 |
| Salivary gland | 0 |
| Pituitary gland | 0 |
| Brain (fetal) | 46.93 |
| Brain (whole) | 18.64 |
| Brain (amygdala) | 39.47 |
| Brain (cerebellum) | 70.04 |
| Brain (hippocampus) | 26 |
| Brain (substantia nigra) | 11.08 |
| Brain (thalamus) | 29.78 |
| Brain (hypothalamus) | 12.08 |
| Spinal cord | 3.02 |
| CNS ca. (glio/astro) U87-MG | 0.05 |
| CNS ca. (glio/astro) U-118-MG | 0.05 |
| CNS ca. (astro) SW1783 | 0.07 |
| CNS ca.* (neuro; met) SK-N-AS | 0.05 |
| CNS ca. (astro) SF-539 | 0.05 |
| CNS ca. (astro) SNB-75 | 0.03 |
| CNS ca. (glio) SNB-19 | 7.12 |
| CNS ca. (glio) U251 | 2.65 |
| CNS ca. (glio) SF-295 | 0.03 |
| Heart | 0.02 |
| Skeletal muscle | 0.04 |
| Bone marrow | 0.03 |
| Thymus | 0 |
| Spleen | 0.02 |
| Lymph node | 0.01 |
| Colon (ascending) | 0 |
| Stomach | 0 |
| Small intestine | 1.00 |
| Colon ca. SW480 | 0.08 |
| Colon ca.* (SW480 met)SW620 | 0.08 |
| Colon ca. HT29 | 0.07 |
| Colon ca. HCT-116 | 0.14 |
| Colon ca. CaCo-2 | 0.03 |
| Colon ca. HCT-15 | 0 |
| Colon ca. HCC-2998 | 0.05 |
| Gastric ca.* (liver met) NCI-N87 | 0.02 |
| Bladder | 0.01 |
| Trachea | 0.02 |
| Kidney | 0 |
| Kidney (fetal) | 0 |
| Renal ca. 786-0 | 0.04 |
| Renal ca. A498 | 0.03 |
| Renal ca. RXF 393 | 0.05 |
| Renal ca. ACHN | 0.07 |
| Renal ca. UO-31 | 0.04 |
| Renal ca. TK-10 | 0.04 |
| Liver | 0.01 |
| Liver (fetal) | 0 |
| Liver ca. (hepatoblast) HepG2 | 0.07 |
| Lung | 0 |
| Lung (fetal) | 0 |
| Lung ca. (small cell) LX-1 | 0.05 |
| Lung ca. (small cell) NCI-H69 | 100 |
| Lung ca. (s.cell var.) SHP-77 | 0.08 |
| Lung ca. (large cell)NCI-H460 | 0.08 |
| Lung ca. (non-sm. cell) A549 | 0.03 |
| Lung ca. (non-s.cell) NCI-H23 | 0.04 |
| Lung Ca (non-s.cell) HOP-62 | 0.04 |
| Lung ca. (non-s.d) NCI-H522 | 0.04 |
| Lung ca. (squam.) SW 900 | 0.01 |
| Lung ca. (squam.) NCI-H596 | 74.46 |
| Mammary gland | 0 |
| Breast ca.* (pl. effusion) MCF-7 | 0.03 |
| Breast ca.* (pl.ef) MDA-MB-231 | 0.08 |
| Breast ca.* (pl. effusion) T47D | 0.01 |
| Breast ca. BT-549 | 0.11 |
| Breast ca. MDA-N | 0.04 |
| Ovary | 0.01 |
| Ovarian ca. OVCAR-3 | 0.04 |
| Ovarian ca. OVCAR-4 | 0.05 |
| Ovarian ca. OVCAR-5 | 0 |
| Ovarian ca. OVCAR-8 | 0 |
| Ovarian ca. IGROV-1 | 0.05 |
| Ovarian ca* (ascites) SK-OV-3 | 0.04 |
| Uterus | 0 |
| Placenta | 0.07 |
| Prostate | 0 |
| Prostate ca.* (bone met)PC-3 | 0.11 |
| Testis | 0.28 |
| Melanoma Hs688(A).T | 0.07 |
| Melanoma* (met) Hs688(B).T | 0.04 |
| Melanoma UACC-62 | 0.11 |
| Melanoma M14 | 0 |
| Melanoma LOX IMVI | 0.03 |
| Melanoma* (met) SK-MEL-5 | 0.07 |
| Melanoma SK-MEL-28 | 0.03 |

G. Clone 7520500

Table 33 and Table 34 provide primer sequence information and the relative expression results, respectively, for Clone 7520500. The expression analysis results for the protein encoded by Clone7520500 are shown in Table 34. As was found with Clone 17931354, the protein encoded by this Clone 7520500 is prominently detected in two lung cancer cell lines, but not within normal lung cells.

TABLE 33
Gene: 7520500
Probe Designation: Ag90

| | | Start |
|---|---|---|
| Primer/Probe | Sequence | Position |
| Forward | 5′-TTGGCCTGGACTGCTTCTTC-3′ | 977 |
| | (SEQ ID NO:111) | |
| Probe | TET-5′ | 999 |
| | CATCTCTGTCTACCCTGGCTATGGCGTG-3′- | |
| | TAMRA | |
| | (SEQ ID NO:112) | |
| Reverse | 5′-AGGCTGATATTCTGGACCTTGATT-3′ | 1029 |
| | (SEQ ID NO:113) | |

TABLE 34
Relative Expression Levels

| Tissue Name | Relative Expression, % |
|---|---|
| Endothelial cells | 0.07 |
| Endothelial cells (treated) | 0.04 |
| Pancreas | 0.22 |
| Pancreatic ca. CAPAN 2 | 0.04 |
| Adipose | 0 |
| Adrenal gland | 0 |
| Thyroid | 0 |
| Salivary gland | 0 |
| Pituitary gland | 0 |
| Brain (fetal) | 90.94 |
| Brain (whole) | 23.47 |
| Brain (amygdala) | 49.07 |
| Brain (cerebellum) | 95.02 |
| Brain (hippocampus) | 47.9 |
| Brain (substantia nigra) | 14.28 |
| Brain (thalamus) | 26.37 |
| Brain (hypothalamus) | 14.59 |
| Spinal cord | 3.05 |
| CNS ca. (glio/astro) U87-MG | 0.03 |
| CNS ca. (glio/astro) U-118-MG | 0.03 |
| CNS ca. (astro) SW1783 | 0.04 |
| CNS ca.* (neuro; met) SK-N-AS | 3.11 |
| CNS ca. (astro) SF-539 | 0.03 |
| CNS ca. (astro) SNB-75 | 0.02 |
| CNS ca. (glio) SNB-19 | 7.73 |
| CNS ca. (glio) U251 | 2.83 |
| CNS ca. (glio) SF-295 | 0.02 |
| Heart | 0.01 |
| Skeletal muscle | 0.03 |
| Bone marrow | 0.02 |
| Thymus | 0.12 |
| Spleen | 0.01 |
| Lymph node | 0 |
| Colon (ascending) | 0.07 |
| Stomach | 0.2 |
| Small intestine | 0.9 |
| Colon ca. SW480 | 0.57 |
| Colon ca.* (SW480 met)SW620 | 0.06 |
| Colon ca. HT29 | 0.04 |
| Colon ca. HCT-116 | 0.09 |
| Colon ca. CaCo-2 | 0.02 |
| Colon ca. HCT-15 | 0 |
| Colon ca. HCC-2998 | 0.03 |
| Gastric ca.* (liver met) NCI-N87 | 0.01 |
| Bladder | 0 |
| Trachea | 0.07 |
| Kidney | 0 |
| Kidney (fetal) | 0 |
| Renal ca. 786-0 | 0.03 |
| Renal ca. A498 | 0.02 |
| Renal ca. RXF 393 | 0.03 |
| Renal ca. ACHN | 0.04 |
| Renal ca. UO-31 | 0.03 |
| Renal ca. TK-10 | 0.03 |
| Liver | 0 |
| Liver (fetal) | 0 |
| Liver ca. (hepatoblast) HepG2 | 0.04 |
| Lung | 0 |
| Lung (fetal) | 0 |
| Lung ca. (small Cell) LX-1 | 0.21 |
| Lung ca. (small cell) NCI-H69 | 100 |
| Lung ca. (s.cell var.) SHP-77 | 0.06 |
| Lung ca. (large cell)NCI-H460 | 0.06 |
| Lung ca. (non-sm. cell) A549 | 0.02 |
| Lung ca. (non-s.cell) NCI-H23 | 0.03 |
| Lung ca. (non-s.cell) HOP-62 | 0.03 |
| Lung ca. (non-s.d) NCI-H522 | 0.03 |
| Lung ca. (squam.) SW 900 | 0 |
| Lung ca. (squarn.)NCI-H596 | 71.61 |
| Mammary gland | 0.04 |
| Breast ca.* (pl. effusion) MCF-7 | 0.02 |
| Breast ca.* (pl.ef) MDA-MB-231 | 0.06 |
| Breast ca.* (pl. effusion) T47D | 0 |
| Breast ca. BT-549 | 0.07 |
| Breast ca. MDA-N | 0.03 |
| Ovary | 0 |
| Ovarian ca. OVCAR-3 | 0.03 |
| Ovarian ca. OVCAR-4 | 0.03 |
| Ovarian ca. OVCAR-5 | 0 |
| Ovarian ca. OVCAR-8 | 0 |
| Ovarian ca. IGROV-1 | 0.03 |
| Ovarian ca.* (ascites) SK-OV-3 | 0.07 |
| Uterus | 0.02 |
| Placenta | 0.02 |
| Prostate | 0 |
| Prostate ca.* (bone met)PC-3 | 0.07 |
| Testis | 0.49 |
| Melanoma Hs688(A).T | 0.04 |
| Melanoma* (met) Hs688(B).T | 0.03 |
| Melanoma UACC-62 | 0.07 |
| Melanoma M14 | 0 |
| Melanoma LOX IMVI | 0.02 |
| Melanoma* (met) SK-MEL-5 | 0.05 |
| Melanoma SK-MEL-28 | 0.02 |

H. Clone 17941787

Table 35 and Table 36 provide primer sequence information and the relative expression results, respectively, for Clone 17941787. The expression analysis results for Clone 17941787 are shown for a total of two trials in Table 36. From these results, it is seen that, relative to cells from normal tissues, prostate cancer, ovarian cancer, breast cancer, lung cancer, renal cancer, CNS cancer and pancreatic cancer cell lines over-express the protein encoded by this clone to extremely high levels.

TABLE 35
Gene: 17941787
Probe Designation: Ag96

| | | Start |
|---|---|---|
| Primer/Probe | Sequence | Position |
| Forward | 5′-CCAAGTAGATGGGTTCTGTTTGC-3′ | 1169 |
| | (SEQ ID NO:114) | |
| Probe | FAM-5′ | 1194 |
| | CCCAGTTACCTCCACAGGGTATTTCCCA-3′- | |
| | TAMRA | |
| | (SEQ ID NO:115) | |
| Reverse | 5′-CGACGCTGCTGCTCAGTATAAC-3′ | 1282 |
| | (SEQ ID NO:116) | |

TABLE 36
Relative Expression Levels

| | Rel. Expr. (%) | Rel. Expr. (%) |
|---|---|---|
| Tissue Name | tm256f | tm341f |
| Endothelial cells | 17.05 | 2.44 |
| Endothelial cells (treated) | 18.41 | 8.66 |
| Pancreas | 2.11 | 0.72 |
| Pancreatic ca. CAPAN 2 | 24.36 | 9.32 |
| Adipose | 0.96 | 0.53 |
| Adrenal gland | 6.14 | 3.10 |
| Thyroid | 3.17 | 3.01 |
| Salivary gland | 1.88 | 4.32 |
| Pituitary gland | 10.32 | 8.02 |
| Brain (fetal) | 17.02 | 14.67 |
| Brain (whole) | 16.03 | 7.72 |
| Brain (amygdala) | 11.84 | 9.91 |
| Brain (cerebellum) | 40.7 | 4.52 |
| Brain (hippocampus) | 32.22 | 8.09 |
| Brain (substantia nigra) | 5.2 | 6.71 |
| Brain (thalamus) | 7.40 | 4.38 |
| Brain (hypothalamus) | 13.29 | 14.33 |
| Spinal cord | 2.64 | 0.79 |
| CNS ca. (glio/astro) U87-MG | 30.88 | 20.08 |
| CNS ca. (glio/astro) U-118-MG | 22.97 | 19.29 |
| CNS ca. (astro) SW1783 | 38.58 | 21.16 |
| CNS ca.* (neuro; met) SK-N-AS | 36.05 | 19.95 |
| CNS ca. (astro) SF-539 | 51.50 | 34.64 |
| CNS ca. (astro) SNB-75 | 53.55 | 38.64 |
| CNS ca. (glio) SNB-19 | 12.18 | 8.24 |
| CNS ca. (glio) U251 | 11.19 | 2.86 |
| CNS ca. (glio) SF-295 | 19.53 | 15.51 |
| Heart | 16.96 | 16.47 |
| Skeletal muscle | 12.06 | 11.6 |
| Bone marrow | 2.46 | 1.28 |
| Thymus | 32.30 | 26.66 |
| Spleen | 3.34 | 3.01 |
| Lymph node | 2.83 | 0.84 |
| Colon (ascending) | 2.94 | 1.77 |
| Stomach | 3.37 | 4.68 |
| Small intestine | 2.54 | 1.16 |
| Colon ca. SW480 | 6.89 | 2.15 |
| Colon ca.* (SW480 met)SW620 | 5.33 | 2.15 |
| Colon ca. HT29 | 2.54 | 1.9 |
| Colon ca. HCT-116 | 0.12 | 2.75 |
| Colon ca. CaCo-2 | 1.42 | 1.28 |
| Colon ca. HCT-15 | 5.27 | 6.12 |
| Colon ca. HCC-2998 | 9.64 | 3.51 |
| Gastric ca.* (liver met) NCI-N87 | 0.05 | 0.97 |
| Bladder | 4.61 | 15.56 |
| Trachea | 2.32 | 1.07 |
| Kidney | 3.02 | 2.22 |
| Kidney (fetal) | 7.09 | 7.79 |
| Renal ca. 786-0 | 60.36 | 54.60 |
| Renal ca. A498 | 56.19 | 55.98 |
| Renal ca. RXF 393 | 64.31 | 40.17 |
| Renal ca. ACHN | 26.56 | 10.79 |
| Renal ca. UO-31 | 40.15 | 34.17 |
| Renal ca. TK-10 | 29.97 | 29.62 |
| Liver | 2.85 | 0.84 |
| Liver (fetal) | 2.98 | 1.11 |
| Liver ca. (hepatoblast) HepG2 | 1.08 | 1.9 |
| Lung | 0.63 | 1.11 |
| Lung (fetal) | 5.12 | 5.17 |
| Lung ca. (small cell) LX-1 | 1.79 | 1.67 |
| Lung ca. (small cell) NCI-H69 | 15.89 | 9.41 |
| Lung ca. (s.cell var.) SHP-77 | 0.07 | 33.53 |
| Lung ca. (large cell)NCI-H460 | 0.07 | 89.67 |
| Lung ca. (non-sm. cell) A549 | 16.79 | 14.19 |
| Lung ca. (non-s.cell)NCI-H23 | 14.39 | 15.32 |
| Lung ca. (non-s.cell) HOP-62 | 29.37 | 34.17 |
| Lung ca. (non-s.d) NCI-H522 | 39.60 | 27.12 |
| Lung ca. (squam.) SW 900 | 19.37 | 11.97 |
| Lung ca. (squam.) NCI-H596 | 25.10 | 32.49 |
| Mammary gland | 45.51 | 2.4 |
| Breast ca.* (pl. effusion) MCF-7 | 4.40 | 1.28 |
| Breast ca.* (pl.ef) MDA-MB-231 | 30.44 | 17.22 |
| Breast ca.* (pl. effusion) T47D | 4.57 | 0.84 |
| Breast ca. BT-549 | 0.1 | 62.45 |
| Breast ca. MDA-N | 33.64 | 20.95 |
| Ovary | 3.10 | 0.84 |
| Ovarian ca. OVCAR-3 | 7.24 | 8.09 |
| Ovarian ca. OVCAR-4 | 9.01 | 2.8 |
| Ovarian ca. OVCAR-5 | 17.02 | 22.21 |
| Ovarian ca. OVCAR-8 | 25.23 | 17.55 |
| Ovarian ca. IGROV-1 | 6.61 | 1.67 |
| Ovarian ca.* (ascites) SK-OV-3 | 31.43 | 21.38 |
| Uterus | 2.19 | 3.82 |
| Placenta | 3.93 | 0.87 |
| Prostate | 2.45 | 4.29 |
| Prostate ca.* (bone met)PC-3 | 0.1 | 100 |
| Testis | 7.31 | 8.11 |
| Melanoma Hs688(A).T | 46.5 | 17.44 |
| Melanoma* (met) Hs688(B).T | 44.76 | 15.85 |
| Melanoma UACC-62 | 17.05 | 4.72 |
| Melanoma M14 | 35.18 | 16.49 |
| Melanoma LOX IMVI | 91.46 | 68.77 |
| Melanoma* (met) SK-MEL-5 | 56.41 | 17.56 |
| Melanoma SK-MEL-28 | 100 | 86.85 |

I. Clone 16467945

Table 37 and Table 38 provide primer sequence information and the relative expression results, respectively, for Clone 16467945. The tissue expression analysis for Clone 16467945 is shown in Table 38. The results indicate that the protein encoded by this clone is highly over-expressed in certain cell lines derived from breast cancer, ovarian cancer, renal cancer, and colon cancer. In addition, the encoded protein is found to be strongly suppressed in lung cancer cell lines, in comparison with normal lung cells.

TABLE 37
Gene: 16467945
Probe Designation: Ag94

| | | Start |
|---|---|---|
| Primer/Probe | Sequence | Position |
| Forward | 5′-CCACCTACAACCCCAGAAAGG-3′ | 1491 |
| | (SEQ ID NO:117) | |
| Probe | FAM-5′- | 1460 |
| | CAACCACCGGACTGACAACTATAGCACCAG-3′- | |
| | TAMRA | |
| | (SEQ ID NO:118) | |
| Reverse | 5′-TGTAATCCCTCCTGGAGGTGTAC-3′ | 1431 |
| | (SEQ ID NO:119) | |

TABLE 38
Relative Expression Levels

| | Relative |
|---|---|
| Tissue Name | Expression(%) |
| Endothelial cells | 0.03 |
| Endothelial cells (treated) | 0.07 |
| Pancreas | 14.47 |
| Pancreatic ca. CAPAN 2 | 0.52 |
| Adipose | 0.65 |
| Adrenal gland | 1.79 |
| Thyroid | 75.56 |
| Salivary gland | 2.06 |
| Pituitary gland | 4.64 |
| Brain (fetal) | 9.1 |
| Brain (whole) | 1.06 |
| Brain (amygdala) | 1.21 |
| Brain (cerebellum) | 0.2 |
| Brain (hippocampus) | 1.83 |
| Brain (substantia nigra) | 3.07 |
| Brain (thalamus) | 0.8 |
| Brain (hypothalamus) | 14.83 |
| Spianl cord | 3.7 |
| CNS ca. (glio/astro) U87-MG | 0.01 |
| CNS ca. (glio/astro) U-118-MG | 0.01 |
| CNS ca. (astro) SW1783 | 0.13 |
| CNS ca.* (neuro; met) SK-N-AS | 0.01 |
| CNS ca. (astro) SF-539 | 1.35 |
| CNS ca. (astro) SNB-75 | 0.27 |
| CNS ca. (glio) SNB-19 | 0.02 |
| CNS ca. (glio) U251 | 0.8 |
| CNS ca. (glio) SF-295 | 0.18 |
| Heart | 1.88 |
| Skeletal muscle | 1.67 |
| Bone marrow | 0.53 |
| Thymus | 6.75 |
| Spleen | 3.70 |
| Lymph node | 1.01 |
| Colon (ascending) | 2.94 |
| Stomach | 4.22 |
| Small intestine | 11.51 |
| Colon ca. SW480 | 0.24 |
| Colon ca.* (SW480 met)SW620 | 0.02 |
| Colon ca. HT29 | 13.33 |
| Colon ca. HCT-116 | 0.03 |
| Colon ca. CaCo-2 | 21.94 |
| Colon ca. HCT-15 | 18.32 |
| Colon ca. HCC-2998 | 5.13 |
| Gastric ca.* (liver met) NCI-N87 | 31.33 |
| Bladder | 1.33 |
| Trachea | 3.63 |
| Kidney | 12.37 |
| Kidney (fetal) | 19.3 |
| Renal ca. 786-0 | 0 |
| Renal ca A498 | 0.12 |
| Renal ca. RXF 393 | 15.07 |
| Renal ca. ACHN | 70.06 |
| Renal ca. UO-31 | 0.1 |
| Renal ca. TK-10 | 0 |
| Liver | 1.46 |
| Liver (fetal) | 1.11 |
| Liver ca. (hepatoblast) HepG2 | 10.01 |
| Lung | 21.48 |
| Lung (fetal) | 29.57 |
| Lung ca. (small cell) LX-1 | 3.23 |
| Lung ca. (small cell) NCI-H69 | 7.75 |
| Lung ca. (s.cell var.) SHP-77 | 0.02 |
| Lung ca. (large cell)NCI-H460 | 0.02 |
| Lung ca. (non-sm. cell) A549 | 1.16 |
| Lung ca. (non-s.cell) NCI-H23 | 2.16 |
| Lung ca (non-s.cell) HOP-62 | 0 |
| Lung ca. (non-s.cl) NCI-H522 | 0 |
| Lung ca. (squam.) SW 900 | 0.85 |
| Lung ca. (squam.) NCI-H596 | 13.15 |
| Mammary gland | 7.04 |
| Breast ca.* (pl. effusion) MCF-7 | 100 |
| Breast ca.* (pl.ef) MDA-MB-231 | 0.02 |
| Breast ca.* (pl. effusion) T47D | 26.53 |
| Breast ca. BT-549 | 0.03 |
| Breast ca. MDA-N | 0.02 |
| Ovary | 3.51 |
| Ovarian ca. OVCAR-3 | 1.86 |
| Ovarian ca. OVCAR-4 | 0.10 |
| Ovarian ca. OVCAR-5 | 0 |
| Ovarian ca. OVCAR-8 | 0.50 |
| Ovarian ca IGROV-1 | 22.26 |
| Ovarian ca.* (ascites) SK-OV-3 | 11.13 |
| Uterus | 17.51 |
| Placenta | 1.27 |
| Prostate | 7.63 |
| Prostate ca.* (bone met)PC-3 | 0.03 |
| Testis | 1.13 |
| Melanoma Hs688(A).T | 0.02 |
| Melanoma* (met) Hs688(B).T | 0 |
| Melanoma UACC-62 | 0.03 |
| Melanoma M14 | 0 |
| Melanoma LOX IMVI | 0.02 |
| Melanoma* (met) SK-MEL-5 | 0.02 |
| Melanoma SK-MEL-28 | 0 |

### EXAMPLE 16

### Inhibition of Serine Protease Activity by the Protein Encoded by Clone 11692010.0.51, a PRO3 Nucleic Acid

Human Embryonic Kidney (HEK) 293 cells were grown in Dulbecco's modified eagle's medium (DMEM) with 10% fetal bovine serum medium to approximately 90% confluence. The cells were transfected with pCEP4/Sec (mock transfection vector) or pCEP4/Sec-11692010 (see, Example 6, supra) using Lipofectamnine 2000® (Gibco/BRL/Life Technologies, Rockville, Md.) according to the manufacturer's specifications. Transfected cells were incubated for 2 days with DMEM, and conditioned medium was then prepared by collection of cell supernatants.

The conditioned medium was enriched by TALON® metal affinity chromatography (Clontech; Palo Alto, Calif.) which is intended for the purification of 6×His protein fusions. In brief, the procedure was as follows. Seven ml of conditioned medium was incubated with 1 ml of TALON® metal affinity resin in spin columns. The spin columns were initially washed twice with 1 ml of Phosphate-buffered saline (PBS). The columns were then eluted twice with 0.65 ml of PBS/0.5M imidazole, pH 8.0 and the eluates were pooled. Imidazole was removed by buffer-exchange dialysis into PBS using Microcon® centrifugal filter devices (Millipore Corp.; Bedford, Mass.). The conditioned medium enriched in the 11692010 gene product was stored at 4° C.

In order to determine the ability of the 11692010 gene product to inhibit protease activity, the encoded protein was added in two different aliquot sizes (i.e., 25 μl and 50 μl) to a standard dilution of trypsin containing approximately 350 ng of enzyme. The resulting mixtures and appropriate positive and negative controls (i.e., serum and conditioned medium from mock transfection, respectively) were then assayed for trypsin activity using the PDQ Protease Assay™ (Athena Environmental Sciences, Inc.; Baltimore, Md.). In brief, this assay is a colorimetric assay using a proprietary substrate (i.e., a cross-linked matrix containing protein and a dye-protein conjugate) and is capable of identifying a wide range of proteases. Test samples containing protease activity and putative inhibitory substances were aliquoted into vials and incubated at 37° C. for 8 hours. Protease activity was detected spectrophotometrically at 450 nm with increasing optical density being proportional to increasing enzyme activity.

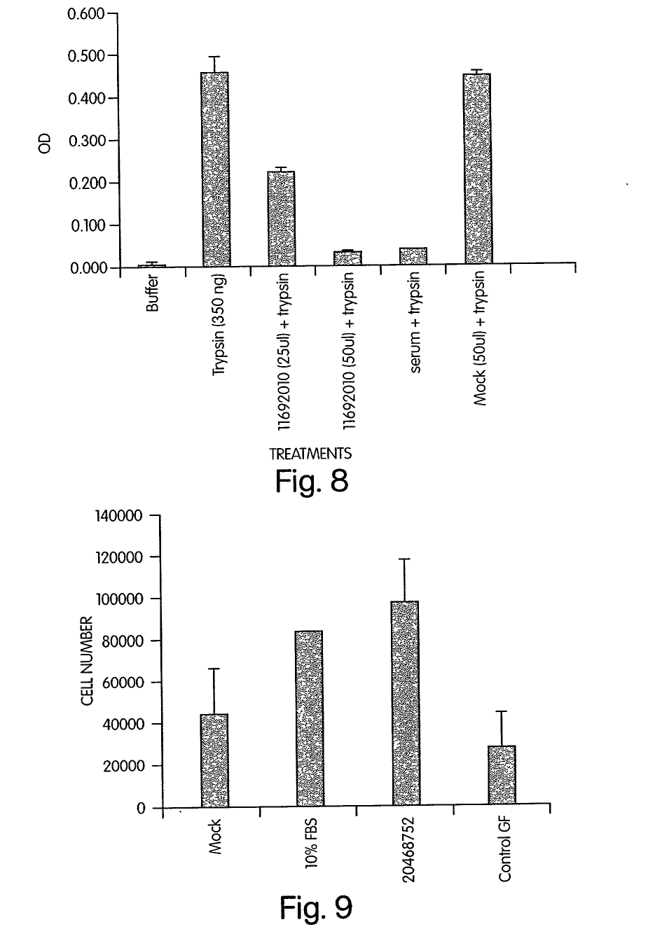

The results, shown in FIG. 8, indicate that the 11692010 gene product inhibits trypsin at a 50% inhibitory level corresponding to the addition of 25 μl of enriched, conditioned medium. It should be noted that this 50% level is relative to trypsin with no addition, or the addition of conditioned medium from the mock transfection.

Proteins exhibiting some similarity to the clone 11692010.0.51 protein are thought to be potentially useful for: (i) the stimulation of growth and motility of keratinocytes; (ii) the inhibition of the growth of cancer cells (e.g., melanomas); (iii) modulation of angiogenesis and tumor vascularisation; (iv) modulation of skin inflammation; and (v) modulation of epithelial cell growth.

Additionally, the protein encoded by Clone 11692010.0.51 also has some degree of similarity to fibromodulin, a protein that potentially regulates extracellular matrix remodeling. As disclosed herein, the protein encoded by Clone 11692010.0.51 has been shown here to inhibit protease activity, it is possible that this protein may also act to inhibit tumor cell metastasis and invasion.

### EXAMPLE 17

### Induction of Proliferation of NHost Cells by the Protein Encoded by Clone 20468752.0.18-U, a PRO2 Nucleic Acid

Human primary osteoblast cells (NHost; Clonetics; San Diego, Calif.) were plated at 40% confluency and cultured in DMEM supplemented with 10% fetal bovine serum or 10% calf serum for 24 hours. The culture medium was removed and replaced with an equivalent volume of enriched conditioned medium prepared as described in Example 16, with the exception that the transfection was performed using pCEP4/Sec-20468752 (see, Example 4, supra) or pCEP4/Sec (mock transfection vector; see, Example 3, supra). After approximately 48 hours, the cells were photographed with a Zeiss Axiovert 100. Cell numbers were then determined by trypsinization, followed by counting using a Coulter Z1 Particle Counter.

Treatment of the NHost cells with conditioned medium from 20468752.0.18-U-transfected HEK 293 cells resulted in a 2-fold increase in cell number over a two-day period (see, FIG. 9) as compared to mock transfection. Cells treated with a negative control containing an unrelated growth factor exhibited no growth (FIG. 9).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A substantially purified polypeptide comprising an amino acid sequence selected from any one of the following:
(a) a polypeptide of SEQ ID NO:6;
(b) a polypeptide having one or more conservative amino acid substitutions to the polypeptide of SEQ ID NO:6; or
(c) a mutant or variant of the polypeptide of SEQ ID NO:6.

2. A vector which encodes for the polypeptide of claim 1.

3. A cell comprising the vector of claim 2.

4. The cell of claim 3, wherein said cell is a prokaryotic or eukaryotic cell.

5. A process of producing a polypeptide of SEQ ID NO:6, the process comprising:
(a) providing the cell of claim 4;
(b) culturing said cell under conditions sufficient to express the SEQ ID NO:6 polypeptide; and
(c) recovering said SEQ ID NO:6 polypeptide,
thereby producing said SEQ ID NO:6 polypeptide.

6. A method of diagnosing a pathological condition associated with aberrant SEQ ID NO:6 polypeptide expression or activity in a subject, the method comprising:
(a) providing a protein sample from said subject;
(b) providing a control protein sample;
(c) measuring the amount of SEQ ID NO:6 polypeptide in said subject sample; and
(d) comparing the amount of SEQ ID NO:6 polypeptide in said subject protein sample to the amount of SEQ ID NO:6 polypeptide in said control protein sample,
wherein an alteration in the amount of SEQ ID NO:6 polypeptide in said subject protein sample relative to the amount of SEQ ID NO:6 polypeptide in said control protein sample indicates the subject has said pathological condition.

7. The method of claim 6, wherein said SEQ ID NO:6 polypeptide is detected using an antibody.

8. The method of claim 6, wherein said pathological condition is cancer.

9. A method for treating, preventing or delaying a pathological condition associated with aberrant SEQ ID NO:6 expression or activity in a subject, the method comprising administering to a subject in which said treatment, prevention or delay is desired the polypeptide of claim 1 in amount sufficient to treat, prevent or delay said pathological condition in said subject.

10. A method for identifying a compound that binds the polypeptide of claim 1, the method comprising:
(a) contacting SEQ ID NO:6 protein with a compound; and
(b) determining whether said compound binds SEQ ID NO:6 protein.

11. The method of claim 10, wherein binding of said compound to SEQ ID NO:6 is determined by a protein assay.

12. A compound identified by the method of claim 11.

13. A method for identifying a compound that modulates the activity of a SEQ ID NO:6 protein, the method comprising:
(a) contacting SEQ ID NO:6 protein with a compound; and
(b) determining whether SEQ ID NO:6 protein activity has been altered.

14. A compound identified by the method of claim 13.

15. A pharmaceutical composition comprising the polypeptide of claim 1 and a pharmaceutically-acceptable carrier.
